(19) United States
(12) Patent Application Publication
(10) Pub. No.: US 2014/0328842 A1
(43) Pub. Date: Nov. 6, 2014

(54) Predicting Responsiveness to Antibody Maintenance Therapy

(71) Applicant: PIKAMAB, Inc., Palo Alto, CA (US)

(72) Inventor: Vijay Ramakrishnan, Palo Alto, CA (US)

(21) Appl. No.: 14/364,856

(22) PCT Filed: Dec. 12, 2012
(86) PCT No.: PCT/US12/69323

§ 371 Date: Jun. 12, 2014

Related U.S. Application Data
(60) Provisional application No. 61/569,672, filed on Dec. 12, 2011.

(51) Int. Cl. C12Q 1/68

(52) U.S. Cl. CPC C12Q 1/6886; C12Q 2600/106
USPC 424/133.1; 424/174.1

(57) ABSTRACT

Methods, reagents and kits are provided for predicting whether an individual suffering from an antibody dependent cell-mediated cytotoxicity (ADCC)-treatable disease is responsive to an antibody maintenance therapy. The methods, reagents, and kits find a number of uses, including, for selecting individuals who will be responsive for treatment with an antibody maintenance therapy, for determining the appropriate maintenance therapy for an individual suffering from an ADCC-treatable disease, for determining the optimal regimen for antibody maintenance therapy, and for treating an individual with an antibody maintenance therapy based on stratification into responsive groups.

### CROSS-REFERENCES TO RELATED APPLICATIONS

This application claims benefit of U.S. Provisional Patent Application No. 61/569,672 filed Dec. 12, 2011, the entire contents of which is incorporated herein by this reference.

### BACKGROUND

Antibody dependent cell-mediated cytotoxicity (ADCC) has become a powerful tool for halting and in some instances reversing the progression of a wide variety of diseases and disorders, including, for example, neoplastic diseases, autoimmune diseases, microbial infections, and transplant rejections. In some instances, ADCC is being harnessed for maintenance therapy, i.e. therapy to stabilize the improvement in health obtained with induction therapy, or to “consolidate” the gains obtained with induction therapy. An effective and practical diagnostic protocol which could provide information as to whether a patient will or will not be responsive to an antibody-based maintenance therapy would be desirable for a number of reasons, including avoidance of delays in alternative treatments, elimination of exposure to adverse effects of the antibodies, and reduction of unnecessary treatment expenses. The methods, reagents, and kits presented herein address some of these issues.

### SUMMARY

Methods of treatment are provided using antibody maintenance therapy, particularly providing improved responsiveness to treatment. Methods, reagents and kits are provided for predicting whether an individual suffering from an antibody dependent cell-mediated cytotoxicity (ADCC)-treatable disease is responsive to an antibody maintenance therapy. The methods, reagents, and kits find a number of uses, e.g., for selecting individuals for treatment with an antibody maintenance therapy, for determining the appropriate maintenance therapy for an individual suffering from an ADCC-treatable disease, for determining the optimal regimen for antibody maintenance therapy, etc.

The present invention is based, in part, upon the observation that polymorphisms in human Fc receptors, FcγRIIIA and FcγRIIA, independently and together dictate therapeutic response rates of ADCC-treatable diseases to antibody maintenance therapy. Thus, stratifying patients into variant genotype groups, e.g., into one of the nine genotype groups corresponding to the major polymorphisms at the FcγRIIIA V/F¹⁵⁸ and FcγRIIA H/R¹³¹ locations can be clinically relevant and a significant factor in determining responsiveness to antibody maintenance treatment.

In some aspects of the invention, methods are provided for predicting responsiveness of an individual having an antibody dependent cell-mediated cytotoxicity (ADCC)-treatable disease or disorder to an antibody maintenance therapy, e.g., a monoclonal antibody maintenance therapy comprising an antibody such as RITUXAN®, MABTHERA®, ARZERRA®, CAMPATH®, ZENAPAX®, HERCEPTIN®, PERJETA®, XOLAIR®, RAPTIVA®, AVASTIN®, REMICADE®, HUMIRA®, ERBITUX®, SIMULECT®, SYNAGIS®, VECTIBIX®, TYSABRI®, MYLOTARG®, REOPRO®, LUCENTIS®, SOLIRIS®, BENLYSTA®, BEXXAR®, ZEVALIN®, ADCETRIS®, ILARIS®, CIMZIA®, PROLIA®, XGEVA®, LYMPHOCIDE®, PANOREX®, SIMPONI®, YERVOY®, ORTHOCLONE OKT3®, ACTEMRA®, ROACTEMRA®, etc. (see Table 3), and/or for selecting a patient having an ADCC-treatable disease or disorder for treatment with an antibody maintenance therapy, e.g., a monoclonal antibody maintenance therapy comprising an antibody such as RITUXAN®, MABTHERA®, ARZERRA®, CAMPATH®, ZENAPAX®, HERCEPTIN®, PERJETA®, XOLAIR®, RAPTIVA®, AVASTIN®, REMICADE®, HUMIRA®, ERBITUX®, SIMULECT®, SYNAGIS®, VECTIBIX®, TYSABRI®, MYLOTARG®, REOPRO®, LUCENTIS®, SOLIRIS®, BENLYSTA®, BEXXAR®, ZEVALIN®, etc.

In some embodiments, the methods comprise genotyping the individual for one or more FcγR polymorphisms to obtain a result; and predicting the responsiveness of the individual to an antibody maintenance therapy or selecting the individual to be treated with the antibody maintenance therapy based upon the result. In some embodiments, the one or more FcγR polymorphisms are selected from an FcγRIIA polymorphism and an FcγRIIIA polymorphism. In some embodiments, both an FcγRIIA polymorphism and an FcγRIIIA polymorphism are assessed. In some embodiments, the FcγRIIA polymorphism is at amino acid residue 131. In some embodiments, the FcγRIIIA polymorphism is at amino acid residue 158. In some embodiments, a H/H¹³¹ FcγRIIA, V/V¹⁵⁸ FcγRIIIA genotype is predictive of excellent responsiveness to the antibody maintenance therapy; a H/H¹³¹ FcγRIIA, V/F¹⁵⁸ FcγRIIIA genotype; a H/H¹³¹ FcγRIIA, F/F¹⁵⁸ FcγRIIIA genotype; a H/R¹³¹ FcγRIIA, V/V¹⁵⁸ genotype; or a R/R¹³¹ FcγRIIA, V/V¹⁵⁸ FcγRIIIA genotype is predictive of good responsiveness to the antibody maintenance therapy; a H/R¹³¹ FcγRIIA, V/F¹⁵⁸ FcγRIIIA genotype is predictive of moderate responsiveness to the antibody maintenance therapy; a H/R¹³¹ FcγRIIA, F/F¹⁵⁸ FcγRIIIA genotype; or an R/R¹³¹ FcγRIIA, V/F¹⁵⁸ FcγRIIIA genotype is predictive of weak responsiveness to the antibody maintenance therapy; and a R/R¹³¹ FcγRIIA, F/F¹⁵⁸ FcγRIIIA genotype is predictive of poor responsiveness to the antibody maintenance therapy.

In some embodiments, the individual exhibits no symptoms of the ADCC-treatable disease or disorder. In other embodiments, the individual exhibits symptoms of the ADCC-treatable disease or disorder. In some embodiments, the individual previously received antibody maintenance therapy for the disease or disorder. In some embodiments, the individual is receiving antibody maintenance therapy for the disease or disorder. In some embodiments, the ADCC-treatable disease or disorder is selected from the group consisting of a neoplastic disease, e.g., B non-Hodgkin's lymphoma (B-NHL), e.g., follicular lymphoma (FL); an autoimmune disease; an inflammatory disease; a microbial infection; and an allograft rejection. In some embodiments, the antibody maintenance therapy comprises an anti-CD20 antibody. In certain embodiments, the anti-CD20 antibody is rituximab. In some embodiments, the maintenance therapy is administered regularly. In other embodiments, the maintenance therapy is administered as needed.

In some embodiments, depletion of a cell population in a subject is a function of ADCC, and thus can be used as another measure to predict responsiveness to antibody maintenance therapy. Accordingly, in some embodiments, a method for predicting responsiveness to antibody maintenance therapy can comprise measuring depletion of a cell population targeted by an antibody induction therapy or an antibody maintenance therapy to obtain a result; and predicting the responsiveness of the individual to an antibody maintenance therapy or selecting the individual to be treated with the antibody maintenance therapy based upon the result. In some embodiments, the individual exhibits no symptoms of the ADCC-treatable disease or disorder. In other embodiments, the individual exhibits symptoms of the ADCC-treatable disease or disorder. In some embodiments, the individual previously received antibody maintenance therapy for the disease or disorder. In some embodiments, the individual is receiving antibody maintenance therapy for the disease or disorder. In some embodiments, the ADCC-treatable disease or disorder is a neoplastic disease, an autoimmune disease, a microbial infection, or allograft rejection. In some embodiments, the targeted cell population is B cells. In some embodiments, the neoplastic disease is B non-Hodgkin's lymphoma (B-NHL), e.g., follicular lymphoma (FL). In some embodiments, the antibody induction therapy is induction therapy comprising an anti-CD20 antibody, e.g., rituximab. In some embodiments, the antibody maintenance therapy is administered regularly. In other embodiments, the antibody maintenance therapy is administered as needed.

In some embodiments, repopulation of a cell population in a subject is a function of ADCC, and thus can be used to predict responsiveness to antibody maintenance therapy. Accordingly, in some embodiments, a method of predicting responsiveness to antibody maintenance therapy can comprise evaluating repopulation of the target cell population in the individual after depletion with the antibody of interest. In some embodiments, the method further comprises genotyping the individual for an FcγRIIA polymorphism and an FcγRIIIA polymorphism to get a genotyping result, wherein predicting responsiveness of the individual to an antibody maintenance therapy is based upon the result of the depletion measurement, the repopulation measurement and the genotyping result.

In some aspects of the invention, methods are provided for treating an individual having an ADCC-treatable disease or disorder with an antibody maintenance therapy, e.g., a monoclonal antibody maintenance therapy comprising an antibody such as RITUXAN®, MABTHERA®, ARZERRA®, CAMPATH®, ZENAPAX®, HERCEPTIN®, PERJETA®, XOLAIR®, RAPTIVA®, AVASTIN®, REMICADE®, HUMIRA®, ERBITUX®, SIMULECT®, SYNAGIS®, VECTIBIX®, TYSABRI®, MYLOTARG®, REOPRO®, LUCENTIS®, SOLIRIS®, BENLYSTA®, BEXXAR®, ZEVALIN®, ADCETRIS®, ILARIS®, CIMZIA®, PROLIA®, XGEVA®, LYMPHOCIDE®, PANOREX®, SIMPONI®, YERVOY®, ORTHOCLONE OKT3®, ACTEMRA®, ROACTEMRA®, etc.

In some embodiments, the methods comprise genotyping the individual for one or more FcγR polymorphisms, classifying the individual into a responsiveness group based on the genotyping; and administering antibody maintenance therapy to the individual either as a regular regimen or an as needed regimen, wherein the regimen is selected based on the individual's classification in a responsiveness group. In some embodiments, the one or more FcγR polymorphisms is selected from an FcγRIIA polymorphism, e.g., at amino acid residue 131, and an FcγRIIIA polymorphism, e.g., at amino acid residue 158. In some embodiments, the method further comprises administering induction therapy to the individual. In some embodiments, the induction therapy comprises chemotherapy. In some embodiments, the induction therapy comprises an antibody. In some embodiments, the antibody maintenance therapy is administered regularly. In other embodiments, the antibody maintenance therapy is administered as needed. In some such embodiments, the method further comprises evaluating the repopulation of the target cell population in the individual after depletion with the antibody of interest.

In some embodiments, depletion of a cell population in a subject is a function of ADCC, and thus can be used to treat a subject having an ADCC treatable disease. Accordingly, in some embodiments, a method for selecting an treatment regimen or method can comprise measuring depletion of a cell population targeted by an antibody induction therapy or an antibody maintenance therapy to obtain a result; classifying the individual into a responsiveness groups based on the genotyping; and administering antibody maintenance therapy to the individual either as a regular regimen or an as needed regimen, wherein the regimen is selected based on the individual's classification in a responsiveness group. In some embodiments, the antibody maintenance therapy is administered regularly. In other embodiments, repopulation of a cell population in a subject is a function of ADCC, and thus the antibody maintenance therapy is administered as needed based on the repopulation. In some such embodiments, the method further comprises evaluating the repopulation of the target cell population in the individual after depletion with the antibody of interest.

In some aspects of the invention, kits are provided for predicting responsiveness of an individual having an ADCC-treatable disease or disorder to an antibody maintenance therapy. In some embodiments, the kit comprises one or more of: (a) an element for genotyping the patient to identify an FcγRIIA polymorphism; and (b) an element for genotyping the patient to identify an FcγRIIIA polymorphism. In some embodiments, the FcγRIIA polymorphism is a polymorphism at amino acid residue 131. In some embodiments, the FcγRIIIA polymorphism is a polymorphism at amino acid residue 158. In some embodiments, the kit further comprises a reference that correlates a genotype in the patient with a patient group having known responsiveness to the antibody maintenance therapy. In some embodiments, the ADCC-treatable disease or disorder is a neoplastic disease, e.g., NHL, e.g., FL; an autoimmune disease; a microbial infection; or allograft rejection.

In some embodiments, the kit comprises an element for measuring the number of cells in a sample from an individual having an ADCC-treatable disease or disorder, wherein the cells are cells targeted for depletion by an antibody induction therapy or an antibody maintenance therapy. In some embodiments, the element is an antibody, e.g., an antibody that is specific for B cells. In some embodiments, the kit further comprises a reference that correlates the extent of depletion of targeted cells following antibody induction therapy or antibody maintenance therapy with responsiveness to an antibody maintenance therapy. In some embodiments, the kit further comprises a reference that correlates the extent of repopulation of targeted cells following antibody induction therapy or antibody maintenance therapy with responsiveness to an antibody maintenance therapy (e.g., reference index or reference stratification). In some embodiments, the ADCC-treatable disease or disorder is a neoplastic disease, e.g., NHL, e.g., FL; an autoimmune disease; an inflammatory disease; a microbial infection; or allograft rejection.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1 provides a diagram of the 2-dimensional stratification of patients into a 3×3 Matrix based on FcγRIIIA V/F¹⁵⁸ and FcγRIIA H/R¹³¹ polymorphisms.

FIG. 2 provides a diagram of the interrelatedness and interdependence of ADCC, activity measures of ADCC functions, target cell variation (which may be depletion or expansion, depending upon the cell subset), and clinical/therapeutic response rates with FcγRIIA and FcγRIIIA polymorphisms when the major mechanism of action of an antibody therapy is ADCC. ADCC functional assays or evaluation of ADCC cell effector subsets or target cell variation itself can be an independent or supplemental predictor of clinical/therapeutic response rates.

### DETAILED DESCRIPTION

The present disclosure provides methods, reagents and kits for predicting whether an individual or subject suffering from an antibody dependent cell-mediated cytotoxicity (ADCC)-treatable disease is responsive to an antibody maintenance therapy. The methods, reagents, and kits find a number of uses, including, among others, selecting an individual or subject for treatment with an antibody maintenance therapy; determining the appropriate treatment option, including maintenance therapy, for an individual suffering from an ADCC-treatable disease; determining an optimal regimen for antibody maintenance therapy; and treating an individual or a subject suffering from an ADCC treatable disease with antibody maintenance therapy.

Before the present invention is further described, it is to be understood that this invention is not limited to particular embodiments described, as such may, of course, vary. It is also to be understood that the terminology used herein is for the purpose of describing particular embodiments only, and is not intended to be limiting.

It is also to be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In addition, the use of “or” means “and/or” unless stated otherwise. Similarly, “comprise,” “comprises,” “comprising,” “include,” “includes,” and “including” are interchangeable and not intended to be limiting. Where descriptions of various embodiments use the term “comprising,” those skilled in the art would understand that in some specific instances, an embodiment can be alternatively described using language “consisting essentially of” or “consisting of.”

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Although any methods and materials similar or equivalent to those described herein can also be used in the practice or testing of the present invention, the preferred methods and materials are now described. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In some embodiments, methods recited herein may be carried out in any order of the recited events which is logically possible, as well as the recited order of events.

The section headings used herein are for organizational purposes only and not to be construed as limiting the subject matter described.

### DEFINITIONS

Unless defined otherwise, all technical and scientific terms used herein have the same meaning as commonly understood by one of ordinary skill in the art to which this invention belongs. Accordingly, the following terms are intended to have the following meanings:

“Subject,” “individual, “host,” or “patient” generally refers to mammals, including humans and other non-human mammals, including, but not limited to, cats, dogs, rodents, rats, mice, hamsters, rabbits, horses, cows, sheep, pigs, goats, etc. As used herein, “subject,” “individual, “host” or “patient” includes one who is to be tested, or has been tested for prediction, assessment or diagnosis of an ADCC disease or disorder.

“Antibody dependent cell-mediated cytotoxicity” and “ADCC” refer to a cell-mediated reaction in which cytotoxic cells, typically cytotoxic cells that express Fc receptors (FcRs), recognize bound antibody on a target cell and subsequently cause destruction or killing of the target cell, e.g., by phagocytosis; antibody-dependent ADCC-mediated cell killing/lysis; ADCC-mediated apoptosis; and trogocytosis (antibody-dependent cytotoxicity mediated by polymorphonuclear granulocytes). Known cytotoxic cells mediating ADCC include, among others, Natural Killer (NK) cells, neutrophils, eosinophils, monocytes and macrophages. The primary cells for mediating ADCC, NK cells, express FcγRIII, whereas monocytes express FcγRI, FcγRII and FcγRIII.

“ADCC effector cells” refers to cytotoxic cells, e.g., leukocytes, which express one or more FcRs and perform effector functions. Examples of human leukocytes which mediate ADCC include peripheral blood mononuclear cells (PBMC), natural killer (NK) cells, monocytes, macrophages, cytotoxic T cells, eosinophils and neutrophils. Effector cells may be isolated from a native source thereof, e.g., from blood or PBMCs by methods known in the art.

“Isolated cells” refer to a preparation of cells that have been separated from other components in a mixture containing the cells. In some embodiments, the cells are in the form of a “substantially purified” cell preparation.

“Responsiveness” in reference to a subject refers to a treatment outcome or a clinical outcome of a treatment or therapy for a disease or disorder. The treatment outcome or clinical outcome can be measured according to standards recognized in the art for a specific disease or disorder.

“ADCC treatable disease or disorder” refers to a disease or disorder that can be treated by promoting antibody dependent cell-mediated cytotoxicity (ADCC). For example, an ADCC treatable disease or disorder can be treated with an antibody whose therapeutic mechanism comprises whole or in part ADCC by binding to a target cell and promoting ADCC.

“ADCC activity” refers to an ADCC reaction. ADCC activity can be assessed using an in vitro assay, e.g., a ⁵¹Cr release assay, using peripheral blood mononuclear cells (PBMC) and/or NK effector cells as described in Shields et al. (2001) J. Biol. Chem., 276:6591-6604, U.S. Pat. No. 5,500,362 or 5,821,337, or another suitable method. Alternatively, or additionally, ADCC activity of the molecule of interest may be assessed in vivo, e.g., in an animal model such as that disclosed in Clynes et al., (1998) Proc Natl Acad Sci. USA 95:652-656.

“Antibody” refers to an immunoglobulin or fragment thereof, and encompasses any such polypeptide comprising an antigen-binding fragment of an antibody. The term includes but is not limited to polyclonal, monoclonal, monospecific, multispecific (e.g., bispecific antibodies), humanized, human, single-chain, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments and in vitro generated antibodies so long as they exhibit the desired biological activity. Antibody for the purposes herein will generally have an Fc portion and be capable of mediating or participating in ADCC reaction by binding to an Fc receptor.

“Chimeric antibody” refers to an antibody made up of components from at least two different sources. A chimeric antibody can comprise a portion of an antibody derived from a first species fused to another molecule, e.g., a portion of an antibody derived from a second species. In some embodiments, a chimeric antibody comprises a portion of an antibody derived from a non-human animal, e.g., mouse or rat, fused to a portion of an antibody derived from a human. In some embodiments, a chimeric antibody comprises all or a portion of a variable region of an antibody derived from a non-human animal fused to a constant region of an antibody derived from a human.

“Humanized antibody” refers to an antibody that comprises a donor antibody binding specificity, i.e., the CDR regions of a donor antibody, typically a mouse monoclonal antibody, grafted onto human framework sequences. A “humanized antibody” as used herein typically binds to the same epitope as the donor antibody.

“Fully human antibody” refers to an antibody that comprises human immunoglobulin protein sequences only. A fully human antibody may contain murine carbohydrate chains if produced in a mouse, in a mouse cell, or in a hybridoma derived from a mouse cell.

“Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (Fc fragment of IgG, low affinity IIA, receptor), also known as FcγR2A, Fcγ2A and CD32, the nucleotide and amino acid sequences for which may be found at Genbank Accession Nos. NM_{—}021642 or M28697. FcγRIII receptors include FcγRIIIA (“Fc fragment of IgG, low affinity IIIA, receptor”), also known as FcγR3A and CD16a, the nucleotide and amino acid sequences for which may be found at Genbank Accession Nos. NM_{—}000569, NM_{—}001127592.1, NM_{—}001127593.1, NM_{—}001127595.1, and NM_{—}001127596.1). FcRs are reviewed in Ravetch and Kinet, (1991) Annu. Rev. Immunol. 9:457-92; Capel et al., (1994) Immunomethods 4:25-34; and de Haas et al., (1995) J. Lab. Clin. Med. 126:330-41.

“Genotype” refers to the alleles present in DNA from a subject or patient, where an allele can be defined by the particular nucleotide(s) present in a nucleic acid sequence at a particular site(s). Often a genotype is the nucleotide(s) present at a single polymorphic site known to vary in the population. In some embodiments, a “genotype” is reflected in an expressed protein, which may be detected by known procedures, such as by using antibodies or protein sequencing.

“Polymorphism” refers to the coexistence of more than one form of a gene or portion thereof. While a polymorphism is present at the nucleotide level, it may also manifest in an expressed gene product, e.g., a protein.

“Allele,” which is used interchangeably herein with “allelic variant” and “variant allele”, refers to alternative forms of a gene or portions thereof. Alleles occupy the same locus or position on homologous chromosomes. When a subject has two identical alleles of a gene, the patient is said to be homozygous for the gene or allele. When a subject has two different alleles of a gene, the patient is said to be heterozygous for the gene. Alleles of a specific gene, including FcγIIA, can differ from each other in a single nucleotide, or several nucleotides, and can include substitutions, deletions, and insertions of nucleotides. An allele of a gene can also be a form of a gene containing one or more mutations.

“Fcγ receptor polymorphism” refers to more than one form of a gene for a specific Fcγ receptor. By an FcγRIIA polymorphism, it is meant a polymorphism in the FcγRIIA gene which results in an amino acid substitution in the FcγRIIA protein. By an FcγRIIIA polymorphism, it is meant a polymorphism in the FcγRIIIA gene which results in an amino acid substitution in the FcγRIIIA protein.

“Amino acid residue” and “amino acid position” are used interchangeably herein to refer to the position of the specified amino acid in the polypeptide chain. In some embodiments, the amino acid residue can be represented as “X^{N}”, where X represents the amino acid and the N represents its position in the polypeptide chain. Where two or more variations, e.g., polymorphisms, occur at the same amino acid position, the variations can be represented with a “/” separating the polymorphisms. For example two possible polymorphisms can be represented as “X/Y^{N}”, where X and Y represent the possible amino acids and N represents the position in the polypeptide chain. In some embodiments, the two possible variations can also be represented as “XNY”, where X, N and Y are as described above, e.g., H131R.

“Maintenance therapy” refers to a therapy, therapeutic regimen or course of therapy which is administered subsequent to an induction therapy (an initial course of therapy administered to an individual or subject with a disease or disorder). In some embodiments, therapy that includes maintenance therapy is considered maintenance therapy. Maintenance therapy can be used to halt or reverse the progression of the disease/disorder), to maintain the improvement in health achieved by induction therapy and/or enhance, or “consolidate”, the gains obtained by induction therapy.

“Antibody maintenance therapy” refers to an antibody therapy, i.e., a therapy comprising one or more antibodies, which is administered as maintenance therapy in the therapeutic regimen or course of therapy.

“Induction therapy” refers to the first course of treatment in treating a disease, disorder or medical condition.

“Responsiveness” in reference to a subject refers to a treatment outcome or a clinical outcome of a treatment or therapy for a disease or disorder. The treatment outcome or clinical outcome can be measured according to standards recognized in the art for a specific disease or disorder.

“Predicting” refers to determining the probability or likelihood of a particular outcome or event. In reference to responsiveness to treatment, the term refers to the likelihood of a particular treatment outcome or clinical outcome.

“Predicting responsiveness”, or “providing a prognosis” or “prognosing”, it is meant predicting whether or not the antibody maintenance therapy will have an impact on disease progression.

“Overall survival” or “OS” refers to the time (in years) measured from diagnosis, study entry, or early randomization (depending on the study design) to death from any cause. Overall survival is a term that denotes the chances of staying alive for a group of individuals suffering from a disease or disorder.

“Progression free survival” or “PFS” refers to the time (in years) measured from the start of maintenance therapy during which the disease being treated does not worsen. Progression free survival is a metric that denotes the chances of a disease stabilizing or being reversed in a group of individuals suffering from the disease. For instance, it denotes the percentage of individuals in the group who are likely to be as healthy if not healthier after a particular period of time following the start of maintenance therapy.

“Relapse-free survival” or “RFS” refers to the time (in years) measured from diagnosis to first recurrence of the disease, e.g., first recurrence of a malignancy in a neoplastic disease. RFS is defined only for patients achieving complete remission, and is measured from the date of achievement of a remission until the date of relapse or death from any cause.

“Event-free survival” or “EFS” refers to the time (in years) measured from diagnosis to the first subsequent event associated with the disease, e.g., complications from the disease, first malignancy recurrence, or death. EFS is defined for all patients of a trial, and is measured from the date of entry into a study to the date of induction treatment failure, or relapse from complete remission (CR) or CRi, or death from any cause.

“Antibody therapy” refers to a medical treatment involving an antibody. An “antibody therapy” in reference to an ADCC treatable disease refers to an antibody that has a therapeutic mechanism based wholly or in part on ADCC.

“Time to Progression” or “TTP” refers to a measure of time after a disease is diagnosed (or treated) until the disease begins to worsen.

“Chemotherapy” or “chemotherapeutic regimen” refers to the administration of at least one chemotherapy agent that is used to treat a disease or disorder. Chemotherapy agents may be administered to a subject in a single bolus dose, or may be administered in smaller doses over time. A single chemotherapeutic agent may be used (single-agent therapy) or more than one agent may be used in combination as combination therapy. A chemotherapeutic agent as used herein comprises a non-biologic therapeutic, including small molecule drugs, peptide drugs, anti-sense nucleic acids, etc.

“Administering an antibody therapy” or “administering an antibody maintenance therapy” refers to administering an antibody to a subject for purposes of therapy (e.g., induction therapy) or maintenance therapy, respectively.

“Administered regularly” refers to administration of a therapeutic (e.g., drug or biologic) or treatment at periodic intervals.

“Administered as needed” refers to administration of a therapeutic (e.g., drug or biologic) or treatment when the subject suffers a relapse or diagnostics indicate the need for retreatment (e.g., target cell repopulation), and is generally determined by a medical doctor of skill in the art.

“Course of treatment” or “course of therapy” refers to administration of a drug or therapeutic for a period of time as part of a defined treatment plan. The course of treatment or therapy can be a first course, second course, third course, etc. The courses may or may not use the same therapeutic. The drug or therapeutic can be administered as a single dose or in multiple doses in a single course. Multiple doses in a course of therapy can be administered over a period of time, such as days, weeks or months, depending on the therapeutic and the disease or disorder to be treated.

“Stratifying” or “stratification” refers to classifying subjects into distinct groups based common characteristic(s) or trait(s). Stratification can be based on a single trait or two or more traits. When the occurrences of two or more characteristics or traits are statistically linked, one of the traits can be stratified based on the other trait. For example, when the genotype and responsiveness to treatment or therapeutic regimen are linked, responsiveness can be stratified or classified based on the genotype.

“Reference stratification” as used herein refers to an established stratification scheme that has stratified a treatment response/clinical outcome-genotype association, with statistically significant differences between the different groups in the stratification. Accordingly, a subject afflicted with an ADCC treatable disease whose genotype for the Fcγ receptor polymorphism (e.g., FcγIIA and/or FcγIIIA), is known can be compared to the reference stratification to identify the likelihood of the subject having a particular treatment outcome or clinical outcome, i.e., responsiveness, for an antibody maintenance therapy.

“Correlating,” “correlation,” “correlates,” as used herein refer to the establishment of a relationship, e.g., mutual or reciprocal, between genotype status and therapeutic efficacy of certain treatments as described herein. That is, correlating refers to relating the genotype status to responsiveness to treatment or therapy.

“Excluding a treatment or therapy” refers to removing a possible treatment from consideration, e.g., for use on a particular patient, based on the presence or absence of a particular variance(s) in one or more genes of that patient.

“Excluding a subject” refers to removing the subject from consideration of a treatment or therapy, including in reference to treatment or therapy in clinical trials, based on the presence or absence of a particular variance(s) in one or more genes of that patient.

“Selecting a treatment or therapy” refers to including a possible treatment for consideration, e.g., for treating a particular patient based on the presence or absence of a particular variance(s) in one or more genes of that patient.

“Selecting a subject” refers to including the subject for consideration of a treatment or therapy, including in reference to treatment or therapy in clinical trials, based on the presence or absence of a particular variance(s) in one or more genes of that patient.

“Neoplastic disease or disorder” refers to a disease state in a subject in which there are cells and/or tissues which proliferate abnormally. Neoplastic disorders can include, but are not limited to, cancers, sarcomas, tumors, leukemias, lymphomas, and the like. Hyperproliferative disorders, or malignancies, are conditions in which there is unregulated cell growth. The terms “cancer,” “neoplasm,” “hyperproliferative cell,” and “tumor” are used interchangeably herein to refer to cells which exhibit relatively autonomous growth, so that they exhibit an aberrant growth phenotype characterized by a significant loss of control of cell proliferation. Cancerous cells can be benign or malignant. Viral infections (e.g., HCV infection in B-cells) can lead to hyper(lympho)proliferative disorders.

“Autoimmune disease or disorder” refers to a disease state caused by immune-responsiveness against self-tissues and/or substances normally present in the body. It is generally associated with production of inflammatory factors, which further promote tissue destruction. Inflammatory macrophages, inflammatory NKT cells etc. can cause chronic inflammatory diseases such as atherosclerosis, Type-2 diabetes, sickle cell disease, and the like. Autoimmune diseases can be systemic or organ-specific. Examples of systemic autoimmune diseases include: multiple sclerosis, rheumatoid arthritis, systemic lupus erythematosis, ankylosing spondylitis, scleroderma and Sjogren's syndrome. Examples or organ-specific autoimmune diseases include: Addison's disease, Autoimmune hemolytic anemia, Goodpasture's syndrome, Grave's disease, Hashimoto's thyroiditis, idiopathic thrombocytopenia purpura, insulin-dependent diabetes mellitus, myasthenia gravis, pernicious anemia, poststreptococcal glomerulonephritis and psoriasis.

“Inflammatory disease or disorder” refers to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. In some embodiments, an inflammatory disease or disorder can be an aspect of other diseases, such as autoimmune diseases.

“Allograft rejection” refers to a reaction within a transplanted organ or tissue involving both immunologic and non-immunologic responses that ultimately lead to damage or necrosis of some or all of the transplanted organ or tissue. An “organ” refers to a part of the body of a subject exercising a specific function (such as a heart, kidney, liver, or lung). A “tissue” refers to a collection of similar cell types (such as epithelium, connective, muscle and nerve tissue). A “transplanted tissue or organ” is meant to refer to a tissue or organ taken from one subject and implanted into a subject other than the subject from which the organ or tissue was taken.

“Suffering from a disease or condition” means that a subject is either presently subject to the signs and symptoms, or is more likely to develop such signs and symptoms than a normal subject in the population. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

“Treatment” refers to a process that is intended to produce a beneficial change in the condition of a mammal, e.g., a human, often referred to as a patient. A beneficial change can, for example, include one or more of restoration of function, reduction of symptoms, limitation or retardation of progression of a disease, disorder, or condition or prevention, limitation or retardation of deterioration of a patient's condition, disease or disorder. In the context of ADCC-based therapy, “treatment” or “treatable” is meant the ADCC-based therapy achieves a desired pharmacologic and/or physiologic effect on the disease or disorder. The effect may be prophylactic in terms of completely or partially preventing the disease/disorder or symptom thereof and/or may be therapeutic in terms of a partial or complete cure for the disease/disorder and/or adverse effect attributable to the disease/disorder. The terms includes: (a) preventing the disease from occurring in a subject which may be predisposed to the disease but has not yet been diagnosed as having it; (b) inhibiting the disease, i.e., arresting its development; or (c) relieving the disease, i.e., causing remission or regression of the disease. The therapeutic agent may be administered before, during or after the onset of the disease or disorder. The treatment of ongoing disease, where the treatment stabilizes or reduces the undesirable clinical symptoms of the patient, is of particular interest. Such treatment is desirably performed prior to complete loss of function in the affected tissues.

“Anti-CD19 antibody” refers to an antibody that specifically recognizes cell surface protein CD19. As used herein “CD19” includes, for example, variants, isoforms, and species homologs of human CD19. Anti-CD19 antibodies may, in certain instance, cross-react with CD19 of more than one species. In some embodiments, the antibodies may be completely specific for one or more human CD19 proteins and may not exhibit species or other types of non-human cross-reactivity. The amino acid sequence of an exemplary human CD19 has Genbank/NCBI accession number NP_{—}001171569.1. Exemplary anti-CD19 antibodies are described in U.S. Pat. Nos. 7,109,304 and 8,147,831.

“Anti-CD20 antibody” refers to an antibody that specifically binds to cell surface protein CD20, which is expressed on certain cells of the immune system, specifically the B-lymphocyte differentiation antigen Bp35. As used herein “CD20” includes, for example, variants, isoforms, and species homologs of human CD20. Anti-CD20 antibodies may, in certain instances, cross-react with CD20 of more than one species. In some embodiments, the antibodies may be completely specific for one or more human CD20 proteins and may not exhibit species or other types of non-human cross-reactivity. The amino acid sequence of an exemplary human CD20 has Genbank/NCBI accession number NP_{—}068769.2. Exemplary anti-CD20 antibodies include rituximab, veltuzumab and others as described herein. Anti-CD20 antibodies are also described in U.S. Pat. Nos. 6,399,061 and 7,422,739.

“Anti-CD22 antibody” refers to refers to an antibody that specifically binds to cell surface protein CD22, which is expressed on B lymphocytes and is also known as “BL-CAM” and “LyB”. As used herein “CD22” includes, for example, variants, isoforms, and species homologs of human CD22. Anti-CD20 antibodies may, in certain instances, cross-react with CD22 of more than one species. In some embodiments, the antibodies may be completely specific for one or more human CD22 proteins and may not exhibit species or other types of non-human cross-reactivity. The amino acid sequence of an exemplary human CD22 has Genbank/NCBI accession number NP_{—}001172028.1. Exemplary anti-CD22 antibodies are described in U.S. Pat. Nos. 6,306,393 and 7,456,260.

“Anti-CD25 antibody” refers to an antibody that specifically binds to CD25, the alpha subunit of interleukin-2 receptor, also known as “p55” and “Tac (T cell activation) antigen”. As used herein “CD25” includes, for example, variants, isoforms, and species homologs of human CD25. Anti-CD25 antibodies may, in certain instances, cross-react with CD25 of more than one species. In some embodiments, the antibodies may be completely specific for one or more human CD25 proteins and may not exhibit species or other types of non-human cross-reactivity. The amino acid sequence of an exemplary human CD25 has Genbank/NCBI accession number NP_{—}000408.1. Exemplary anti-CD25 antibodies include daclizumab and basiliximab, and are also described in US patent publications 20090081219 and 20120244069.

“Anti-CD30 antibody” refers to an antibody that specifically binds to CD30, a lymphocyte activation marker that is a member of the tumor necrosis factor receptor family, and is also known as Ki-1 and TNFRSF8. As used herein “CD30” includes, for example, variants, isoforms, and species homologs of human CD30. Anti-CD30 antibodies may, in certain instances, cross-react with CD30 of more than one species. In some embodiments, the antibodies may be completely specific for one or more human CD30 proteins and may not exhibit species or other types of non-human cross-reactivity. The amino acid sequence of an exemplary human CD30 has Genbank/NCBI accession number NP_{—}001234.2. Exemplary anti-CD30 antibodies are described in U.S. Pat. Nos. 7,090,843, 7,387,776 and 7973136

“Anti-CD33 antibody” refers to refers to an antibody that specifically binds to CD33, a myeloid differentiation antigen and a member of the sialoadhesin family. It is also known as gp67 and SIGLEC-3. As used herein “CD33” includes, for example, variants, isoforms, and species homologs of human CD33. Anti-CD33 antibodies may, in certain instances, cross-react with CD33 of more than one species. In some embodiments, the antibodies may be completely specific for one or more human CD33 proteins and may not exhibit species or other types of non-human cross-reactivity. The amino acid sequence of an exemplary human CD33 has Genbank accession number NP_{—}075556.1. Exemplary anti-CD33 antibodies include gemtuzumab and lintuzumab, and are also described in U.S. Pat. Nos. 7,557,189 and 8,119,787.

“Anti-CD52 antibody” refers to an antibody that specifically binds to CD52, a GPI anchored cell surface protein expressed on human peripheral lymphocytes and monocytes macrophages but not detected in hematopoietic stem cells. It is also known as CAMPATH-1 antigen. As used herein “CD52” includes, for example, variants, isoforms, and species homologs of human CD52. Anti-CD52 antibodies may, in certain instances, cross-react with CD52 of more than one species. In some embodiments, the antibodies may be completely specific for one or more human CD52 proteins and may not exhibit species or other types of non-human cross-reactivity. The amino acid sequence of an exemplary human CD52 has Genbank/NCBI accession number NP_{—}001794.2. Exemplary anti-CD52 antibodies are described in U.S. Pat. No. 5,846,534 and US patent publication 20120100152.

“Anti-EGFR antibody” refers to an antibody that specifically binds to epidermal growth factor receptor (EFGR), a receptor for epidermal growth factors, also known as Erb1 and Her1 in humans. As used herein “EGFR” includes, for example, variants, isoforms, and species homologs of human EFGR. Anti-EFGR antibodies may, in certain instances, cross-react with EFGR of more than one species. In some embodiments, the antibodies may be completely specific for one or more human EFGR proteins and may not exhibit species or other types of non-human cross-reactivity. The amino acid sequence of an exemplary human EFGR has Genbank/NCBI accession number NM_{—}005228.3 (isoform a) and NM_{—}201283.1 (isoform C). Exemplary anti-EFGR antibodies include cetuximab, and are also described in U.S. Pat. Nos. 5,844,093 and 7,595,378.

“Anti-EphA2 antibody” refers to an antibody that specifically binds to ephrin type A receptor 2, a tyrosine kinase belonging to the ephrin receptor family. As used herein “EphA2” includes, for example, variants, isoforms, and species homologs of human EphA2. Anti-EphA2 antibodies may, in certain instances, cross-react with EphA2 of more than one species. In some embodiments, the antibodies may be completely specific for one or more human EphA2 proteins and may not exhibit species or other types of non-human cross-reactivity. The amino acid sequence of an exemplary human EphA2 has Genbank accession number NP_{—}004422.2. Exemplary anti-EphA2 antibodies are described in U.S. Pat. Nos. 7,402,298, 7,569,672 and 8183357.

“Anti-GD2 antibody” refers to an antibody that specifically binds to ganglioside G2, a disialoganglioside expressed in tumors of neuroectodermal origin. Exemplary anti-GD2 antibodies are described in U.S. Pat. Nos. 6,451,995, 6,872,392 and 7432357.

“Anti-G250 antibody” refers to an antibody that specifically binds to carbonic anhydrase IX (CA-IX) antigen, a zinc metalloenzyme expressed in various tumor cells and also known as tumor associated antigen MN. The cell surface antigen is known to have carbonic anhydrase activity. As used herein “G250” includes, for example, variants, isoforms, and species homologs of human G250. Anti-G250 antibodies may, in certain instances, cross-react with G250 of more than one species. In some embodiments, the antibodies may be completely specific for one or more human G250 proteins and may not exhibit species or other types of non-human cross-reactivity. The amino acid sequence of an exemplary human G250 has Genbank/NCBI accession number NP_{—}001207.2. Exemplary anti-G250 antibodies include Rencarex® and are also described in US patent publications 20090274620 and 20110123537.

“Anti-ErB2 antibody” refers to an antibody that specifically binds to epidermal growth factor receptor 2, also known as Neu and CD340. The human form is designated HER2. As used herein “Erb2” includes, for example, variants, isoforms, and species homologs of human Erb2 (HER2). Anti-Erb2 antibodies may, in certain instances, cross-react with Erb2 of more than one species. In some embodiments, the antibodies may be completely specific for one or more human Erb2 proteins and may not exhibit species or other types of non-human cross-reactivity. The amino acid sequence of an exemplary human Erb2 has Genbank/NCBI accession number NP_{—}001005862.1. Exemplary anti-ErB2 antibodies include trastuzumab and are also described in U.S. Pat. Nos. 5,677,171 and 7,435,797 and US patent publication 20100016556.

“Anti-folate receptor a antibody” refers to an antibody that specifically binds to folate receptor α, also known as FLOR1. The cell surface antigen is known to have carbonic anhydrase activity. As used herein “G250” includes, for example, variants, isoforms, and species homologs of human G250. Anti-G250 antibodies may, in certain instances, cross-react with G250 of more than one species. In some embodiments, the antibodies may be completely specific for one or more human G250 proteins and may not exhibit species or other types of non-human cross-reactivity. The amino acid sequence of an exemplary human G250 has Genbank/NCBI accession number NP_{—}001207.2. Exemplary anti-G250 antibodies include Rencarex® and are also described in US patent publications. 20090274620 and 20110123537.

“Anti-folate receptor β antibody” refers to an antibody that specifically binds to folate receptor β (FR-B), also known as FOLR2, which is a surface antigen expressed in activated macrophages, such as inflammatory macrophages, tumor infiltrating macrophages, and myeloid leukemia cells, and is involved in intracellular transport of folate. As used herein “folate receptor β” includes, for example, variants, isoforms, and species homologs of human folate receptor β. Anti-folate receptor β antibodies may, in certain instances, cross-react with folate receptor β of more than one species. In some embodiments, the antibodies may be completely specific for one or more human folate receptor β proteins and may not exhibit species or other types of non-human cross-reactivity. The amino acid sequence of an exemplary human folate receptor β has Genbank/NCBI accession number NP_{—}000794.3. Exemplary anti-folate receptor β antibodies are described in US patent publication 20080260812.

“Anti-phosphatidylserine antibody” refers to an antibody that specifically binds the phospholipid phosphatidylserine. Antibodies to phosphatidylserine are described in U.S. Pat. Nos. 7,247,303 and 7,422,738.

“Target cell radiolabel release assay” refers to an ADCC assay in which a target cell, for example a cell expressing an surface antigen recognized by an antibody, is loaded with a radiolabel, e.g., ⁵¹Cr, Eu (Europeum), or ³⁵S, and target cell lysis or destruction determined by measuring release of radiolabel.

“Target cell enzyme release assay” refers to an ADCC assay in which a target cell, for example a cell expressing an surface antigen recognized by an antibody, is incubated with the antibody and effector cells expressing Fc receptors and quantitating release of defined enzymes that are natural components of the cells.

“Target cell depletion assay” refers to an ADCC assay measuring the reduction, depletion or killing of cells targeted by an antibody. Target cell depletion assay can be done in vitro, e.g., human B cells used ex vivo with an anti-CD20 antibody and effector cells. In some embodiments, the target cell depletion assay can be in vivo, e.g., by measuring number of B cells in a subject after the administration of an anti-CD20 antibody by withdrawing blood samples, and measuring time-dependent depletion assays over a period of several weeks. Typically MRD-FC is used to measure these populations.

“Target cell repopulation assay” refers to an ADCC assay measuring the recovery (slow or fast) or recover rate of a target cell population following administration of an antibody, e.g., repopulation of B cells following administration of an anti-CD20 antibody. Assays can be in vivo, e.g., by measuring number of specific subsets of B cells repopulating in a subject after the administration of an anti-CD20 antibody by withdrawing blood samples, and measuring time-dependent depletion assays over a period of several weeks. Typically MRD-FC is used to measure these populations in vitro. Faster repopulation is a measure of imminent disease relapse.

“Cell population targeted by an antibody” refers to a cell or group of cells that are specifically recognized by the antibody of interest, and in the context of ADCC, killed or lysed through an ADCC mechanism.

“Clinical trial” refers to an investigation of safety and efficacy of a treatment for a disease or disorder. Typically, clinical trials are carried out to obtain approval from a governmental regulatory agency for marketing a drug.

“Health service payer” refers to an entity that finances or pays for the medical treatment or therapy. A health service payer can include among others, an insurance company, a government entity, or a private company.

### Stratified Medicine, Theragnostics, and Companion Diagnostics

While drugs (e.g., small molecule pharmaceuticals, protein biologics, therapeutic antibodies, etc.) are typically developed to interact with specific biological targets, populations generally show wide variations in response to the drug treatment, due in part, to genetic variations in populations, where the genetic variations affect therapeutic properties of the drug. These genetic variations can affect, among others, the direct biological target of the drug, metabolism of the drug, and/or the biological mechanisms by which the drug mediates its therapeutic effect. Thus, in some instances, a drug may only be effective in individuals or subjects who have a particular genetic or protein variation and ineffective in those individuals who do have the particular genetic or protein variation, and may experience adverse side effects (e.g., increased toxicity). For example, a number of therapeutic antibodies have been developed for treating a variety of diseases including cancers, autoimmune diseases, and inflammatory disorders. However, it is generally acknowledged that many of these antibodies (e.g., rituximab in follicular lymphoma) work more effectively for some patients than others.

In the area of antibody therapeutics, many antibodies have as its therapeutic mechanism, wholly or in part, antibody dependent cell-mediated cytotoxicity (ADCC). ADCC is a process of cell-mediated immunity in which effector cells of the immune system, such as natural killer (NK) cells, macrophages, neutrophils, and eosinophils, kill target cells that have been bound by specific antibodies. Destruction or killing of the target cell can occur through phagocytosis; ADDC-mediated lysis; ADCC-mediated apoptosis; and trogocytosis (antibody-dependent cytotoxicity mediated by polymorphonuclear granulocytes). The posited mechanism of ADCC is the binding of the effector cells to the Fc (constant) portion of the bound antibody through Fc receptors, particularly the Fcγ receptors, present on the effector cells. As such, variations or polymorphisms in the Fc receptor can affect the effectiveness of antibodies that work via the ADCC mechanism. The association between Fc receptor polymorphisms and ADCC has led to use of Fc genotypes for selecting patients for antibody-based therapies, e.g., US patent publication 2006/0008825 and US 20100291549, both of which are incorporated herein by reference. While antibodies may have multiple mechanisms of action, e.g., ADCC, blocking cell signaling or neutralization, ADCC may be a major or contributory mechanism to the therapeutic effects. The contribution by other mechanisms does not preclude or obviate the ADCC mechanism.

Nevertheless, while some patients achieve complete remission and some other patients achieve complete response, a majority of the patients achieve moderate and poor responses to antibody therapy; the disease relapses in a significant majority of the patients. Relapse also occurs for certain number of patients following other types of therapies, such as chemotherapies. Attempts to treat some relapsed patients with maintenance therapy, whether initially treated with antibody therapy or chemotherapy, has been met with limited success (e.g., Vidal et al., Cochrane Database Syst Rev. 2009 Apr. 15; (2); Arce-Salinas et al., Rheumatol Int. 2012 32(5):1245-9. Epub 2011 Jan. 22; Michallet et al., 2011, Curr Opin Oncol. 23(5):449-54; and Tokunaga et al., 2007, Ann Rheum Dis; 66:470-475). The studies do not indicate that the level of ADCC function is effective or available for an antibody maintenance therapy, particularly given the relapsed condition of the subjects. The present disclosure, however, provides for the use of antibody maintenance therapy to treat relapsed patients, where the patients have been stratified based on their genotype by one or more Fc receptor polymorphism to predict their responsiveness to antibody maintenance therapy, thereby allowing selection of patients who would likely benefit from the maintenance treatment. Alternatively, responsiveness can be predicted by measuring the ADCC function or capacity of the patients, thus providing another determinative factor for selecting patients who are likely to have positive treatment outcomes, or conversely, excluding patients who are likely to have a negative treatment outcome, with the maintenance therapy. Predicting responsiveness to antibody maintenance therapy, preferably a priori can also allow selection of various treatment options, including alternatives to antibody therapy if the subject responds poorly to antibody maintenance therapy. This a priori identification and selection of patients who will respond to a therapy has significant commercial and therapeutic advantages, and will be useful to drug developers, physicians, health care payers, pharmacy benefit managers.

Accordingly, as summarized above, the present disclosure provides methods for predicting responsiveness of individuals or subjects with ADCC-treatable diseases or disorders to antibody maintenance therapy, as well as reagents and kits thereof (and devices) for use in practicing the subject methods. The method of predicting responsiveness can be applied to selection of subjects with an ADCC treatable disease for treatment with an antibody maintenance therapy, as well as treating such selected patients with an antibody maintenance. In an ADCC-treatable disease/disorder, the disease/disorder is treatable, as defined herein, in at least some individuals by administering a therapeutic antibody that is specific for a target cell implicated in causing or exacerbating the disease/disorder, and that promotes ADCC of that target cell.

In some embodiments, the method for predicting responsiveness of a subject having an ADCC treatable disease or disorder to an antibody maintenance therapy can comprise:

determining a genotype of the human subject for one or more Fcγ receptor polymorphisms affecting ADCC activity, wherein the Fcγ receptor polymorphism is selected from a FcγRIIA polymorphism and a FcγRIIIA polymorphism; and

predicting a responsiveness of the human subject to an antibody maintenance therapy based upon the determined genotype of the Fcγ receptor polymorphism.

In some embodiments, the method can comprise stratifying the subject having an ADCC treatable disease or disorder to predict responsiveness to antibody maintenance therapy, where the method comprises:

determining a genotype of the human subject for one or more Fcγ receptor polymorphisms affecting ADCC activity, wherein the Fcγ receptor polymorphism is selected from a FcγRIIA polymorphism and a FcγRIIIA polymorphism; and

comparing the determined genotype of the human subject to a reference stratification that relates responsiveness to antibody maintenance therapy for the ADCC treatable disease to genotype of the Fcγ receptor polymorphism.

In some embodiments, the method can comprise stratifying a human subject having an ADCC treatable disease or disorder to predict responsiveness to antibody maintenance therapy, where the method comprises:

comparing a genotype of one or more Fcγ receptor polymorphisms determined for a human subject having an ADCC treatable disease to a reference stratification that relates responsiveness to antibody maintenance therapy for the ADCC treatable disease to genotypes of the Fey receptor polymorphism, wherein the Fcγ receptor polymorphism affects ADCC activity.

In the embodiments herein, the Fcγ receptor polymorphism affects ADCC activity. There are two low affinity Fcγ receptor subclasses, FcγRIIA, and FcγRIIIA, that interact with the Fc portion of antibodies, particularly the IgG subtype. Accordingly, any functional polymorphisms in FcγRIIA, and FcγRIIIA, either alone or in combination, that affects ADCC activity can be used to predict responsiveness, for example by stratification of the subject, to antibody maintenance therapy. In some embodiments, one or more functional polymorphisms in FcγRIIA can be used. In some embodiments, one or more functional polymorphisms in FcγRIIIA can be used. In some embodiments, the Fcγ receptor functional polymorphism is one or more FcγRIIA functional polymorphisms and one or more FcγRIIIA functional polymorphisms, i.e., a combination of polymorphisms in FcγRIIA and the FcγRIIIA.

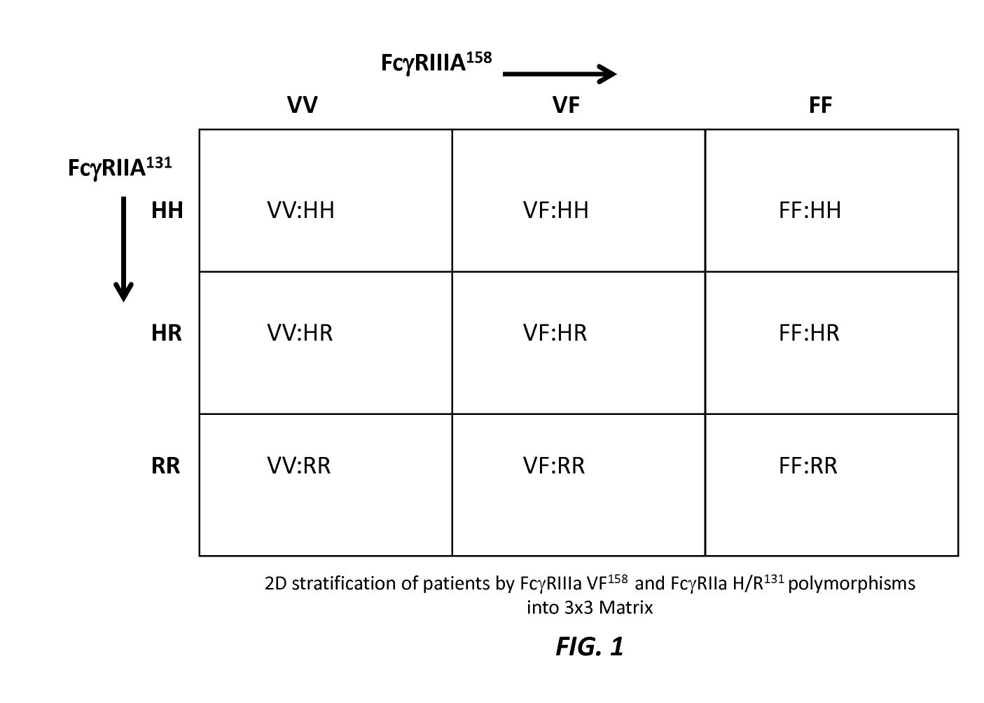

In some embodiments, where two or more different polymorphisms are examined, the genotypes can be stratified multidimensionally, depending on the number of different polymorphisms. For example, two different functional polymorphisms (e.g., 131 of FcγIIA and 158 of FcγIIIA) can be stratified in two dimensions into nine distinct genotype groups, as represented in FIG. 1. Similar analysis can be done for three, four or more different functional polymorphisms. In a multidimensional stratification, outcomes that are closer together can be distinguished as compared to a one dimensional stratification.

In some embodiments, the polymorphism for predicting responsiveness is based on FcγIIA polymorphism at amino acid residue (position) 131. Identified polymorphisms that occur at amino acid position 131 include H¹³¹ (H—histidine) and R¹³¹ (R—arginine). The nucleotide codons encoding the histidine (H) and arginine (R) residues of the polymorphism can be CAT and CGT, respectively. Accordingly, in some embodiments the genotypes for the FcγIIA polymorphism at amino acid position 131 comprise the homozygote H/H¹³¹, heterozygote H/R¹³¹ and homozygote R/R¹³¹.

In some embodiments, the polymorphism for predicting responsiveness is based on FcγIIIA polymorphism at amino acid position 158. Identified polymorphisms that occur at amino acid position 158 include V¹⁵⁸ (V—valine) and F¹⁵⁸ (F—phenylalanine). The nucleotide codons encoding the valine (V) and phenylalanine (F) residues of the polymorphism can be GTT and TTT, respectively. Accordingly, in some embodiments the genotypes for the FcγIIIA polymorphism at amino acid position 158 comprise homozygote V/V¹⁵⁸, heterozygote V/F¹⁵⁸ and homozygote F/F¹⁵⁸.

In some embodiments, where the predicting or stratifying is based on FcγRIIA functional polymorphism at amino acid residue 131 and FcγRIIIA functional polymorphism at amino acid residue 158, the relevant genotype groups are given in Table 1.

TABLE 1

| Group | Genotype |
|---|---|
| I | H/H¹³¹ for FcγRIIA and V/V¹⁵⁸ for FcγRIIIA; |
| II | H/H¹³¹ for FcγRIIA and V/F¹⁵⁸ for FcγRIIIA; |
| III | H/H¹³¹ for FcγRIIA and F/F¹⁵⁸ for FcγRIIIA; |
| IV | H/R¹³¹ for FcγRIIA and V/V¹⁵⁸ for FcγRIIIA; |
| V | H/R¹³¹ for FcγRIIA and V/F¹⁵⁸ for FcγRIIIA; |
| VI | H/R¹³¹ for FcγRIIA and F/F¹⁵⁸ for FcγRIIIA; |
| VII | R/R¹³¹ for FcγRIIA and V/V¹⁵⁸ for FcγRIIIA; |
| VIII | R/R¹³¹ for FcγRIIA and V/F¹⁵⁸ for FcγRIIIA; |
| IX | R/R¹³¹ for FcγRIIA and F/F¹⁵⁸ for FcγRIIIA. |

In some embodiments for stratifying into a responsiveness group, the determined genotype of the subject is compared to a reference stratification that relates or correlates, e.g., classifies responsiveness to the antibody maintenance therapy for the ADCC treatable disease to genotypes of the Fcγ receptor functional polymorphism. As exemplified above, the reference stratification is an established stratification scheme that has stratified a treatment response/clinical outcome-genotype association, with statistically significant differences between the different groups in the stratification. Accordingly, a subject afflicted with an ADCC treatable disease whose genotypes for the Fcγ receptor functional polymorphisms (e.g., FcγIIA and/or FcγIIIA) can be compared to the reference stratification to identify the likelihood of the subject having a particular treatment outcome or clinical outcome, i.e., responsiveness for an antibody maintenance therapy.

The reference stratification, also referred to as a reference index can be prepared for any ADCC treatable disease for a particular antibody therapeutic. In some embodiments, the reference stratification can be prepared by determining the genotype of each subject in plurality of subjects having a ADCC treatable disease or disorder, and determining the treatment outcome or clinical outcome. The statistical significance of the linkage between the genotype and the responsiveness can be determined by standard statistical methods. The treatment outcome or clinical outcome assessments can use diagnostic measures known in the art and typically specific to each disease or disorder. See, e.g., World Health Organization International Classification of Diseases (ICD), e.g., ICD 10 and Merck Manual of Diagnosis and Therapy, Merck Publishing (2011). As further described in the present disclosure, the reference stratification data can be in printed form or stored in a computer memory. In some embodiments, the comparing of the determined genotype of the subject to the reference stratification can be implemented by a computer using methods standard in the art.

Accordingly, the terms “reference” and “control” as used herein refers to a standardized genotype to be used to interpret the genotype of a given patient and assign a prognostic class thereto. The reference or control may be a genotype that is obtained from a cell/tissue known to have the desired phenotype, e.g., responsive phenotype, and therefore may be a positive reference or control genotype. In addition, the reference/control genotype may be from a cell/tissue known to not have the desired phenotype, and therefore be a negative reference/control genotype.

In practicing methods, a subject or patient sample, e.g., cells or collections thereof, e.g., a blood sample or tissue sample, is assayed to predict responsiveness of the patient to an antibody maintenance therapy. For example, a patient with an ADCC-treatable disease who is responsive to antibody maintenance therapy will experience at least a slowing in disease progression; in some instances, at least a cessation of disease progression; in some instances, an improvement in health, i.e. a reversal of disease progression, a loss of disease symptoms, etc. In contrast, a patient with an ADCC-treatable disease that is not responsive to antibody maintenance therapy will not experience at least a slowing in disease progression, or at least a cessation in disease progression, or an improvement in health. In some embodiments in which the induction therapy comprises antibody therapy, responsiveness to an antibody maintenance therapy is responsiveness to maintenance therapy with the same antibody used in the induction therapy. In other embodiments in which the induction therapy comprises antibody therapy, responsiveness to an antibody maintenance therapy is responsiveness to maintenance therapy with an antibody other than that used in the induction therapy.

As further discussed below, any convenient metric available in the art may be used to measure and convey predictions of responsiveness to maintenance therapy. In some embodiments, predictions may be made in terms of progression free survival (PFS), overall survival (OS), relapse-free survival (RFS) and/or event-free survival (EFS), as the terms are defined herein and commonly used in the art, as further discussed below.

In some embodiments, the above-obtained information about the cell/tissue being assayed is employed to diagnose a host, subject or patient with respect to responsiveness to antibody maintenance therapy, as described above. In some embodiments, the above-obtained information is employed to give a refined probability prediction as to whether a subject will or will not respond to a particular therapy.

For example, different combinations of alleles at the FcγRIIA 131 H/R polymorphism and the FcγRIIIA 158 V/F polymorphism are predictive of responsiveness of an individual having an ADCC-treatable disease to antibody maintenance therapy. Essentially, using the combinations of alleles at the FcγRIIA and FcγRIIIA loci, individuals may be stratified, or classified, into one of 9 groups, each group having a different probability of responsiveness to antibody maintenance therapy.

In some embodiments, the predicting responsiveness can be based on the following reference stratification in Table 2 for an ADCC treatable disease (e.g., B non-Hodgkins lymphoma), that associates genotypes of FcγIIA polymorphism at amino acid residue 131 and FcγIIA polymorphism at amino acid residue 158 to responsiveness to antibody maintenance therapy, e.g., with rituximab.

TABLE 2

| Group | Genotype | Responsiveness |
|---|---|---|
| (a) | H/H¹³¹ FcγRIIA and V/V¹⁵⁸ FcγRIIIA | Predictive of excellent |
| | | responsiveness |
| (b) | H/H¹³¹ FcγRIIA and V/F¹⁵⁸ FcγRIIIA; | Predictive of good |
| | H/H¹³¹ FcγRIIA and F/F¹⁵⁸ FcγRIIIA; | responsiveness |
| | H/R¹³¹ FcγRIIA and V/V¹⁵⁸ FcγRIIIA; | |
| | R/R¹³¹ FcγRIIA and V/V¹⁵⁸ FcγRIIIA; | |
| (c) | H/R¹³¹ FcγRIIA and V/F¹⁵⁸ FcγRIIIA | Predictive of moderate |
| | | responsiveness; |
| (d) | H/R¹³¹ FcγRIIA and F/F¹⁵⁸ FcγRIIIA; | Predictive of weak |
| | R/R¹³¹ FcγRIIA and V/F¹⁵⁸ FcγRIIIA | responsiveness |
| (e) | R/R¹³¹ FcγRIIA and F/F¹⁵⁸ FcγRIIIA | Predictive of poor |
| | | responsiveness. |

In reference to Table 2, an individual with an ADCC-treatable disease, e.g., B-NHL, who has two H alleles at amino acid residue 131 of FcγRIIA and two V alleles at amino acid residue 158 of FcγRIIIA (i.e., “H/H¹³¹ FcγRIIA, V/V¹⁵⁸ FcγRIIIA”) is predicted to have an excellent response to antibody maintenance therapy, e.g., maintenance therapy with rituximab, i.e., a 90% chance or more of progression free survival 3 years or more. An individual with an ADCC-treatable disease, e.g., B-NHL, having two H alleles at amino acid residue 131 of FcγRIIA and either one or two F alleles at amino acid residue 158 of FcγRIIIA (i.e., “H/H¹³¹ FcγRIIA, V/F¹⁵⁸ FcγRIIIA” or “H/H¹³¹ FcγRIIA, F/F¹⁵⁸ FcγRIIIA”), or having two V alleles at amino acid residue 158 of FcγRIIIA and either one or two R alleles at amino acid residue 131 of FcγRIIA (i.e., “H/R¹³¹ FcγRIIA, V/V¹⁵⁸ FcγRIIIA” or “R/R¹³¹ FcγRIIA, V/V¹⁵⁸ FcγRIIIA”) is predicted to have a good responsiveness to antibody maintenance therapy, e.g., maintenance therapy with rituximab, i.e. an 80% chance or more of progression free survival 3 years or more. An individual with an ADCC-treatable disease, e.g., NHL, who is heterozygous at both residues 131 of FcγRIIA and residue 158 of FcγRIIIA (i.e., “H/R¹³¹ FcγRIIA, V/F¹⁵⁸ FcγRIIIA”) is predicted to have a moderate responsiveness to antibody maintenance therapy, e.g., maintenance therapy with rituximab, i.e. a 60% chance or more of progression free survival 3 years or more. An individual with an ADCC-treatable disease, e.g., NHL, having two R alleles at amino acid residue 131 of FcγRIIA and one V and one F allele at amino acid residue 158 of FcγRIIIA, or having two F alleles at amino acid residue 158 of FcγRIIIA and one H and one R allele at amino acid residue 131 of FcγRIIA (i.e., “R/R¹³¹ FcγRIIA, V/F¹⁵⁸ FcγRIIIA” or “H/R¹³¹ FcγRIIA, F/F¹⁵⁸ FcγRIIIA genotype”) is predicted to have a weak responsiveness to antibody maintenance therapy, e.g., maintenance therapy with rituximab, i.e., a 40-50% chance or more of progression free survival 3 years or more. Finally, an individual with an ADCC-treatable disease, e.g., NHL, having two R alleles at amino acid residue 131 of FcγRIIA and two F alleles at FcγRIIIA (i.e., “R/R¹³¹ FcγRIIA, F/F¹⁵⁸ FcγRIIIA”) is predicted to have a poor responsiveness, i.e. to be non-responsive, to antibody maintenance therapy, e.g., maintenance therapy with rituximab, i.e., a 25% chance or less of progression free survival 3 years or more.

In some embodiments, excellent responders may exhibit, e.g., at least about 85%, 90%, or higher mean or median response rates (or better than about 85 percentile measure of outcome among the unstratified population); very good responders may exhibit lesser measures of responsiveness, e.g., at least about 70%, 75%, or 80% response rates (or from about top 75th percentile to 85th of outcomes); good responders may have better than average response rates, e.g., at least about 55%, 60%, or 65% response rates (or from about top 55th percentile to 75th of outcomes); moderate responders will typically have near average response rates, e.g., in the range of about 45%, 50%, or 55% response rates (or from about 45th to 55th percentile of outcomes); below average responders may have lower response rates, e.g., below about 45%, 35%, or 30% (or from about 25th percentile to about 45th percentile of outcomes); very poor responders may have even lower response rates, e.g., below about 25%, 20%, or 15%, and non-responders may have even lower response rates, e.g., less than about 12%, 10%, or 5%.

In some embodiments, the average overall response rates to treatment for overall unstratified population will be in the 40% to 60% range. The above and below average responder subsets will preferably have at least about 7-15% better and lower relative mean or median responsiveness measures, respectively, and the good and poor responders will preferably have at least another 7-15% better and lower mean or median responsiveness measures, respectively. The very good and very poor responders will have correspondingly better and worse mean or median responsiveness measures, and the excellent and non-responders even more extreme. How many different stratification categories are used will depend largely upon the dispersion of the responsiveness measures across categories of treatment response, and the variation of individual responsiveness measures within each category of treatment response. In some embodiments, the range of responsiveness across the categories will range from less than about 10% to at least about 90%.

In some embodiments, the patients may be stratified by strata of percentile responsiveness ranges. Thus, the highest may be the top 15 percentile stratum of response, the next the second top 15 percentile stratum, etc., down to the lowest category of the bottom 15 percentile stratum, providing six strata of responsiveness. Improvement of responsiveness may be moving from one stratum to a higher stratum, preferably two or more.

In some embodiments, after the 3×3 matrix of (nine) genotype groups is related to the maintenance treatment response, treatment response categories can be pooled where appropriate (e.g., with similar or indistinguishable response measures and dispersion) to provide a reference with fewer than the 3×3 (or nine) categories of (maintenance therapy) treatment response (CTR). The simplified index with fewer CTR than genotype groups might be a second derived reference index. However, different categories of treatment response should be combined after it is determined that they are, in fact, sufficiently similar to make such combination desirable. Pooling disparate categories of treatment response into one larger category corresponding to multiple genotype groups may reduce the predictability of treatment outcome, e.g., increasing variation or dispersion of prediction among individuals in the genotype groups. The number of different categories of maintenance treatment response is selected to be practical and useful for stratification purposes. If there is wide range of responsiveness among different genotype groups, more stratification of categories (9, 8, 7, 6, or 5 categories) may be retained, e.g., relatively very high (or excellent) responders, high responders, intermediate responders, low responders, very low responders. Too few categories (e.g., only 2, 3, or 4 categories) may fail to provide useful discrimination between different subsets, e.g., where all categories are pooled into a population where genotype group stratification is ignored. Of particular interest are reference indices which distinguish specifically H/R¹³¹ from R/R¹³¹ on the 2A dimension, and/or V/F¹⁵⁸ from F/F¹⁵⁸ on the 3A dimension, which might distinguish all three genotypes in each dimension.

In some embodiments, pooling or combining different genotype groups can be done when the responses are similar. The genotype groups which will be combined will typically share one or more allele. Thus, in the groups I through IX as designated above, typically two or more adjacent genotype groups can be combined. Particularly useful combined sets would include one or more of [I+II], [I+IV], [IX+VIII], [IX+VI], [I+II+IV], [I+II+III], [IV+V+VI], [VII+VIII+IX], [I+IV+VII], [II+V+VIII], [III+VI+IX], [I+II+IV], [IX+VIII+VI], [V+VI+VIII+IX], [I+II+IV+V], [I+II+III+IV], [I+II+IV+VII], [I+II+III+IV+V], [I+II+IV+V+VII], or [I+II+III+IV+V+VII]. In various other embodiments, preferred combinations of genotype groups would include one or more of, e.g., III, [II+III], [III+VI], [II+III+VI], [II+III+V+VI], [I+II+III+VI], [II+III+VI+IX], [I+II+III+VI+IX], [I+II+III+V+VI], [II+III+V+VI+IX], [I+II+III+V+VI+IX], VII, [IV+VII], [VII+VIII], [IV+VII+VIII], [IV+V+VII+VIII], [I+IV+VII+VIII], [IV+VII+VIII+IX], [I+IV+VII+VIII+IX], [I+IV+V+VII+VIII], [IV+V+VII+VIII+IX], [I+IV+V+VII+VIII+IX], IX, [VI+IX], [VIII+IX], [VI+VIII+IX], [V+VI+VIII+IX], [III+VI+VIII+IX], [VI+VII+VIII+IX], [III+VI+VII+VIII+IX], [III+V+VI+VIII+IX], [V+VI+VII+VIII+IX], or [III+V+VI+VII+VIII+IX]. In other embodiments, any combination of 2, 3, 4, 5, or more genotype groups may be clustered together. Patent applications WO/2007/127936 and U.S. Ser. No. 12/298,661 describe related subject matter, and both are incorporated herein by reference.

In some embodiments, a reference stratification or reference index relating genotype group to categories of antibody maintenance treatment response can be used in both directions. It can be used to predict the responsiveness to maintenance treatment based on genotype at the relevant positions. This will be very useful for the patient and treating doctor, to provide means to arrive at likely response to alternative treatments. Conversely, for a given responsiveness to maintenance treatment, one can identify genotypes of patients which should achieve such response. Thus, a drug developer or treatment payer may identify which patients are likely to response as indicated by the reference. A drug developer may include only highly responsive genotype target patients into a trial, and/or may exclude those who do not respond to the treatment. Alternatively, for those who respond poorly, additional or alternative treatment strategies may be applied. In other embodiments, those who would respond poorly are not treated with an available treatment with low efficacy for those patients. For a payer, particular treatment strategies might be paid only for patients whose genotypes indicate good responsiveness. Alternatively, those patients whose responsiveness is low may be directed immediately to alternative treatment strategies which have higher success rates. Where the maintenance treatment may improve response after a period of maintenance therapy for certain genotype groups, recognizing which patients will respond can provide many benefits, both to the payer and to the patient.

In the embodiments herein, the subject, preferably a human subject, has had or will have induction therapy. In some embodiments, the induction therapy can comprise chemotherapy. In some embodiments the induction therapy can comprise antibody therapy. In some embodiments, the subject has previously received or is receiving antibody maintenance for the disease or disorder.

As will be apparent to the skilled artisan in view of the teachings provided herein and given the general conservation of the Fc portion of antibodies, many therapeutic antibodies, particularly those with IgG isotypes, are expected to be influenced similarly by polymorphisms at FcγIIA and/or FcγIIIA, including the polymorphisms at amino acid position 131 of FcγIIA and amino acid position 158 of FcγIIIA. Accordingly, the genotype and the predicted responsiveness in Table 2 can be applied to many antibodies, particularly where the Fc region is human IgG₁, that have ADCC as a therapeutic mechanism across many different diseases and disorders, and therefore applicable to the various methods described in the present disclosure.

On the other hand, ADCC-dependent antibody therapies use primarily FcγRIIA receptor (and not FcγRIIIA receptor) if the Fc region is a human IgG₂ (e.g., panitumumab; Schneider-Merck, et al. (2010) J. Immunol. 184:512-520). In such cases, FcγRIIA H/H¹³¹ is the preferred polymorphism. In yet another instance, if an antibody therapy uses a murine IgG₃ Fc framework, FcγRIIA is the preferred receptor, and in fact preferentially uses R/R¹³¹ polymorphism (Cheung, et al. (2006) J. Clin. Oncol. 24:2885-90). Thus, a reference stratification of genotype-responsiveness association can be prepared for each antibody class, or each specific antibody for an ADCC treatable disease.

As an example for demonstrating FcγRIIIA/FcγRIIA relevance to response to antibody maintenance therapy, follicular lymphoma is described below in Example 7.

Other studies on antibody treatment in oncology include Hochster, et al. (2009) J. Clin. Oncol. 27:1607-14 (advanced indolent lymphoma); Hainsworth, et al. (2005) J. Clin. Oncol. 23:1088-95 (indolent B-NHL); and Weng and Levy (2003) J. Clin. Oncol. 21:3940-47 (follicular lymphoma), among others. These are cited, in part, to indicate different criteria and designs for performing a clinical study directed to similar indications, but also to show that these studies are neither unusual nor impractical. The studies would preferably be modified to incorporate more detailed evaluation measures, and to apply principles of antibody maintenance therapy which consider the ADCC mechanisms described herein. In particular, the antibody maintenance therapy courses would typically avoid any aspects which would decrease ADCC function in the patient when the antibody is mediating the ADCC, and perhaps incorporate any features which would improve ADCC function of the administered antibody.

Similar strategies may be applied in other ADCC-treatable diseases, e.g., in autoimmune disorders. Examples of such studies in rheumatoid arthritis include Mease, et al. (2010) J. Rheumatol. 37:917-927 (SUNRISE); Keystone, et al. (2007) Arthritis Rheum, 56:3896-908; Emery, et al. (2010) Ann. Rheum. Dis. 69:1629-635 (SERENE); and Rubbert-Roth, et al. (2010) Rheumatology (Oxford) 49:1683-1693 (MIRROR).

The aforementioned studies serve to exemplify the considerations and details of a trial directed to rheumatoid arthritis. If samples of patients were archived, genotypes of FcγRIIIA and FcγRIIA polymorphisms may be determined to stratify the patients whose outcomes to treatment are now known. Where the treatment protocols applied approach an optimal antibody maintenance treatment as described herein, some indication of the success of proper antibody maintenance treatment regimen can be determined. As above, in follicular lymphoma, a retrospective analysis of data may be generated with genotype information. Prospectively, similar studies may be performed testing a particular antibody maintenance therapy regimen and establish the dependence of response to antibody maintenance therapy on the FcγRIIIA and FcγRIIA polymorphism genotypes.

The data are evaluated to determine if the treatment responses of individuals can be related to the genotype groups, as described above for the two separate antibody maintenance and antibody retreatment arms. In particular, the data are queried as to whether the response rates of the high ADCC function genotype groups (e.g., genotype groups I, II, III, IV, and VII, or subsets among them) are distinguishable from response rates of low ADCC function genotypes (e.g., genotype groups IX, VIII, VI, and V, or subsets among them). Where statistically significant differences are observed between the genotypes, stratification of patients according to genotype may provide useful predictions for responsiveness to the antibody maintenance therapy. In particular, certain genotype groups may be converted with sustained maintenance therapy into relatively higher response outcomes.

In some embodiments, while genotype evaluation results may be reported separately from therapy recommendations, the interpretation of genotype results will often be provided in report describing preferred or standard treatment options. Thus for the various methods of the present disclosure, the genotype information, the stratification, the selection/exclusion of subjects for therapy, the predicted treatment outcome, and the treatment options, as further discussed in the present disclosure can be reported in electronic, web-based, or paper form to the human subject, a health care payer, third party payer, a health care provider, a physician, a pharmacy benefits manager or government office.

As described above, the methods for predicting responsive can be applied to the selection of subjects who are likely to respond positively to antibody maintenance therapy. Conversely identification of subjects who respond poorly provides an opportunity to choose alternative treatments that could produce better treatment outcomes than the said antibody maintenance therapy. In addition to the benefit for the patient, the ability to select subjects who are likely to have a more favorable treatment outcome provides many advantages to drug developers, hospitals, physicians, health service payers, and government run health care services.

Accordingly, in some embodiments, a method for selecting a human subject having an antibody dependent cell-mediated cytotoxicity (ADCC)-treatable disease for treatment with an antibody maintenance therapy comprises:

determining a genotype of the human subject for one or more Fcγ receptor polymorphisms affecting ADCC activity, wherein the Fcγ receptor polymorphism is selected from a FcγRIIA polymorphism and a FcγRIIIA polymorphism; and

selecting or excluding the human subject for treatment with the antibody maintenance therapy based on the determined genotype of the Fcγ receptor polymorphism.

In some embodiments, a method for selecting a human subject having an antibody dependent cell mediated cytotoxicity (ADCC) treatable disease for treatment with an antibody maintenance therapy comprises:

determining a genotype of the human subject for one or more Fcγ receptor polymorphisms affecting ADCC activity, wherein the Fcγ receptor polymorphism is selected from a FcγRIIA polymorphism and a FcγRIIIA polymorphism; and

stratifying the human subject into a responsiveness group based on the determined genotype; and

selecting or excluding the human subject for antibody maintenance therapy based on the stratification.

As noted above, stratifying of the subject into a responsiveness group is carried out by comparing the determined genotype of the human subject to a reference stratification that relates responsiveness to antibody maintenance therapy for the ADCC treatable disease to genotypes of the Fcγ receptor functional polymorphisms.

In the methods of selecting or stratifying a subject for antibody maintenance therapy, the Fcγ receptor polymorphism used can be one or more FcγRIIA functional polymorphisms and/or one or more FcγRIIIA functional polymorphisms described above, particularly the amino acid position 131 of FcγRIIA and the amino acid position 158 of FcγRIIIA.

Accordingly, in some embodiments, the selection of a subject for antibody maintenance is based on the genotype groups described in Table 1 and the stratification of genotype-responsiveness provided in Table 2, above.

Accordingly, in some embodiments, the selection of a subject for antibody maintenance therapy can be based on the stratification of the subject into genotype group (a), (b) or (c) of Table 2. In some embodiments, the selection of a subject for antibody maintenance therapy can be based on the stratification of the subject into genotype group (b) or (c).

Conversely, in some embodiments, subjects who respond weakly or poorly to antibody maintenance therapy can be excluded from treatment. In some embodiments, exclusion of a subject in group (d) or (e), particularly (e).

As further described below, the method of treating can further comprise measuring the level of ADCC capacity or function in the subject, providing another independent criterion or metric for selecting subjects who will likely have a positive treatment outcome for the antibody maintenance therapy. Examples include selective or non-selective depletion of specific subsets of B-cells, inflammatory macrophages, tumor infiltrating macrophages, inflammatory NKT-cells etc. Selective repopulation of specific subsets of B-cells is yet another example of measurement of ADCC function.

In some embodiments, the present disclosure further provides methods of treating subjects with an ADCC treatable disease or disorder based on selection of a subject who is likely to have positive treatment outcomes. In some embodiments, a method of treating a human subject having an antibody dependent cell-mediated cytotoxicity (ADCC)-treatable disease or disorder with an antibody maintenance therapy comprises:

determining a genotype of the subject for one or more Fcγ receptor functional polymorphisms affecting ADCC activity, wherein the Fcγ receptor functional polymorphism is selected from a FcγRIIA functional polymorphism and a FcγRIIIA functional polymorphism;

stratifying the human subject into a responsiveness group based on the determined genotype, and selecting or excluding the human subject for antibody maintenance therapy based on the stratification; and

administering to the selected human subject the antibody maintenance therapy regimen.

In some embodiments, a method of treating a subject having an antibody dependent cell-mediated cytotoxicity (ADCC)-treatable disease or disorder with an antibody maintenance therapy comprises:

selecting or excluding the human subject for antibody maintenance therapy by stratifying the human subject into a responsiveness group based on a determined genotype of the human subject for one or more Fcγ receptor functional polymorphisms affecting ADCC activity, wherein the Fcγ receptor functional polymorphisms is selected from a FcγRIIA functional polymorphism and a FcγRIIIA functional polymorphism; and

administering to the selected human subject the antibody maintenance therapy regimen.

As described herein, stratification of the subject into a responsiveness group is carried out by comparing the determined genotype of the human subject to a reference stratification that relates responsiveness to antibody maintenance therapy for the ADCC treatable disease to genotypes of the Fcγ receptor polymorphism.

The Fcγ polymorphism affecting ADCC activity can be one or more FcγRIIA polymorphisms and/or one or more FcγRIIIA polymorphisms described above, particularly the amino acid position 131 of FcγRIIA and the amino acid position 158 of FcγRIIIA.

Accordingly, in some embodiments the stratification in the treatment methods can be based on the genotype groups described in Table 1 and the subject selected based on the corresponding stratification of genotype-responsiveness provided in Table 2, above.

In some embodiments, administration of antibody maintenance therapy can be based on the stratification of a subject into genotype group (a), (b) or (c) of Table 2. In some embodiments, administration of antibody maintenance therapy can be based on the stratification of a subject into genotype group (b) or (c).

In some embodiments, stratification of subjects into genotype group (d) or (e) is excluded from administration of antibody maintenance therapy particularly (e).

As further described below, the method of treating can further comprise measuring the level of ADCC capacity or function in the subject, providing another independent criterion or metric for treating subjects who will likely have a positive treatment outcome for the antibody maintenance therapy. Examples include selective or non-selective depletion of specific subsets of B-cells, inflammatory macrophages, tumor infiltrating macrophages, inflammatory NKT-cells etc. Selective repopulation of specific subsets of B-cells is yet another example of measurement of ADCC function.

In the methods herein, a wide variety of types of diseases and disorders comprise ADCC-treatable disorders, i.e. are treatable by administering antibodies that promote ADCC of target cells. In some embodiments, the ADCC treatable disease or disorder is selected from a neoplastic disease, an autoimmune disease, an inflammatory disorder, a microbial infection, or allograft rejection.

In some embodiments, the ADCC treatable disease comprises a neoplastic disease, hyperproliferative disorders, or malignancies, which are characterized by unregulated cell growth. Neoplastic diseases include, among others, acute lymphoblastic leukemia (ALL); acute myeloid leukemia (AML); bladder cancer; bone cancer; bowel cancer; brain tumors; breast cancer; cancer of unknown primary; carcinoid; cervical cancer; choriocarcinoma; chronic lymphocytic leukemia (CLL); chronic myeloid leukemia (CML); colon cancer; colorectal cancer; endometrial cancer; eye cancer; gallbladder cancer; gastric cancer; gestational trophoblastic tumors (GTT); hairy cell leukemia; head and neck cancer; Hodgkin's lymphoma; kidney cancer; laryngeal cancer; leukemia; liver cancer; lung cancer; non-small cell lung cancer; lymphoma; melanoma skin cancer; molar pregnancy; mouth and oropharyngeal cancer; myeloma; nasal and sinus cancers; nasopharyngeal cancer; B-cell non Hodgkin's lymphoma (B-NHL); neuroblastoma; esophageal cancer; ovarian cancer; pancreatic cancer; penile cancer; prostate cancer; rectal cancer; salivary gland cancer; skin cancer (non melanoma); soft tissue sarcoma; stomach cancer; testicular cancer; thyroid cancer; unknown primary cancer; uterine cancer; vaginal cancer; vulval cancer; and the like.

In some embodiments, one class of neoplastic diseases for which a number of ADCC-based therapies have been developed is the hematological malignancies, e.g., B-cell malignancies, e.g., non-Hodgkin's Lymphomas (B-NHL). B-cell malignancies are those disorders that derive from cells in the B cell lineage, typically including hematopoietic progenitor cells expressing B lineage markers, pro-B cells, pre-B cells, B-cells and memory B cells; and that express markers typically found on such B lineage cells. The B-NHL are a variety of B-cell neoplasms, and include precursor B-lymphoblastic leukemia/lymphoma; peripheral B-cell neoplasms, e.g. B-cell chronic lymphocytic leukemia; prolymphocytic leukemia; small lymphocytic lymphoma; mantle cell lymphoma; follicular lymphoma; marginal zone B-cell lymphoma; splenic marginal zone lymphoma; hairy cell leukemia; diffuse large B-cell lymphoma; T-cell rich B-cell lymphoma, Burkitt's lymphoma; high-grade B-cell lymphoma, (Burkitt-like); etc. Markers that are specifically found on B cells that may be used as target antigens for ADCC-based therapies include CD45R, which is an exon-specific epitope found on essentially all B cells, and is maintained throughout B cell development (Coffman et al. (1982) Immunol. Rev. 69:5-23); CD19, CD20, CD22, and CD23, which are selectively expressed on B cells and have been associated with B cell malignancies (Kalil and Cheson (2000) Drugs Aging 16(1):9-27; U.S. Pat. No. 6,183,744, herein incorporated by reference); surface immunoglobulin, including epitopes present on the constant regions or idiotypic determinants, which have been utilized in immunotherapy (Caspar et al. (1997) Blood 90(9):3699-706); and the MB-1 antigen, found on all normal immunoglobulin (Ig)-expressing cells, but not on T cells, thymocytes, granulocytes, or platelets, and expressed by virtually all Ig-expressing B cell tumors (Link et al. (1986) J Immunol 137(9):3013-8). Other B cell antigens of interest known to be expressed, for example, on B non-Hodgkin's lymphomas, are Muc-1; B5; BB1; and T9 (Freedman et al. (1987) Leukemia 1(1):9-15). Of particular interest is the CD20 antigen, a human B cell marker that is expressed during early pre-B cell development and remains until plasma cell differentiation. U.S. Pat. No. 5,736,137, herein incorporated by reference, describes the chimeric antibody “C2B8” (also known as RITUXAN®, rituximab, MABTHERA®) that binds the CD20 antigen and its use to treat B cell lymphoma.

ADCC-based therapies have also been developed for solid tumors, e.g., colorectal cancer, non-small cell lung cancer, small cell lung cancer, ovarian cancer, breast cancer, head and neck cancer, renal cell carcinoma, and the like. Table 3 below includes a number of ADCC-treatable neoplastic diseases, the antibody therapies used to treat these diseases, and the antigens—CD52, VEGF, CD30, EGFR, CD22, CD33, CD20, CTLA4, CD2, CD25, EphA2, G25, ErbB2, phosphatidylserine, and HER2—that these antibodies target.

In some embodiments, the ADCC-treatable disease or disorder is an autoimmune disease. Autoimmune diseases are diseases characterized by an overactive immune response of the body against substances and tissues normally present in the body. Examples of autoimmune diseases include, among others, agammaglobulinemia, amyotrophic lateral sclerosis, ankylosing Spondylitis, autoimmune cardiomyopathy, autoimmune hemolytic anemia, autoimmune lymphoproliferative syndrome, autoimmune peripheral neuropathy, autoimmune pancreatitis, autoimmune uveitis, Behcet's disease, Berger's disease, celiac disease, Chagas disease, chronic obstructive pulmonary disease, Churg-Strauss syndrome, Crohn's disease, colitis, diabetes mellitus type 1, discoid lupus erythematosus, Goodpasture's syndrome, Graves' disease, Guillain-Barré syndrome (GBS), idiopathic pulmonary fibrosis, idiopathic thrombocytopenic purpura, IgA nephropathy, inclusion body myositis, chronic inflammatory demyelinating polyneuropathy, Kawasaki's disease, mixed connective tissue disease, multiple sclerosis, myasthenia gravis, hemolytic disease of the newborn (HDN), pemphigus vulgaris, polymyositis, progressive inflammatory neuropathy, psoriasis, psoriatic arthritis, rheumatoid arthritis (RA), juvenile RA, Sjögren's syndrome, systemic lupus erythematosus, lupus nephritis, systemic vasculitides, and Wegener's granulomatosis, B-cell lymphoproliferative disorders and malignancies due to HCV infection of the B-cells, and the like.

In some embodiments, the ADCC treatable disease is an inflammatory disease. In many instances, inflammatory diseases or disorders occur in the context of autoimmune diseases. Exemplary inflammatory diseases include, among others, Crohn's disease, ulcerative colitis, inflammatory bowel disease, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, osteoarthritis, multiple sclerosis, systemic lupus erythematosus, diabetes mellitus, glomerulonephritis, and the like.

In some embodiments, the ADCC-treatable disease or disorder is a microbial infection by a pathogen, including viruses, bacteria, fungi, protozoa, and multicellular parasites. Microbial infections of interest include hepatitis C virus, HIV, malaria, tuberculosis, and other ADCC mechanism infectious processes, or phagocytosis related mechanisms.

In some embodiments, the ADCC-treatable disease or disorder is an allograft, i.e. transplant, rejection. Organs that are typically transplanted include heart, kidneys, liver, lungs, pancreas, intestine, and thymus. Antibodies used to treat organ rejection can be targeted to markers expressed on cells that mediate allograft rejection, for example CD25 (anti-CD25) and CD3 (anti-CD3).

An antibody for any of the methods in the present disclosure, e.g., predicting responsiveness, selection, treatment, etc., is used in the broadest sense, as defined herein, so long as they exhibit the desired biological activity (e.g., binding to target and mediating ADCC). Antibodies for the purposes herein include, among others, chimeric, humanized or fully human antibodies. In some aspects, a combination of one or more antibodies with different specificities, either for epitopes of a single antigen, or for multiple antigens, may be used.

The appropriate antibody can be chosen by the skilled artisan in view of the ADCC treatable disease or condition and the target of the antibody. For example, in some embodiments, where the ADCC treatable disease is a neoplastic disease, the antibodies can comprise an anti-CD19 antibody, anti-CD20 antibody, anti-CD22, anti-CD25 antibody, anti-CD30 antibody, anti-CD33 antibody, anti-CD52 antibody, anti-EGFR, anti-EphA2 antibody, anti-GD2 antibody, anti-G250 antibody, anti-ErB2 antibody, anti-folate receptor a antibody, anti-folate receptor β antibody, or anti-phosphatidylserine antibody, or combinations thereof, depending on the specific neoplastic disease. Exemplary antibodies for the foregoing are described herein, including in the Definition section of the present disclosure.

For the specific neoplastic disease of B non-Hodgkin's lymphoma (NHL), the antibody can comprise anti-CD20 antibody. Exemplary anti-CD20 antibodies can be selected from, among others, rituximab, ofatumumab, ibritumomab, tositumomab, veltuzumab, and obinutuzumab.

In some embodiments, where the ADCC treatable disorder is an autoimmune disease, the antibody for maintenance therapy can comprise, among others, an anti-CD29 or anti-CD20 antibody. Exemplary anti-CD20 antibodies that can be used to treat autoimmune diseases include those described for neoplastic diseases above. For the autoimmune disease systemic lupus erythematosus, an anti-CD20 antibody, such as rituximab or veltuzumab can be selected.

In some embodiments, where the ADCC treatable disorder is an inflammatory disease, the antibody for maintenance therapy can comprise, among others, an anti-folate receptor β antibody, an anti-CD20 antibody (or other B cell targeting antibodies), or an anti-iNKT (invariant natural killer T cell) antibody, such as NKTT120. The folate receptor β is expressed on inflammatory macrophages, e.g., FRβ⁺ macrophages (Choudhury, et al., (2005) Nat. Clin. Prac. Cardiovasc. Med. 2:309-15) implicated in atherosclerotic plaque formation, while CD20 is found on B cells, which is implicated in obesity-associated insulin resistance. In some embodiments, FRβ⁺ macrophages can be selectively depleted, preferably by an ADCC-dependent, anti-FRβ⁺ antibody therapy. The NKTT120 antibody targets iNKT cells, which are increased in chronic inflammation associated with sickle cell disease.

When used for treatment, the antibodies can be administered by methods well known in the art. These include administration by subcutaneous, intravenous, intramuscular, intradermal, transdermal, intratumoral, peritumoral, intrathecal, inhalation, or other routes.

Table 3 below also includes examples of antibody therapies used to treat various diseases, and the antigens that these antibodies target. It is to be understood that each of the exemplary antibodies in Table 3 can be used in the methods, kits, and devices, as described herein.

TABLE 3

| Antibody | | |
|---|---|---|
| (Trade name) | Target antigen | Disease indication |
| Abciximab | glycoprotein IIb/IIIa | Cardiovascular disease |
| (REOPRO ®) | | |
| Adalimumab | TNF-α | Autoimmune disorders, e.g., |
| (HUMIRA ®) | | rheumatoid arthritis, psoriatic |
| | | arthritis, ankylosing spondylitis, |
| | | Crohn's disease, moderate to |
| | | severe chronic psoriasis and |
| | | juvenile idiopathic arthritis. |
| Alemtuzumab | CD52 | B cell malignancies, e.g., chronic |
| (CAMPATH ®) | | lymphocytic leukemia |
| Basiliximab | IL-2Rα receptor (CD25) | Transplant rejection |
| (SIMULECT ®) | | |
| Bavituximab ®) | phosphatidylserine | Breast cancer |
| Belimumab | B-cell activating factor | Autoimmune disorders, e.g., |
| (BENLYSTA ®) | | Systemic lupus erythematosus |
| Bevacizumab | Vascular endothelial | Colorectal cancer, Age related |
| (AVASTIN ®) | growth factor (VEGF) | macular degeneration |
| Brentuximab vedotin | CD30 | Anaplastic large cell lymphoma |
| (ADCETRIS ®) | | (ALCL), Hodgkin's lymphoma |
| Canakinumab | IL-1β | Cryopyrin-associated periodic |
| (ILARIS ®) | | syndromes (CAPS) |
| Certolizumab pegol | TNF-α | Autoimmune disorders, e.g., |
| (CIMZIA ®) | | Crohn's disease |
| Cetuximab | epidermal growth factor | colorectal cancer, Head and neck |
| (ERBITUX ®) | receptor (EGFR) | cancer |
| Daclizumab | IL-2Rα receptor (CD25) | Transplant rejection |
| (ZENAPAX ®) | | |
| Denosumab | RANK Ligand | Postmenopausal osteoporosis, |
| (PROLIA ®), XGEVA ®) | | Solid tumor's bony metasteses |
| Eculizumab | Complement system protein | Paroxysmal nocturnal |
| (SOLIRIS ®) | C5 | hemoglobinuria |
| Efalizumab | CD11a | Autoimmune disorders, e.g., |
| (RAPTIVA ®) | | psoriasis |
| Epratuzumab | CD22 | B cell malignancies, e.g., hairy |
| (LYMPHOCIDE ®) | | cell leukemia |
| Edrecolomab | 17-1A | colon cancer, rectal cancer |
| (PANOREX ®) | | |
| Gemtuzumab | CD33 | acute myelogenous leukemia |
| (MYLOTARG ®) | | |
| Golimumab | TNF-α | Autoimmune disorders, e.g., |
| (SIMPONI ®) | | rheumatoid arthritis, Psoriatic |
| | | arthritis, Ankylosing spondylitis |
| Ibritumomab tiuxetan | CD20 | B cell malignancies, e.g., B non- |
| (ZEVALIN ®) | | Hodgkin's lymphoma |
| Infliximab | TNF-α | Autoimmune disorders, e.g., |
| (REMICADE ®) | | psoriasis, Crohn's disease, |
| | | ankylosing spondylitis, psoriatic |
| | | arthritis, rheumatoid arthritis, |
| | | ulcerative colitis |
| Ipilimumab, MDX-101 | CTLA-4 | Melanoma, non-small cell lung |
| (YERVOY ®) | | carcinoma (NSCLC), small cell |
| | | lung cancer (SCLC), metastatic |
| | | hormone-refractory prostate |
| | | cancer |
| Muromonab-CD3 | CD3 | Transplant rejection |
| (ORTHOCLONE | | |
| OKT3 ®) | | |
| Natalizumab | alpha-4 (α4) integrin | Autoimmune disorders, e.g., |
| (TYSABRI ®) | | multiple sclerosis and Crohn's |
| | | disease |
| Ofatumumab | CD20 | B cell malignancies, e.g., chronic |
| (ARZERRA ®) | | lymphocytic leukemia |
| Omalizumab | immunoglobulin E (IgE) | allergy-related asthma |
| (XOLAIR ®) | | |
| Palivizumab | RSV-F | Respiratory Syncytial Virus |
| (SYNAGIS ®) | | |
| Panitumumab | epidermal growth factor | colorectal cancer |
| (VECTIBIX ®) | receptor (EGFR) | |
| Ranibizumab | Vascular endothelial | Autoimmune disorders, e.g., |
| (LUCENTIS ®) | growth factor A (VEGF-A) | Macular degeneration |
| Rituximab | CD20 | B cell malignancies, e.g., B bon- |
| (RITUXAN ®, | | Hodgkin's lymphoma |
| MABTHERA) | | |
| Tocilizumab/Atlizumab | IL-6R | Autoimmune disorders including |
| (ACTEMRA ®, | | rheumatoid arthritis |
| ROACTEMRA ®) | | |
| Tositumomab | CD20 | B cell malignancies, e.g., B non- |
| (BEXXAR ®) | | Hodgkin's lymphoma |
| Trastuzumab | HER-2/neu | breast cancer |
| (HERCEPTIN ®) | | |

In some embodiments, various methods can be used to assess whether an antibody has a therapeutic mechanism involving ADCC. In some embodiments, in vitro or ex vivo ADCC assays can be employed, with effector cells from healthy subjects or from subjects suffering from an ADCC treatable disease. In the latter case, the ADCC activity can be compared between high responders, e.g., genotype group I, H/H¹³¹ FcγRIIA and V/V¹⁵⁸ FcγRIIIA, and low responders, e.g., genotype group IX, R/R¹³¹ for FcγRIIA and F/F¹⁵⁸ for FcγRIIIA, as discussed below, where a significant difference in ADDC activity would implicate an ADCC based therapeutic mechanism. Alternatively, the association or linkage of Fcγ polymorphisms that affect ADCC (e.g., FcγIIA and FcγIIIA polymorphisms) and responsiveness to antibody therapy can also be a basis for ascertaining ADCC activity.

In some embodiments, the responsiveness of a subject having an ADCC-treatable disease to an antibody maintenance therapy can be predicted based upon the efficacy of the antibody in depleting target cells during therapy, particularly over a period of time (weeks to months) during maintenance therapy. For instance, a sample is obtained from the individual, the sample is assayed to measure target cell depletion following antibody therapy, and the responsiveness of the individual to an antibody maintenance therapy is predicted based upon the result of that measurement. In some embodiments, the responsiveness of an individual having an ADCC-treatable disease to an antibody maintenance therapy is predicted based upon the efficacy of the antibody in depleting target cells during induction therapy. The extent of depletion of the target cell population may be determined at any time after induction therapy, but typically is determined 2 weeks or more after induction therapy, e.g., 1 month or more, 2 months or more, or 3 months or more after induction therapy, in some instances 4 or 5 months or more after induction therapy, e.g., 6, 7, 8 or 9 or more months after induction therapy. In some embodiments, the responsiveness of an individual having an ADCC-treatable disease to an antibody maintenance therapy is predicted based upon the efficacy of the antibody in depleting target cells during the maintenance phase of the antibody maintenance therapy, i.e. after one or more courses of maintenance therapy, e.g., after 2, 3, 4, 5, or 6 or more courses of antibody maintenance therapy. The extent of depletion of the target cell population may be determined at any time after the initiation of maintenance therapy, but typically is determined 2 weeks after the initiation of maintenance therapy, e.g., 1 month or more, 2 months or more, or 3 months or more after maintenance therapy, in some instances 4 or 5 months or more after maintenance therapy, e.g., 6, 7, 8 or 9 or more months after maintenance therapy.

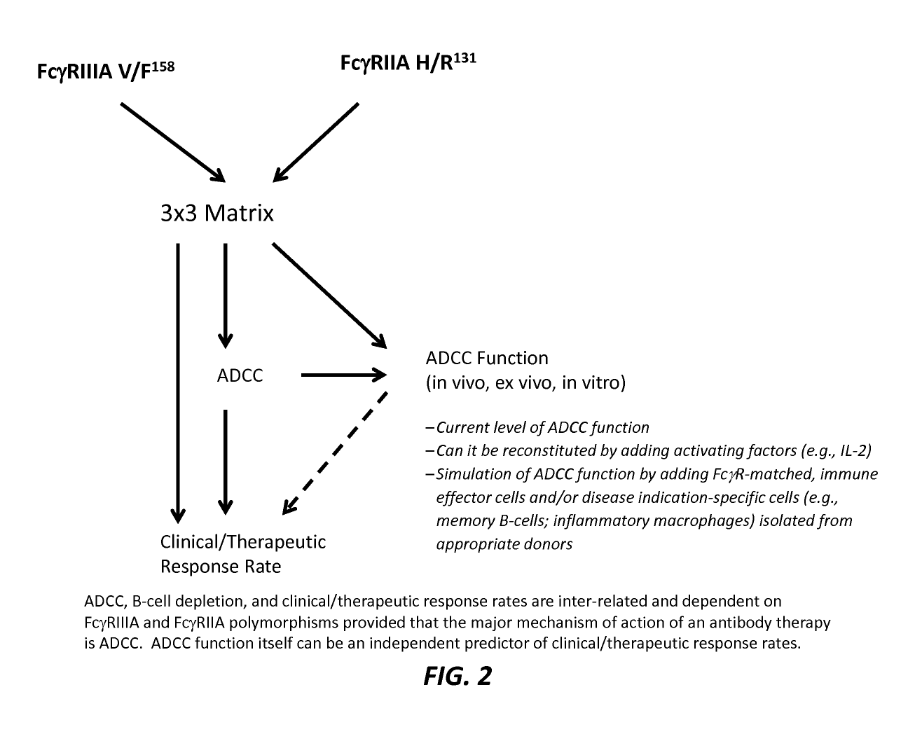

In some embodiments, the responsiveness of an individual having an ADCC-treatable disease to an antibody maintenance therapy is predicted based upon a) the individual's genotype at an FcγRIIA polymorphism and an FcγRIIIA polymorphism and/or b) the efficacy of the antibody in depleting target cells during induction therapy, which is a measure of ADCC function (FIG. 2). In such embodiments, one or more samples is obtained from the individual, a sample is assayed to determine the genotype of the individual from which the sample was obtained with respect to at least one, i.e., one or more, polymorphisms in the FcγRIIA gene and at least one, i.e. one or more polymorphisms in the FcγRIIIA gene, a sample is assayed to measure target cell depletion following antibody induction therapy, and the responsiveness of the individual to an antibody maintenance therapy is predicted based upon the results of either or both of these assessments.

In the various methods of the present disclosure, the subject is evaluated for antibody maintenance therapy or is administered antibody maintenance therapy. Generally, the clinical objective of the administration of antibody maintenance therapy is to (a) achieve complete response and in some cases complete remission, or (b) achieve complete response or remission to maintenance therapy even though the patient had unconfirmed complete response (CRu), or moderate response, or partial response (PR) to induction therapy. The central premise behind this is that upon repeated administration of antibody therapy, when administered at sufficient doses and appropriate defined dosing schedule, can achieve this. The immunological rationale is that when immune effector cells get exposed continuously and repeatedly for longer time periods, even the moderately active immune effector cells which respond to single exposure tend to overcome limitations to achieve excellent and/or complete response.

For example, when healthy individuals representing various ethnicities (Caucasians, African Americans, Caucasians of Norway and Dutch origins; N=1,114) are stratified based on their FcGR-3A V/F¹⁵⁸ and FcGR-2A H/R¹³¹ polymorphisms, the relative distribution (percentage) of individuals in each of the nine genotype groups is fairly constant (see Lehrnbecher, et al. (1999) Blood 94:4220-32; Torkildsen, et al. (2005) Immunology 115:416-21). The percentage of individuals representing the genotype groups-I through IX are: 5.8, 12.2, 8.2, 5.1, 22.6, 19.7, 1.7, 10, 14.8, respectively. This distribution pattern is relatively constant not only in these individual ethnicities but also in other disease settings (see Weng and Levy (2003) J. Clin. Oncol. 21:3940-47). In some disease settings such as lupus, however, a slightly higher percentage of patients are represented in Groups-V, VI, VIII, and IX.

ADCC-dependent antibody therapies use both FcγR-3A and 2A receptors, provided that the Fc region is a human IgG₁ (e.g., rituximab; Weng and Levy (2003) J. Clin. Oncol. 21:3940-47). Particularly, FcγRIIIA V/V¹⁵⁸ and FcγRIIA H/H¹³¹ are the preferred polymorphisms to achieve excellent clinical response in a disease indication such as follicular lymphoma. On the other hand, ADCC-dependent antibody therapies use primarily 2A receptor (and not FcγRIIIA receptor) if the Fc region is a human IgG₂ (e.g., panitumumab; Schneider-Merck, et al. (2010) J. Immunol. 184:512-520). FcγRIIA H/H¹³¹ is the preferred polymorphism. In yet another instance, if an antibody therapy uses a murine IgG₃ Fc framework, FcγRIIA is the preferred receptor, and in fact preferentially uses R/R¹³¹ polymorphism (Cheung, et al. (2006) J. Clin. Oncol. 24:2885-90).

When rituximab, which is a chimeric antibody with a human IgG₁ Fc region, is administered as a single course (e.g., four, weekly infusions) monotherapy to treat follicular lymphoma (B-NHL) patients, Group-I patients were “excellent” responders (˜4%). Patients in a combined group (i.e., pooled Groups-II, III, IV, and VII) were “moderate” responders (˜31%). Patients in another combined group (i.e., pooled Groups-V, VI, VIII, and IX) were classified as “poor” responders (see Weng and Levy (2003) J. Clin. Oncol. 21:3940-47).

In a related single course rituximab monotherapy study (four, weekly infusions of 375 mg/m² per infusion; see McLaughlin, et al. (1998) J. Clin. Oncol. 16:2885-33), for patients who had detailed pharmacokinetic monitoring, the mean serum half-life after the first infusion was 76.3 hours (range, 31.5-152.6), while after the fourth infusion, it was 205.8 hours (range, 83.9-407.0). The maximum observed concentration was higher after the fourth than after the first infusion (mean, 464.7 vs. 205.6 μg/mL, respectively), the clearance was slower (0.0092 vs. 0.0382 L/h), and the area under the curve was greater (86,125 vs. 16,320 μg·h/mL). A significant correlation was found between the number of circulating B-cells at baseline and rapidity of antibody clearance after the first infusion (P=0.01). Thus, repeated weekly infusions leads to (a) significantly higher serum half-life of antibody, (b) significantly higher maximum observed antibody concentration, (c) significantly slower antibody clearance, (d) significantly higher area under the curve for antibody therapy, and (e) better ultimate B-cell depletion.

Given that ADCC is the major mechanism of action by which rituximab exerts therapeutic response, if the immune effector cells are continually exposed to rituximab over several weeks, and preferably months, by way of maintenance therapy which is comprised of induction and maintenance phases, administered either at specified time intervals (syn: re-treatment) or on an as-needed basis, the moderate responders in the single course setting (genotype groups: II, III, IV, and VII per the FcγR polymorphisms) are expected to achieve complete remission or excellent response in maintenance therapy setting, particularly when the maintenance therapy is systematically sustained by taking into account the serum half-life and serum clearance factors into consideration. Similarly, patient groups such as V, and possibly VI and VIII may achieve significantly better response to maintenance therapy compared to a single infusion or single course (four, weekly) therapy. Thus, maintenance therapy, especially when continued over a period of time, can achieve complete remission or complete response in a subset of patients (e.g., rituximab in indications such as follicular lymphoma, CLL, rheumatoid arthritis, SLE, lupus nephritis, multiple sclerosis, systemic vasculitides, etc.), and these patients can be identified a priori by way of determination of their FcγR polymorphisms, or by the determination of ADCC function, or both combined. If the ADCC is the major mechanism of action, notwithstanding the disease indication-specific variables, maintenance therapy can potentially achieve clinical remission and complete response only in specified subsets of patients, as can be determined by the FcγR polymorphisms or ADCC function assay, or both together. That is, despite maintenance therapy, the additional subsets of patients which can also be identified by these features, may not achieve clinical remission and complete response although a better response is feasible. For instance, rituximab maintenance therapy can achieve ˜35-40% complete response in follicular lymphoma (see Hainsworth, et al. (2005) J. Clin. Oncol. 23:1088-95; Hochster, et al. (2009) J. Clin. Oncol. 27:1607-14). Re-treatment therapy in RA leads to ˜15-20% ACR70 values and DAS remission, and ˜35% ACR50 values (see Mease, et al. (2010) J. Rheumatol. 37:917-27; Emery, et al. (2010) Ann. Rheum. Dis. 69:1629-35; Rubbert-Roth, et al. (2010) Rheumatol. 49:1683-93). Based on the FcγR polymorphisms, the combined percentage of individuals belonging to genotype groups-I-IV, VII in the 3×3 matrix is ˜35%.

Thus, in some embodiments, an ADCC-dependent antibody maintenance therapy, e.g., rituximab, could lead to better (a) ADCC (effector) function, (b) overall B-cell depletion or subsets of B-cell depletion, or inflammatory macrophages, e.g., which are effected by function, (c) clinical effectiveness, and (d) therapeutic response. Thus, in certain embodiments, (a) considerably higher percentages of patients can achieve high responsiveness, e.g., complete remission, (b) yet additional identifiable subsets of patients can achieve significantly better complete response (e.g., ACR50 values in RA), and (c) yet additional subsets of patients can achieve significantly better partial response (e.g., ACR20 values in RA). In yet another embodiment, it may be possible to predict the excellent, complete, and moderate responders to ADCC-dependent therapy a priori. In yet other embodiments, it may be possible to predict the poorest (e.g., non-) responders to ADCC-dependent therapy a priori, and hence these patients can be put into alternative treatment regimen. Determination of FcγR-3A, 2A polymorphisms is one such measurement. Alternatively, determination of in vivo, ex vivo, or in vitro ADCC function is yet another measurement.

In some embodiments, maintenance therapy may follow any sort of induction therapy, for example an antibody-based induction therapy, e.g., one of the antibody therapies listed in Table 3; a chemotherapy-based induction therapy, e.g., a cocktail of chemotherapeutics such as cyclophosphamide, doxorubicin, vincristine, and prednisone (CHOP); a combination induction therapy, e.g., an antibody therapy plus a chemotherapy; etc.; induction therapy delivered as a single cycle, induction therapy delivered in more than one cycle, e.g., 2, 3, 4 or more cycles; etc.; induction therapy delivered at a single dose, induction therapy delivered in escalating doses, etc. During the maintenance phase of the maintenance therapy, the antibody may be administered regularly, e.g., a 1 day course of therapy that is administered at 2 or 3 month intervals, a 4 week course of therapy (e.g., 375 mg/m2 once per week) administered at 6-month intervals, etc. In some embodiments, maintenance therapy may be given at fixed and often periodic time points, irrespective of patient's treatment response, e.g., daily, weekly, monthly, and in some instances once every 6, 8, 12, 16, 20, or 24 weeks or more. Alternatively, maintenance therapy may be administered on an “as needed” basis, following a “watch and wait” strategy, e.g., when a patient begins to show symptoms of recurrence of the disease.

For example, using the theragnostic (i.e., procedures guiding the treatment protocols) method described herein, a treatment regimen that comprises regular cycles of antibody maintenance therapy may be prescribed. Alternatively, a “watch and wait” regimen may be prescribed, in which an antibody maintenance therapy is administered as needed to an individual having an ADCC-treatable disease, e.g., following the evaluation of repopulation of the target cell population after depletion with the antibody of interest, e.g., after completion of induction therapy, after the start of maintenance therapy, etc. The extent of repopulation of the target cell population may be determined at any time after the completion of induction therapy or initiation of maintenance therapy, but typically is determined 2 weeks or more after therapy, e.g., 1 month or more, 2 months or more, or 3 months or more after therapy, in some instances 4 or 5 months or more after therapy, e.g., 6, 7, 8 or 9 or more months after therapy. In one instance, faster repopulation of a subset of target cells is a measure of imminent disease relapse, and therefore, calls for an immediate administration of the next dose of antibody therapy.

In the treatment of ADCC treatable diseases (e.g., neoplastic and hyperproliferative diseases, follicular lymphoma, CLL, rheumatoid arthritis, and several autoimmune and inflammatory disorders), the subject is treated with a single course of therapy such as an antibody therapy, preferably antibody monotherapy (e.g., rituximab). For example, four, weekly infusions at 375 mg/m2 of rituximab is one such example of single course therapy. In some instances, one infusion or two infusions or multiple infusions are administered before a patient is subject to evaluation. A key feature of single course therapy is, irrespective of the number of infusions given within a short time frame, e.g., preferably within about two to six weeks, no other therapy is administered for some time afterwards. Some patients may achieve complete remission to single course therapy, and therefore, these subsets of patients may not need concomitant administration of chemotherapies as in hematological malignancies, or immunosuppressive drugs as in the case of autoimmune indications. A majority of the patients may achieve moderate or partial response, while a significant majority of the patients are poor responders. See, e.g., Weng and Levy (2003) J. Clin. Oncol. 21:3940-3947. The disease has a much higher likelihood of relapse in partial and poor responders, unless additional therapeutic interventions are provided subsequent to such single course therapy. Accordingly, administration of a therapy or combination therapies during or following the evaluation phase is considered maintenance therapy, and in such a setting, the said single course therapy is typically referred to as induction therapy.

Generally, the first phase in maintenance therapy is the induction phase. The purpose of induction is to achieve complete response, unconfirmed complete response, or partial response, and in some instances, complete or partial remission. Thus the responders can be identified from absolute non-responders who may not benefit from this therapy such that subsequent administration of this therapy may not be of any therapeutic value in these patients. And in some cases, the objective is to bring down the disease load quickly, preferably by administering chemotherapy such as CHOP or CVP therapies, or a combination therapy consisting of chemotherapy and antibody therapy. Once stabilized, the patient is administered a therapy to maintain the clinical response and such therapy can be an antibody monotherapy, chemotherapy, radiation therapy, etc. Generally induction therapy is administered within short time intervals, preferably within 4-6 weeks.

Chemotherapy in the embodiments herein can comprises cytoxic drugs. Chemotherapeutic drugs can include antimetabolites, e.g., used treatment of autoimmune conditions, and include compounds such as methotrexate, azathioprine, primethamine, and the like. Chemotherapeutic agents also include immune suppressant or immunosuppressive drugs, such as hydroxychloroquine, mycophenolate mofetil, prednisolone, and the like.

The length and dosing cycles of induction treatment can vary depending on the indications. In some instances a single infusion of therapeutically sufficient dose itself is considered an induction therapy. In some other instances, an induction therapy consists of four weekly infusions of the therapy. In B-NHL (follicular lymphoma), for instance, it can be four weekly infusions of rituximab at 375 mg/m2, or eight weekly infusions of rituximab at 375 mg/m2. See, e.g., Hainsworth, et al. (2005) J. Clin. Oncol. 23:1088-1095; and Ghielmini, et al. (2005) Annal. Oncol. 16:1675-1682. In some other instances it can be a combination of rituximab and a chemotherapy involving several cycles of treatment. See, e.g., Hochster, et al. (2009) J. Clin. Oncol. 27:1607-1614; and Ghesquieres, et al. (2012) Blood 120:2650-2657.

Induction phase is followed by an evaluation phase, and typically and ideally for a period of weeks after the administration of the induction therapy. The evaluation phase can be about two, four, six, or more weeks, e.g., for certain oncology indications such as B-NHL and particularly follicular lymphoma it will typically and ideally be between about two and four months, and in autoimmune diseases such as rheumatoid arthritis or SLE, it can be between about six and twelve months. Clinical or therapeutic response should generally be observed during or at the end of the evaluation phase, and such response can be: in cancers, a reduction in tumor load, tumor size, or the like; and in autoimmune disorders, e.g., rheumatoid arthritis, complete or partial reductions of inflammatory symptoms, normal joint function and mobility, or substantial reduction in joint pain. In some instances, there may not be a discrete evaluation phase where patients are evaluated following the induction phase, and they are immediately advanced into maintenance phase.

A next, e.g., typically third, phase is referred to as a maintenance phase, and the objective is to (a) maintain the remission or complete response achieved by induction therapy, or (b) achieve complete response or remission to maintenance therapy even though the patient had unconfirmed complete response (CRu), or moderate response, or partial response (PR) to induction therapy. Maintenance therapy may be given at fixed and often periodic time points, irrespective of patient's treatment response, e.g., daily, weekly, monthly, and in some instances once every 6, 8, 12, 16, 20, or 24 weeks or more. In some other instances, however, a patient may be treated on an as-needed basis. For example, a patient is given the first maintenance dose and put on a watch-and-wait regimen wherein the patient is not treated until particular symptoms appear and at which point the patient is re-treated. The patient may be treated once, twice, thrice, or multiple times (e.g., 5, 7, 9, 11, or more successive courses or cycles), and may be treated in this manner over a period of time, e.g., for two years to four years or more, or such re-treatment regimen is practiced as long as the patient responds to the treatment strategy. Such treatment responses include complete response, excellent response, complete remission, or near complete remission. Thus, in certain embodiments, such re-treatment procedure is considered maintenance therapy.

In some instances, subsequent antibody exposure(s) after the induction therapy can also constitute maintenance therapy, and such exposures may be given weekly or monthly, or once in every two, three, four, or six months or more. In other instances, there is no clear distinction between induction, evaluation, and maintenance phases, and instead all these three phases are merged. For instance, an antibody monotherapy can be given repeatedly at pre-determined time intervals. Alternatively, the induction therapy itself may or may not include antibody therapy. The objective of induction therapy is to enable patients to maintain their health in a disease-free or limited-disease state. In certain clinical contexts, an intermediate consolidation step is considered standard, which is one form of maintenance therapy as used herein. In the oncology setting, the maintenance therapy is generally given to lower the risk of cancer relapse after the first treatment, or for patients with an advanced cancer to prevent the cancer from growing and spreading further. Induction therapy can be in the form of a monotherapy such as antibody therapy, chemotherapy, radiation therapy, or a combination therapy (for example, combination of chemotherapy and antibody therapy, combination of immunotherapy and antibody therapy).

Compared to induction therapy, where the disease load or primary causative agent is typically more dramatic, maintenance therapy will often be directed towards a stage in therapy directed to a lessened load of the primary disease burden. The maintenance therapy may include the therapeutic agent at a lower dose, often more frequent and/or in smaller doses, and typically may provide a different set of negative side effects compared to that of the induction therapy. The physiological impact from lowered primary causative agent, e.g., tumor burden, will typically be less if the induction stage has reduced its targeted cells. Thus, the disease load normally is less dramatic, but the progression of the condition may lead to different or additional clinical issues. In particular, progression of disease may itself result in diminution of ADCC function, e.g., due to depletion of necessary effector cells or pathways, immune-compromised states, higher incidence of bacterial, viral, fungal infections, etc. In the context of either more chronic or episodic type conditions, e.g., various autoimmune conditions, the response to maintenance therapy may be evaluated differently from induction therapy response measures. As such, clinical trials for maintenance therapies may be evaluated differently from induction therapies using the same drug product, e.g., a specific (often monoclonal) antibody therapy. The particular doses in maintenance administrations may vary according to some schemes, e.g., decreasing doses with time, increasing doses with time, or may vary depending upon some measure of need, e.g., evaluation of the health status of the patient.

For example, a patient is given the first maintenance dose and put on a watch-and-wait regimen wherein the patient is not treated until particular clinical symptoms appear and at which point the patient is re-treated. The patient may be treated once, twice, thrice, or multiple times, and may be treated in this manner over a course of time, e.g., for two years to four years or more, or such re-treatment regimen is practiced as long as the patient responds to the treatment strategy. Such treatment responses include complete response, excellent response, complete remission, or near complete remission.

The present disclosure further provides means to predict outcome from antibody maintenance therapy. In various embodiments, the antibody maintenance therapy is administered for at least about 2 months; the antibody maintenance therapy is administered at set intervals of at least about 2 weeks; the antibody maintenance therapy maintains the concentration of the antibody within a factor of about 40 fold over the month after antibody maintenance dosing; the antibody maintenance therapy avoids administering a general cytotoxic drug within about 2, 3, or 4 weeks of antibody maintenance dosing; the antibody maintenance therapy avoids administering an immunosuppressant or antimetabolite drug within about two weeks of antibody maintenance dosing; the stratification reference comprises treatment response from at least 300-500 test subjects; each category of treatment response comprises at least 3-50 test subjects; the stratifying reference comprises between three and seven categories of treatment response, where each category corresponding to a single or more than one genotype group; the stratifying reference has nine categories of treatment response, where each category corresponds to a single genotype group; the predicting of treatment response provides medical decision support; the predicting of treatment response provides medical decision support not to treat with the antibody maintenance therapy; the test subject is treated with the antibody maintenance therapy; or the prediction is combined with a functional ADCC assay result from the test subject to provide medical decision support.

The invention further provides a method for improving response of a patient to antibody therapy, where the patient having an ADCC-treatable disease or condition, the method comprising treating the patient with sufficient repeated antibody maintenance therapy to induce the response of patient to improve from a lesser response to a greater response. In particular embodiments, the response improves from excellent response to disease remission; the response improves from complete response to excellent response; the response improves from partial response to complete response; the ADCC-treatable disease is a neoplastic disease or autoimmune disorder; the ADCC-treatable disease is a B-cell lymphoma, B-cell leukemia, chronic lymphocytic leukemia, systemic lupus erythematosus, lupus nephritis, rheumatoid arthritis, or multiple sclerosis; the patient is treated with antibody maintenance therapy for at least about 3 months; the patient is treated with at least 2 courses of maintenance therapy; the patient is treated with antibody maintenance monotherapy for at least 2 courses; the antibody maintenance therapy maintains the concentration of said antibody within about a 40 fold range in the 4 weeks after antibody dosing; the patient is not administered a general cytotoxic drug within two weeks of antibody dosing; the patient is not administered an immunosuppressant or antimetabolite drug within three weeks of antibody dosing; the patient is a human in FcγRIIIA and FcγRIIA genotype group I, II, III, IV, or VII of Table 1 described herein; the patient is a human in FcγRIIIA and FcγRIIA genotype group V or IX; or the antibody maintenance therapy is administered at intervals of between 1 and 4 months.

Another method provided herein is a method for predicting treatment response to an antibody maintenance therapy for a test subject having an ADCC-treatable disease or disorder, the method comprising determining ADCC function in the subject; and predicting the response to the antibody maintenance therapy, wherein a population of similar test subjects have been stratified for response to the antibody maintenance therapy depending upon the ADCC function. In particular embodiments, the determining occurs when no chemotherapy, immune suppressant, or antimetabolite drug has been administered within about the last 2, 4, or 6 weeks; the prediction also includes a factor of FcγRIIIA and/or FcγRIIA genotype; the determining is performed before any induction therapy and is used to support a medical decision of how to treat said subject; the ADCC-treatable disease is a neoplastic disease or autoimmune disorder; the ADCC-treatable disease is a B-cell lymphoma, B-cell leukemia, chronic lymphocytic leukemia, systemic lupus erythematosus, lupus nephritis, rheumatoid arthritis, or multiple sclerosis; the antibody maintenance therapy is continued for at least about 2 months; the antibody maintenance therapy comprises at least 2 courses of antibody maintenance therapy; the antibody maintenance therapy is administered at set intervals of between about 2-16 weeks; the antibody maintenance therapy is administered as needed at about 2-24 week intervals; the antibody maintenance therapy maintains the concentration of the antibody within about a 40 fold range for 4 weeks following antibody dosing; the antibody maintenance therapy avoids administering a general cytotoxic drug within about 2 or 4 weeks of antibody dosing; the antibody maintenance therapy avoids administering an immunosuppressant or antimetabolite drug within about two weeks of antibody dosing; the determining occurs when no chemotherapy, immune suppressant, or antimetabolite drug has been administered within about the last 1, 2, 3, or 4 weeks; the determining is with an in vivo, ex vivo, or in vitro assay of ADCC function; the determining ADCC function is evaluation of numbers of naïve B-cells, memory B-cells, plasma cell precursors, macrophages, tumor infiltrating macrophages, or NK cells; the determining ADCC function is evaluation of a blood or tissue sample; the predicting treatment response provides medical decision support; the predicting treatment response provides medical decision support not to treat with said antibody maintenance therapy; the subject is treated with the antibody maintenance therapy; or the prediction is combined with a determination of FcγRIIIA and/or FcγRIIA genotype result from the subject to provide medical decision support.

The invention further provides a method of selecting patients having a neoplastic disease or autoimmune disorder for an antibody maintenance therapy, the method comprising evaluating ADCC function in indication-specific patients, and selecting said patients who exhibit the appropriate ADCC function for said antibody maintenance therapy. Preferred embodiments include those where the patients are human and also stratified by responsiveness to antibody maintenance therapy according to FcγRIIIA V/F¹⁵⁸ and FcγRIIA H/R¹³¹ polymorphisms; the evaluating is before any treatment for said disease or disorder; the patients with appropriate ADCC function are those with the top 30% of function; the patients with appropriate ADCC function are those with the top 60% of function; the selected patients are treated with said antibody maintenance therapy; the antibody maintenance therapy avoids treatment with a general cytotoxic drug within about two weeks of antibody dosing; the antibody maintenance therapy avoids treatment with an immunosuppressant or antimetabolite within about 1, 2, 3, or 4 weeks of antibody dosing; the antibody maintenance therapy is continued for at least about 2 months; the evaluation is by an in vivo, ex vivo, or in vitro assay of ADCC function; the evaluating ADCC function is determining the number of naive B-cells, memory B-cells, plasma cell precursors, macrophages, tumor infiltrating macrophages, or NK cells; the evaluating is by assay of a blood or tissue sample; the selected patients are also selected for payment for said treatment; or the selected patients are specifically included as a defined subgroup within a clinical study.

In practicing methods, a subject or patient sample, e.g., cells or collections thereof, e.g., a blood sample or tissue sample, is evaluated to predict responsiveness of the patient to an antibody maintenance therapy. For example, a patient with an ADCC-treatable disease who is responsive to antibody maintenance therapy will experience at least a slowing in disease progression; in some instances, at least a cessation of disease progression; in some instances, an improvement in health, i.e. a reversal of disease progression, a loss of disease symptoms, etc. In contrast, a patient with an ADCC-treatable disease that is not responsive to antibody maintenance therapy will not experience at least a slowing in disease progression, or at least a cessation in disease progression, or an improvement in health. In some embodiments in which the induction therapy comprises antibody therapy, responsiveness to an antibody maintenance therapy is responsiveness to maintenance therapy with the same antibody used in the induction therapy. In some embodiments in which the induction therapy comprises antibody therapy, responsiveness to an antibody maintenance therapy is responsiveness to maintenance therapy with an antibody other than that used in the induction therapy.

It is to be understood that the evaluations for responsiveness will depend on the specific disorder and the standards and methods applied for that disorder. For example, diagnosis and evaluation of cancer treatment are described in, among others, Cancer: Principles and Practice of Oncology, 9th Ed., DeVita, et al. eds., Lippincott Williams & Wilkins (2011); Cohen, et al., Infectious Diseases. 3d ed., (2010); and Merck Manual of Diagnosis and Therapy, Merck Publishing (2011). Diagnosis and evaluation of autoimmune diseases are described in, among others, Autoimmune Diseases: Symptoms, Diagnosis and Treatment, Brenner ed., Nova Science Pub (2011). Diagnosis and evaluation of infectious diseases are described, in among others, Mandell, Principles and Practice of Infectious Diseases: Expert Consult Premium Edition (7th ed.) Churchill Livingstone (2009); and Clinical Infectious Disease, Schlossberg ed., Cambridge Univ. Pr. (2008). Diagnosis and evaluation of allograft rejection are described in, among others, Acute Rejection: Risk Factors, Management and Complications, Kobayashi and Arai eds., Nova Science Publishers (2012); and Transplant Rejection, Russell and Cohn eds., Book on Demand Ltd. (2012).

In some embodiments, such as neoplasms, following obtainment of the genotype from the sample being assayed, the genotype is evaluated to determine whether the subject/host/patient is responsive to the anti-neoplastic therapy of interest. In some embodiments, the obtained genotype may be compared with a reference or control to make a diagnosis regarding the therapy responsive phenotype of the cell or tissue, and therefore host, from which the sample was obtained/derived. The terms “reference” and “control” as used herein mean a standardized genotype to be used to interpret the genotype of a given patient and assign a prognostic class thereto. The reference or control may be a genotype that is obtained from a cell/tissue known to have the desired phenotype, e.g., responsive phenotype, and therefore may be a positive reference or control genotype. In addition, the reference/control genotype may be from a cell/tissue known to not have the desired phenotype, and therefore be a negative reference/control genotype.

In the embodiments herein, any convenient metric may be used to measure and convey predictions of responsiveness to maintenance therapy. For example for oncology indications, responsiveness and associated predictions may be made in terms of remission, progression free survival (PFS), overall survival (OS), relapse-free survival (RFS), time to progression (TTP), and/or event-free survival (EFS) as defined herein and as practiced in the art. Evaluation of target lesions include Complete Response (CR), which is disappearance of all target lesions; Partial Response (PR), which is at least a 30% decrease in the sum of the Longest Diameter (LD) of target lesions, taking as reference the baseline sum LD; Stable Disease (SD), which is neither sufficient shrinkage to qualify for PR nor sufficient increase to qualify for PD, taking as reference the smallest sum LD since the treatment started; or Progressive Disease (PD), which is at least a 20% increase in the sum of the LD of target lesions, taking as reference the smallest sum LD recorded since the treatment started or the appearance of one or more new lesions

Also in oncology indications, evaluation of target lesions include Complete Response (CR), which is disappearance of all target lesions; Partial Response (PR), which is at least a 30% decrease in the sum of the Longest Diameter (LD) of target lesions, taking as reference the baseline sum LD; Stable Disease (SD), which is neither sufficient shrinkage to qualify for PR nor sufficient increase to qualify for PD, taking as reference the smallest sum LD since the treatment started; or Progressive Disease (PD), which is at least a 20% increase in the sum of the LD of target lesions, taking as reference the smallest sum LD recorded since the treatment started or the appearance of one or more new lesions

A specific example is demonstrated by definition of end points for follicular lymphoma. Complete response (CR) required the resolution of all symptoms and signs of lymphoma including bone marrow clearing, for at least 28 days. Partial response (PR) required a ≧50% decrease in the sum of the products of perpendicular measurements of lesions, without any evidence of progressive disease for at least 28 days. Patients who did not achieve a CR or PR were considered non-responders (NR), even if there was a net decrease (<50%) of measurable disease. Time to progression was measured from the first infusion until progression. See, e.g., McLaughlin, et al. (1998) J. Clin. Oncol. 16:2885-33; Cheson, et al. (1999) J. Clin. Oncol. 17:1244-53; and Weng and Levy (2003) J. Clin. Oncol. 21:3940-47.

In the area of autoimmune disorders, different measures of responsiveness or lack thereof to treatment exist among the different autoimmune diseases. Since most are progressive and chronic, they may have similar staging and episodic conditions as found in many oncology conditions. See the American College of Rheumatology (ACR) website www.rheumatology.org. ACR scores represent the percentage of reduction (20%, 50%, 70%) in tender and swollen joint counts, in addition to a corresponding improvement in three of the following five parameters: acute phase reactant (such as erythrocyte sedimentation rate), Patients Global Assessment of Disease Activity, Physicians Global Assessment of Disease Activity, Pain scale, and Health Assessment Questionnaire (HAQ). DAS28 is a measure of disease activity in RA. The score is calculated by a complex mathematical formula, which includes the number of tender and swollen joints (out of a total of 28), the erythrocyte sedimentation rate (a marker of systemic inflammation), and the patient's ‘global assessment of global health’ (indicated by marking a 10 cm line between ‘very good’ and ‘very bad’). A DAS28 score greater than 5.1 indicates severe active disease, less than 3.2 suggests low disease activity, and less than 2.6 is considered DAS remission.

In lupus (SLE), there are two major scoring systems to evaluate the activity of lupus in clinical studies: SLE Disease Activity Index (SLEDAI) and British Isles Lupus Activity Group (BILAG). SLEDAI is a list of 24 items, 16 of which are clinical items such as seizure, psychosis, organic brain syndrome, arthritis, blood vessel inflammation, etc. The other criteria are laboratory results such as urinalysis testing, blood complement levels, increased anti-DNA antibody levels, low platelets, and low white blood cell count. These items are scored based on whether these manifestations are present or absent in the previous 10 days. Organ involvement is weighted. An improvement of this, the SELENA-SLEDAI adds some clarity to some of the definitions of activity in the individual items, but does not change the basic scoring system. BILAG is an organ-specific 86-question assessment based on the principle of the doctor's intent to treat, which requires an assessment of improved (1), the same (2), worse (3), or new (4) over the last month. For screening, treatment, and management of lupus and lupus nephritis, ACR guidelines are adopted (ACR Ad Hoc Committee on SLE Guidelines (1999) Arth. Rheum 42:1785-96; Hahn, et al. (2012) Arth. Care & Res. 64:797-808).

Similar scoring methods have been devised for other immune disorders, e.g., in Sjogren's Syndrome, polymyalgia rheumatica, osteoarthritis, multiple sclerosis, systemic vasculitides, Wegener's granulomatosis, and others. See, e.g., Beers (ed.) The Merck Index of Medical Information; American Autoimmune Related Diseases, www.rheumatology.org, and websites of particular diseases or conditions.

In the embodiments herein, the Fcγ receptor polymorphism can be determined by various methods known in the art. Generally, a sample is obtained from an individual with an ADCC-treatable disease, the sample is assayed to determine the genotype of the individual from which the sample was obtained with respect to at least one, i.e., one or more, polymorphisms in the FcγRIIA gene and/or at least one, i.e. one or more polymorphisms in the FcγRIIIA gene. Any convenient protocol for assaying a sample for the above one or more target polymorphisms may be employed in the subject methods. In some embodiments, the target polymorphism will be detected at the protein level, e.g., by assaying for a polymorphic protein. In some embodiments, the target polymorphism can be detected at the nucleic acid level, e.g., by assaying for the presence of nucleic acid polymorphism, e.g., an single nucleotide polymorphism (SNP) that cause expression of the polymorphic protein.

In some embodiments, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein. Detection of a target polymorphism in a polynucleotide sample derived from an individual can be accomplished by means well known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene; the presence of a non-wild-type splicing pattern of the corresponding mRNA; an alteration in the expression level of the corresponding polypeptide; and/or an alteration in corresponding polypeptide activity. Detailed description of these techniques can be found in a variety of publications, including, e.g., “Laboratory Methods for the Detection of Mutations and Polymorphisms in DNA” G. R. Taylor, ed., CRC Press, and references cited therein (1997).

In some embodiments, genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. See, e.g., “PCR Protocols” in Methods in Molecular Biology, J. M. S. Bartlett and D. Stirling, eds, Humana Press (2000); and “PCR Applications: Protocols for Functional Genomics” Innis et al, eds., Academic Press (1999). Once the region comprising a target polymorphism has been amplified, the target polymorphism can be detected in the PCR product by nucleotide sequencing, by SSCP analysis, or any other methods known in the art. PCR may also be used to determine whether a polymorphism is present by using a primer that is specific for the polymorphism. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163-66; and Saiki et al (1989) Proc. Natl. Acad. Sci. USA 86:6230-34. Exemplary methods for determining FcγIIA and FcγIIIA polymorphisms are described in Delgado et al., 2010, Cancer Res. 70(23):9554-61.

In some embodiments, oligonucleotide ligation can be used to detect polymorphisms. See, e.g., Riley et al. (1990) Nucleic Acids Res. 18:2887-2890; and Delahunty et al. (1996) Am. J. Hum. Genet. 58:1239-1246. In some embodiments, hybridization with the variant sequence may also be used to determine the presence of a target polymorphism. Hybridization analysis can be carried out in a number of different ways. including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Identification of a polymorphism in a nucleic acid sample can be performed by hybridizing a sample and control nucleic acids to high density arrays containing hundreds or thousands of oligonucleotide probes. See, e.g., Cronin et al. (1996) Human Mutation 7:244-255; and Kozal et al. (1996) Nature Med. 2:753-759.

In some embodiments, the genotype is determined by assaying the polymorphic protein. Detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g., fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc. The presence and/or the level of a polymorphic polypeptide may also be detected and/or quantitated in any convenient assay format. In some embodiments, fluorescence-activated cell sorting (FACS) methods can determine the presence or absence of the different polymorphisms on cells isolated from the blood or other biological samples. See, e.g., for FcγRIIA: Böttcher, et al. (2005) J. Immunol. Meth. 306:128-36; and for FcγRIIA: Boruchov, et al. (2005) J. Clin. Immunol. 115:2914-23.

Additional references describing various protocols for detecting the presence of a target polymorphism include, but are not limited to, those described in: U.S. Pat. Nos. 6,703,228; 6,692,909; 6,670,464; 6,660,476; 6,653,079; 6,632,606; 6,573,049; the disclosures of which are herein incorporated by reference.

As described in the present disclosure, the antibody selected for maintenance therapy functions through an ADCC mechanism. In some embodiments, in addition to use of the genotype of the Fcγ polymorphism to predict responsiveness to antibody maintenance therapy, any of the method of predicting, stratifying, or treatment as described herein can further comprise measuring the level of ADCC function or capacity of the subject of interest.

Measuring or ascertaining the level of ADCC function in the subject provides another independent factor in predicting whether the subject will have a clinically significant (or insignificant) response to antibody maintenance therapy. Absent or low ADCC function in the patient would predict low responsiveness to antibody therapy while a high or robust capability would predict excellent or good responsiveness to antibody therapy. In some embodiments, each genotype group for one or more Fcγ polymorphisms can be further stratified based on ADCC function, thereby providing a more refined stratification for predicting responsiveness, and hence better selection of subjects and treatment with the antibody based therapy. As noted above, in some embodiments, ADCC function without genotype based stratification can be used to determine ADCC function of the subject.

In some embodiments, the subject selected for antibody maintenance therapy has at least 10% or more, 20% or more, 30% or more, 40% or more, 50% or more, 60% or more, 70% or more 80% or more, or 90% or more of ADCC capacity/function as compared to a control subject, wherein the control subject is a healthy subject (e.g., human) or a treatment naïve subject (e.g., human) afflicted with the ADCC treatable disease or disorder. In some embodiments the control is a subject (e.g., human) with the ADCC treatable disease and a genotype for Fcγ polymorphisms that is a excellent or good responder to antibody therapy, e.g., a genotype for a Fcγ receptor polymorphism comprising H/H¹³¹ for FcγRIIA and V/V¹⁵⁸ for FcγRIIIA. ADCC capacity of a subject is the maximum ADCC function that the subject can possibly achieve under optimal physiological conditions, and this is influenced by the FcγRIIA and FcγRIIIA polymorphisms, the type of chemotherapy used immediately prior to the administration of antibody therapy, etc. ADCC capacity also provides a measure of tunable or activatable ADCC function in the subject, and this can be measured ex vivo by adding, for instance, factors such as IL-2 to activate NK cells. ADCC function means the actual level of ADCC the subject exhibits at any given point during the treatment.

The ADCC function or capacity can be determined by various methods available to the skilled artisan, as further discussed below. In some embodiments, a sample obtained from the subject and containing ADCC effector cells, including macrophages, natural killer (NK) cells, neutrophils, eosinophils or combinations thereof, such as a blood sample can be used for in vitro or ex vivo ADCC assays. In some embodiments, the sample can be further processed to prepare isolated cell preparations containing one or more of the effector cells. The sample can be obtained before, during and/or after induction therapy, as well before during and/or after the maintenance phase of maintenance therapy.

In some embodiments, the ADCC function or capacity can be measured by a target cell radiolabel release assay, target cell enzyme release assay, a target cell depletion assay, or target cell repopulation assay. A target cell radiolabel release assay suitable for the present methods can use release of ⁵¹Cr, Europeum (Eu), or ³⁵S, such as described in Nelson et al., Current Protocols In Immunology, Wiley Publishers, 2001, and Patel and Boyd, 1995, J Immunol Methods. 17:184(1):29-38. A target cell enzyme release ADCC assay can be based on measurement of a released cellular enzyme, such as glyceraldehyde-3-phosphate dehydrogenase (GAPDH), available commercially as aCella-TOX®. Other similar assays will be apparent to the skilled artisan.

Determination of ex vivo ADCC in a given patient can be performed by withdrawing freshly harvested blood samples before and after the administration of antibody therapy, preferably antibody maintenance therapy, e.g., rituximab antibody maintenance therapy to treat RA. Blood samples can be withdrawn on a regular basis, e.g., about every 1, 2, 4, 6, 8, 12, 16, etc., weeks, for an appropriate period, which may be, e.g., at least 1, 2, 4, 6, 8, 10, 12 or more months.

In some embodiments, the ADCC function or capacity can be determined by target cell depletion or repopulation assays, which measure levels of specific cells targeted by the antibody therapeutic. For example, maintenance treatment with an anti-CD20 can measure B cell populations, which are targeted by the antibodies, for example in B non-Hodgkins lymphoma or autoimmune disease such as SLE. A person of skill in the can determine the relevant target cells and subsets of B cells based on the antibody and the disease being treated. Samples for measuring target cell levels can be obtained before and after antibody induction therapy and/or before and after antibody maintenance therapy. In some embodiments, the number of target cells is determined 2 weeks or more after induction therapy, e.g., 1, 2, 3, 4 or more, 2 months or more, or 3 months or more after induction therapy, in some instances 4 or 5 months or more after induction therapy, e.g., 6, 7, 88, or 9 or more months after induction therapy. In some embodiments, the number of target cells is determined during antibody maintenance therapy, i.e., after one or more courses of maintenance therapy, e.g., after 2, 3, 4, 5, or 6 or more courses of antibody maintenance therapy. The extent of depletion or repopulation of the target cell population may be determined at any time after the start of maintenance therapy, but typically is determined 2 weeks or more after maintenance therapy, e.g., 1 month or more, 2 months or more, or 3 months or more after maintenance therapy, in some instances 4 or 5 months or more after maintenance therapy, e.g., 6, 7, 8, or 9 or more months after maintenance therapy. Generally, a decrease or depletion of target cell will be indicative of positive ADCC function in a subject. Conversely, a repopulation or increase in target cells will be (a) indicative of decreased ADCC function in a subject, or (b) disease relapse.

In some embodiments, functional ADCC assay involves determination of depletion of specific sets of cells, preferably functional subsets, such as B-cells or macrophages, or subsets of B-cells such as CD19+ naive B-cells, CD27+ memory B-cells, CD38+ preplasma cells, or FRβ+ inflammatory macrophages, etc., and such assay involves highly sensitive methods such as MRD-flow cytometry to detect ultra-low levels (e.g., about 5 CD27+ memory B-cells per ml of blood) of specific cells that are depleted due to ADCC.

In some embodiments, functional ADCC assay involves determination of re-population of specific sets of cells, such as B-cells, specifically subsets of cells B-cells such as CD27⁺ memory B-cells, CD38⁺ preplasma B-cells, or FRβ⁺ inflammatory macrophages, etc., and such assay involves highly sensitive methods such as MRD-flow cytometry to detect ultra-low levels (for example, the number of CD38⁺ preplasma B-cells are 100, 4, 10, 200, 800 per microliter of blood after, e.g., 2, 4, 6, 8, and 12 weeks, respectively, after the administration of antibody therapy; the corresponding pre-treatment cell density, e.g., being about 900 cell per microliter of blood) of specific cells that are both depleted and subsequently re-populated (800 cells per ml at week-12, in this example) during the course of antibody maintenance therapy. In this example, re-population pattern shows that indeed the antibody therapy leads to the initial ADCC-mediated depletion of already existing CD37⁺ B-cells, and while the antibody therapy continues to provide therapeutic effect and continued depletion of B-cells, regenerated CD37+ preplasma B-cells re-populate after an appropriate period of 6 weeks after the initiation of antibody treatment (e.g., see Roll, et al. (2008) Arth. Rheum. 58:1566-75). In one aspect, the functional ADCC assay can be used to monitor depletion patterns of some specific cell types of B-cells, macrophages, T-cells, etc. In a second aspect, the functional ADCC assay can be used to monitor the re-population patterns of specific cell types such as B-cells, macrophages, and T-cells. Thus, in some embodiments, ADCC functional assay(s) can provide real-time, that is, as the patient undergoes maintenance therapy over a period of several months, therapeutically relevant information such as, rates of depletion and re-population of disease-specific cells, and level (complete or partial) of depletion, which can then be correlated to treatment response, non-response, disease remission, or relapse phases. Thus in various embodiments, such ADCC function measurements, for instance, in rheumatoid arthritis or SLE, can predict the time-to-disease remission and the time-to-disease relapse, preferably two to four weeks, or preferably 6-10 weeks before the actual clinical symptoms disappear or re-appear, respectively.

Target cell depletion or repopulation (e.g., proliferation) may be represented by many convenient metrics. For example, target cell depletion or repopulation may be a comparison, i.e., ratio, of target cells in a blood or tissue sample before and after therapy, e.g., the ratio of B cells in a blood sample of an B-NHL patient after induction therapy versus before induction therapy, or the ratio of B cells in a blood sample of an B-NHL patient after the start of maintenance therapy versus before the start of maintenance therapy. Alternatively, target cell depletion or proliferation may be a measurement of the absolute number of target cells in a blood or tissue sample after induction therapy, e.g., the actual number of B cells in a blood sample of an B-NHL patient after induction therapy, or the actual number of B cells in a blood sample of an B-NHL patient after 1 or more courses of maintenance therapy, e.g., after 2, 3, 4, 5, or 6 or more courses of maintenance therapy, etc. As another example, target cell depletion or proliferation may be a measurement of the percentage of target cells in a sample after therapy relative to a larger population of cells in the sample, e.g., the percentage of target B cells in a blood sample of an NHL patient after induction therapy relative to the total number of leukocytes in the sample, or the percentage of target B cells in a blood sample of an NHL patient after 1 or more courses of maintenance therapy relative to the total number of leukocytes in the sample, e.g., after 2, 3, 4, 5, or 6 or more courses of maintenance therapy, etc. For instance, as measured by MRD-FC, the actual numbers of naïve (CD 19⁺⁺, CD27⁻⁻, CD38^{+/-}) and memory (CD19⁺⁺, CD27⁺, CD38^{+/-}) B cells just before the treatment and 2 weeks after the rituximab treatment in a B-NHL subject are: 300, 20; 140, 5 cells per microliter of blood sample, respectively. As another example, target cell depletion or proliferation may be a measurement of the percentage of target cells in a sample after therapy relative to a larger population of cells in the sample, e.g., the percentage of target B cells in a blood sample of an B-NHL patient after induction therapy relative to the total number of leukocytes in the sample, or the percentage of target B cells in a blood sample of an B-NHL patient after 1 or more courses of maintenance therapy relative to the total number of leukocytes in the sample, e.g., after 2, 3, 4, 5, or 6 or more courses of maintenance therapy, etc.

Degree of depletion is a significant predictor of therapeutic response. Upon antibody treatment, a patient can achieve complete or partial depletion as measured by highly sensitive flow cytometry (e.g., MRD-FC) or any such means. Detection of extremely low amounts of B-cells (e.g., 1-100 cells/ml) and of relevant subsets of B-cells is necessary to correlate to therapeutic response. Subsets of B-cells include naive B-cells, memory B-cells, and preplasma cells. Degree of depletion in specific compartments such as peripheral blood (in the case of B-NHL, CLL, rheumatoid arthritis), synovium (in the case of rheumatoid arthritis), lymph nodes and bone marrow. Patients who have difficulty achieving peripheral clearance may be more likely to have difficulty clearing other compartments, when given the same dose of rituximab, thus leading to subsequent poorer response.

Complete depletion (e.g., greater than about 70%, preferably 75, 80, 85, 90, or 95% depletion) correlates to better therapeutic outcomes while partial depletion relates to poorer therapeutic outcomes. This can be measured (a) by administering the antibody to patients, and withdrawing blood samples over several weeks as part of the treatment follow-up, or (b) by the determination of ex vivo ADCC potential of a patient. Rate of depletion is equally a significant predictor of treatment response. Faster rate of depletion (e.g., on the order of days and weeks, e.g., about 3, 5, 7, 10, 13, 17, 21, 25, 30, 35, 40, or more days) correlates to better therapeutic outcomes. Conversely, slower rate of depletion (on the order of several weeks, e.g., about 1, 2, 3, 4, 5, 6, 7 or more) correlates to partial or poor therapeutic outcomes.

Complete depletion at a slower rate can be a predictor of poor treatment response. Faster but incomplete depletion can also be a predictor of poor treatment response. Rate of re-population (e.g., of a target cell) is yet another significant predictor of treatment response. Faster rate of re-population (e.g., on the order of days and weeks, which may be re-population by about 30-50%, or up to about 75% in about 3, 5, 7, 10, 14, 18, 21, 25, 28, 31, or 35 days or more) correlates to partial or poor therapeutic outcomes, while slower rate of re-population correlates to complete or excellent therapeutic outcomes (e.g., on the order of weeks and months).

Both faster rate of depletion and complete depletion are indicators of better therapeutic response. Initial target cell density prior to the initiation of treatment is another indicator of treatment response.

For hematological oncology indications, the functional depletion/repopulation will typically be the depletion of the hyperproliferative cell type, e.g., the decrease in number of hyperproliferative cells of interest. The functional cells may, under certain circumstances, be indirectly affected, and in some cases, may actually increase, if the pathways of response are such that the cell type will be increased in response to the decrease of the hyperproliferative cells. The specific functional subset of cells evaluated might be affected by the hyperproliferative cells.

For general oncology indications, the desired functional depletion/repopulation will typically be of the cancer cells. The tumor burden would decrease, as might secondary cell types which respond to tumorigenic signals. Where the cancer results from aberrant growth or proliferation signaling, cells which are affected, e.g., tumor cells themselves, can be monitored. In some circumstances, the presence of cancer cells may affect numbers of functional cells which would normally function to clear the hyperproliferative cells, and that functional subset may increase upon effective treatment. The cells may be directly or indirectly responding to proliferation or growth signals, and may represent a decrease as intended, or may respond to the decrease in cancer cells by increasing various particular different cell types.

In the autoimmune disease context, cells of functional interest will typically be responsible immune cells, often B cells, inflammatory macrophages, or inflammatory NK cells. In B cell autoimmune processes or Ig producing conditions, positive response may be indicated by decrease in associated B cell subset numbers, or T cell or other immune cell subsets which promote B cell functions. In other circumstances, e.g., in inflammatory conditions, macrophages may be depleted as treatment succeeds.

For microbial infection indications, the functional depletion will generally be the infectious agent, whether cells or viruses, or secondary responders. Cells which may indirectly be affected can be evaluated for decreasing, or increasing, as appropriate.

Likewise, in a rejection response, the cell of interest will typically be an immune cell involved in rejection. In other cases, the cells may respond indirectly to the rejection by increasing when a desired response is intended. Or the desired cell for evaluation may be repopulating after the induction treatment.

Many convenient methods may be used to assess the number of target cells in a sample, e.g., flow cytometry (conventional flow cytometry, minimal residual disease flow cytometry (MRD), etc.), histochemistry, immunohistochemistry, etc. In some embodiments, ex vivo and/or in vivo methods can be used to detect and quantitate the cells. The In some embodiments, detection of the cells by these methods can be based on a specific marker, e.g., CD20 for B cells, present on the target cells. In some embodiments, the antigen that is the target of the induction therapy, e.g., for B cells targeted by rituximab, CD20, is used as a marker. In some embodiments, an antigen that is not the target of the induction therapy, e.g., for B cells targeted by rituximab, CD19, is used as a marker. See, e.g., Shapiro, H. M., Practical Flow Cytometry, Wiley Liss 4^{th} Ed. (2003); and Davis et al., 1999, Clin Cancer Res. 5:611-615.

In some embodiments, assessing ADCC function or capacity may rely on measuring the expression of Fcγ receptors affecting ADCC activity, particularly on ADCC effector cells. As will be apparent to the skilled artisan, low expression of FcγIIIA (V/V¹⁵⁸) receptors as compared to appropriate controls would implicate or predict weak or poor ADCC capacity. Conversely, high expression of FcγIIIA (VV¹⁵⁸) receptors would implicate or predict excellent to good ADCC capacity. The expression of Fcγ can be measured at the mRNA level or at the protein level by methods available to the skilled artisan. In some embodiments, Fcγ is measured, for example, using techniques of hybridization, reverse transcriptase based PCR, nucleic acid arrays, and the like. See, e.g., Current Protocols in Molecular Biology, Ausubel, F M. ed., John Wiley & Sons (1995), updates to 2011. In some embodiments, expression of Fcγ receptors is measured at the protein level, for example by use of antibodies and techniques such as ELISA, immunoblotting, and quantitative fluorescence activate cell sorting. See, e.g., Current Protocols in Immunology, Coligan ed., John Wiley & Sons (2005).

In some embodiments, an additional indicator that can be applied to assess ADCC function or capacity is the level of immune cells, particularly ADCC effector cells, as compared to appropriate control levels, e.g., healthy subject, particularly with the same Fcγ genotype. The level of such cells (e.g., cells per volume of blood) can be determined using methods as described herein and by methods well known in the art, e.g., FACS using antibodies to specific markers on the cells. Specific immune cells serving as useful indicators of ADCC function include, among others, natural killer (NK) cells, monocyte derived macrophages, neutrophils, and eosinophils. In some embodiments the level of target cell and effector cells can be determined concurrently, for example by employing FACS using an antibody specific to the target cell and a second antibody specific to the ADCC effector cell.

In some embodiments, the methods find use in pharmacogenomic applications. In these applications, a subject/host/patient is first diagnosed for the presence or absence of a responsive phenotype using a protocol such as the diagnostic protocol described above. The subject is then treated using a pharmacological protocol, where the suitability of the protocol for a particular subject/patient is determined using the results of the diagnosis step. For example, where the identified phenotype is responsive, e.g., based on an FcγR genotype, or the extent of target cell depletion following induction therapy or following the start of maintenance therapy, an appropriate antibody maintenance therapy treatment protocol is then employed to treat the patient, e.g., maintenance therapy on a regular basis, or on an as-needed basis. Alternatively, where a patient is identified as having a non-responsive phenotype, e.g., based on an FcγR genotype, or the extent of target cell depletion following induction therapy or following the start of maintenance therapy, other antibody therapies, or non-antibody therapeutic interventions, e.g., chemotherapeutic protocols, are then employed.

In some embodiments, the above-obtained information, including antibody ADCC function, is employed to give a refined probability prediction as to whether a subject will or will not respond to a particular therapy. For example, different degrees of target cell depletion can be predictive of responsiveness to antibody maintenance therapy. Complete depletion, e.g., a reduction in target cell number to 1% or less, e.g., 3 cells per microliter or less, after a first cycle of induction therapy is predictive of excellent responsiveness o antibody maintenance, i.e. a 90% chance or more that the patient will be responsive to maintenance therapy. Incomplete depletion after induction, but complete depletion after a first or second cycle of maintenance therapy is predictive of good (80% or more chance) or moderate (60% or more chance) responsiveness to antibody maintenance therapy. Incomplete or partial depletion, on the other hand, is predictive of weak (>40-50% chance) or poor (<25% chance) responsiveness to antibody maintenance therapy.

While the level of target cells (e.g., depletion or repopulation) and or ADCC effector cells can used in combination with Fcγ receptor based stratification, in some embodiments responsiveness to antibody therapy, including antibody maintenance therapy, can be predicted without use of genotype based stratification by measuring the level of target cells and/or ADCC effector cells.

Accordingly in some embodiments, a method for predicting responsiveness of a subject having an ADCC treatable disease or disorder to antibody therapy, including antibody maintenance therapy, can comprise:

measuring depletion or repopulation of a cell population targeted by an antibody induction therapy or an antibody maintenance therapy to obtain a result; and

predicting the responsiveness of the subject to antibody therapy or an antibody maintenance therapy based on the result.

In some embodiments, a method for predicting responsiveness of a subject having an ADCC treatable disease or disorder to antibody therapy, including antibody maintenance therapy, can comprise:

measuring level of an ADCC effector cell to obtain a result; and

predicting the responsiveness based on the result.

In some embodiments, the method of predicting responsiveness to antibody maintenance therapy based on measuring target cells and/or ADCC effector cells can be applied to a method of treating a subject with an ADCC treatable disorder. Accordingly, in some embodiments, the method for treating a subject having an ADCC treatable disease or disorder with antibody maintenance therapy can comprise:

(a) measuring depletion of a cell population targeted by an antibody induction therapy or an antibody maintenance therapy to obtain a result; and

(b) classifying the individual into a responsiveness groups based on the result to obtain a classification; and

(c) administering antibody maintenance therapy to the selected subject. either as a regular regimen or an as needed regimen based on the classification, wherein the regimen is selected based on the individuals classification in a responsive group.

In some embodiments, the methods can be applied to a variety of ADCC treatable disorders, as disclosed herein, including a neoplastic disease, an autoimmune disease, an inflammatory disease, a microbial infection, or allograft rejection. An exemplary disease is B non-Hodgkin's lymphoma (B-NHL), where the targeted cell population is B cells.

In some embodiments, the antibody maintenance therapy is administered regularly. In some embodiments, the antibody maintenance therapy is administered as needed.

In some embodiments, the methods can further comprise genotyping the individual for an FcγRIIA polymorphism and/or an FcγRIIIA polymorphism, wherein predicting responsiveness of the individual to an antibody maintenance therapy is based upon the result of the depletion measurement and the result of the genotyping. In light of the teachings herein, any of the methods described in the present disclosure for using Fcγ receptors polymorphisms in predicting responsiveness to antibody maintenance therapy can be used in combination with the functional ADCC assays.

In view of the teachings of the present disclosure, the ability to predict responsiveness of subjects to antibody maintenance therapy allows a health care provider to assess and select various treatment options, for instance, selecting one therapy from a panel of several other therapeutic options available, that would likely have the most benefit for the patient, and conversely exclude use of treatments that would have insignificant benefit on treatment outcome. Accordingly, a method for selecting a treatment option for an ADCC treatable disease can comprise:

determining a genotype of a human subject for one or more Fcγ polymorphisms affecting ADCC activity, wherein the human subject has an ADCC treatable disease; and

stratifying one or more treatment options based on the determined genotype of the Fcγ polymorphism, wherein the treatment options comprise at least antibody maintenance therapy for the ADCC treatable disease.

In some embodiments, the stratifying of the various treatment options is done by comparing the determined genotype to a reference stratification that relates responsiveness to antibody and/or antibody maintenance therapy to genotypes of the Fcγ polymorphism affecting ADCC activity. As noted herein, the stratification allows predicting the responsiveness to antibody maintenance therapy. Subjects whose responsiveness is excellent or good can be given the appropriate antibody maintenance therapy, while subjects whose responsive is weak or poor can be given alternative therapies, such as chemotherapy or combination therapies that would benefit the patient.

As referenced in the present disclosure, the Fcγ polymorphism affecting ADCC activity can be based on one or more FcγRIIA polymorphisms and/or one or more FcγRIIIA polymorphisms described above, particularly amino acid position 131 of FcγRIIA and amino acid position 158 of FcγRIIIA. Accordingly, in some embodiments, the treatments options can be selected based on the genotype groups presented in Table 1 and the corresponding genotype-responsiveness presented in Table 2, above.

In some embodiments, the treatment option can comprise antibody maintenance therapy for a subject in genotype group (a), (b) or (c) in Table 2, given the likelihood of excellent to good responsiveness. Moreover, a subject in genotype group (a), (b) or (c) can be given induction therapy with an antibody therapeutic, followed by the antibody maintenance therapy.

In some embodiments, the treatment option for a subject in genotype group (d) or (e) can exclude antibody body maintenance therapy as a treatment option. In some embodiments, a subject in genotype group (d) or (e) can be excluded from both induction therapy and maintenance therapy with an antibody. In some embodiments, the treatment options for a subject in genotype group (d) or (e) comprise a chemotherapy with a chemotherapeutic agent. In some embodiments, a treatment option for a subject in genotype group (d) or (e) includes chemotherapy for induction therapy as well as for maintenance therapy.

It is to be understood that the treatment option will depend on the disease or disorder being treated, as described herein, e.g., neoplastic disease, an autoimmune disease, an inflammatory disorder, a microbial infection, or allograft rejection, and that a person of skill in the art can select the appropriate treatment options available to the skilled artisan in view of the guidance and teachings of the present disclosure. See, e.g., Merck Manual of Diagnosis and Therapy, Merck Publishing (2011).

In some embodiments, the selection of a treatment option includes assessment of ADCC function or capacity, as further described herein.

In another aspect, the advantages of selecting patients who are likely to respond to antibody maintenance therapy can also provide advantages in the conduct of clinical trials. For example, drug developer may want to focus a clinical trial on patients expected to respond favorably to an antibody treatment regimen while excluding non-responders and/or poor responders from the dataset. This selection may provide very high response to treatment of the included patient subset, thereby increasing the chances of regulatory approval and providing a more robust, positive clinical outcome. Accordingly, in some embodiments, a method of selecting a subject for inclusion in a clinical trial to evaluate an antibody maintenance therapy for treating an ADCC treatable, comprises:

determining a genotype of a human subject having an ADCC treatable disease for an Fcγ receptor polymorphism affecting ADCC activity;

using the determined genotype of the Fcγ receptor polymorphism in deciding inclusion or exclusion of the subject in the clinical trial for the antibody maintenance therapy.

In some embodiments, the term “inclusion” in the context of a clinical trial refers to including the subject in the clinical trial itself or in some embodiments, selecting the clinical data for analysis, even though the clinical trial has been conducted in an all-corners (unstratified) population. Similarly, the term “exclusion” in the context of clinical trials refers to excluding the subject from the clinical trial itself or excluding the clinical data in the analysis.

In some embodiments, the step for deciding inclusion or exclusion in a clinical trial can comprise comparing the determined genotype to a reference stratification relating responsiveness to one or more antibody treatments to genotypes of the FcγR polymorphism, wherein the antibody treatment is used to treat a second ADCC treatable disease.

It is to be understood that the reference stratification can be based on antibody that is the same or different from the antibody of interest. For example, in some embodiments, the antibody of interest and the antibody used for the reference stratification can be the same. In such embodiments, where the second ADCC treatable disease is the same as the second treatable disease, the reference stratification can be based on a clinical trial or other study examining the ADCC treatable disease in a different treatment context, i.e., different from maintenance therapy, for example, induction therapy. In some embodiments, where the antibody used for the reference stratification is the same as the antibody of interest, the second ADCC treatable disease can be different from the ADCC treatable disease of interest.

In some embodiments, the reference genotype-responsiveness stratification can be obtained from a clinical trial or investigator-sponsored study conducted for an antibody different from the antibody of interest on a second ADCC treatable disease, where the second ADCC treatable disease is the same or different from the ADCC treatable disease of interest.

In some embodiments, the reference genotype-responsive stratification can be compiled from clinical trials or other studies conducted for two or more antibodies, which can be same as or different from the antibody of interest. In some embodiments, the second ADCC treatable disease can be two or more different ADCC treatable diseases.

In the methods of selecting a subject for inclusion in a clinical trial, the Fcγ receptor polymorphism used can be one or more FcγRIIA polymorphisms and/or one or more FcγRIIIA polymorphisms described above, particularly amino acid position 131 of FcγRIIA and amino acid position 158 of FcγRIIIA. In some embodiments, the stratification can be based on the genotypes presented in Table 1. In some embodiments, the reference stratification can comprise the genotype-responsiveness association presented in Table 2 such that inclusion or exclusion of a subject in a clinical trial can be based on the stratification in the table.

Accordingly, in some embodiments, a subject stratifying in genotype group (a), (b) or (c) in Table 2 can be selected for inclusion in a clinical trial for evaluating an antibody maintenance therapy to treat an ADCC treatable disease. In some embodiments, the a subject stratifying in genotype group (b) or (c) can be selected for inclusion in the clinical trial. Conversely, in some embodiments, a subject in genotype group (d) or (e) is excluded from the clinical trial.

In some embodiments, for conducting the trial, the subject is treated with the antibody maintenance therapy following induction therapy, and evaluated for efficacy of the antibody maintenance therapy.

Similar to the other methods described herein, the selection a subject for inclusion in a clinical trial can be an independent assessment of ADCC function or capacity, as further described herein.

Given the advantages of predicting responsiveness to antibody therapy and ability to select treatment options that have a likelihood of having a positive treatment outcome, the methods herein provide an additional benefit in assisting management of healthcare. For example, the methods herein allow a healthcare manager to make certain treatment options in order to achieve better therapeutic outcomes and reduce burden on financial resources.

Accordingly, in some embodiments, the present disclosure provides a healthcare management method for determining a health service payer coverage of antibody maintenance therapy for treating an ADCC treatable disease, the method comprising:

obtaining genotype information of a human subject having an ADCC treatable disease for a Fcγ receptor polymorphism affecting ADCC activity;

determining health service payer coverage of the antibody maintenance therapy based on the genotype information for the Fcγ receptor polymorphism.

In some embodiments, determining health payer coverage can comprise (a) comparing the genotype information to a reference stratification relating responsiveness to one or more antibody maintenance therapies to genotypes of the Fcγ polymorphism, (b) measuring ADCC function or capacity, or (c) using both information, as described throughout the present disclosure.

In some embodiments, the reference stratification can comprise data stored in a computer memory. In some embodiments, the comparing of the genotype information to the reference stratification can be carried in a computer.

In some embodiments, the method further comprises determining a treatment outcome for the antibody maintenance therapy. Thus a treatment outcome that is weak or poor response can be a basis for not covering the maintenance therapy while a treatment outcome that is excellent or good can be a basis for approving coverage of the maintenance therapy.

Similar to the other methods described herein, the Fcγ receptor polymorphism affecting ADCC activity can be based on one or more FcγRIIA polymorphisms and/or one or more FcγRIIIA polymorphisms described above, particularly amino acid position 131 of FcγRIIA and amino acid position 158 of FcγRIIIA. Accordingly, in some embodiments, determining coverage can be selected based on the genotype groups presented in Table 1 and in some embodiments, the corresponding genotype-responsiveness presented in Table 2, above.

In some embodiments, the method further comprises determining a treatment option, as described in the present disclosure.

Accordingly, in some embodiments, a subject stratifying in genotype group (a), (b) or (c) in Table 2 can be approved for antibody maintenance therapy to treat an ADCC treatable disease. In some embodiments, a subject stratifying in genotype group (b) or (c) can be approved for antibody maintenance therapy to treat an ADCC treatable disease. In some embodiments, a subject in genotype group (d) or (e) is can be approved for a different treatment option, such as chemotherapy.

In some embodiments, the determining of coverage, the comparing of the genotype information, treatment outcome, and the treatment options can be reported in electronic, web-based, or paper form to the subject, a health care payer, third party payer, a health care provider, a physician, a pharmacy benefits manager or a government office.

### Reagents Devices and Kits

The present disclosure also relates to reagents, devices and kits thereof for practicing one or more of the above-described methods. For example, kits may comprise one or more elements for genotyping a patient to identify a FcγRIIA polymorphism, and one or more elements for genotyping a patient to identify a FcγRIIIA polymorphism. Such elements may be, for example, oligonucleotides, e.g., for PCR and/or sequencing the FcγRIIA and FcγRIIIA loci, for hybridization of the FcγRIIA and FcγRIIIA loci or FcγRIIA and FcγRIIIA RNA, etc., or as another example, antibodies, e.g., an antibody specific for the H¹³¹ or R¹³¹ allele of FcγRIIA, and an antibody specific for the V¹⁵⁸ or F¹⁵⁸ allele of FcγRIIIA. Additionally, or alternatively, kits may comprise one or more elements for detecting and measuring cells in a human sample, i.e. cells that are targeted for depletion and/or repopulation by an antibody induction therapy used to treat an ADCC-treatable disease. Such elements may include, for example, antibodies, e.g., an antibody that is specific for a marker on the targeted cell, an antibody that is specific for a larger population of cells that comprise the targeted population, etc., a vital dye for determining cell viability, etc. The kit may further comprise a reference that correlates a genotype in the patient and/or the extent of target cell depletion and/or repopulation in a patient with patient groups having known responsiveness to the antibody maintenance therapy.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means may be present in the kits.

The following examples are offered by way of illustration and not by way of limitation.

### EXAMPLES

### Example 1

### B Non-Hodgkin's Lymphoma Maintenance

A patient diagnosed with B non-Hodgkin's lymphoma undergoes induction therapy with R-CHOP, a therapy comprising rituximab and the chemotherapeutic agents such as cyclophosphamide, doxorubicin, vincristine, and prednisone. The induction therapy puts the patient's disease in remission. The patient and the physician must now decide whether to proceed with antibody maintenance therapy using rituximab and if so, under what regimen. The physician has a technician prepare genomic DNA from a sample of peripheral blood mononuclear cells. The technician amplifies the DNA by PCR using pairs of primers specific for the FcγRIIA and FcγRIIIA loci, then digests the amplified DNA using restriction enzymes (Koene, et al, (1997) “Fc-gamma-RIIIA-158 V/F polymorphism influences the binding of IgG by natural killer cell Fc-gamma-RIIIA, independently of the Fc-gamma-RIIIA-48 L/R/H phenotype” Blood 90:1109-1114; Jiang-, et al. (1996) “Rapid detection of the Fc-gamma-RIIA-H/R¹³¹ ligand-binding polymorphism using an allele-specific restriction enzyme digestion (ASRED)” J. Immunol. Methods 199:55-59. The results indicate that the patient is homozygous H at residue 131 of FcγRIIA and homozygous V at residue 158 of FcγRIIIA. From this result, the physician uses the reference index relating genotype group to category of treatment response to antibody maintenance therapy and predicts that the patient will have an excellent response to antibody maintenance therapy. The patient is prescribed a 4-week course of therapy (375 mg/m² once per week), to be administered at 6-month intervals. 3 years later, the patient is still in remission, having experienced no additional symptoms since the completion of induction therapy.

### Example 2

### B-CLL Maintenance

A patient with B-cell chronic lymphocytic leukemia (B-CLL) is found to be not responsive to alemtuzumab, an anti-CD52 antibody. Antibody dependent cell-mediated cytotoxicity is a major mechanism by which the antibody exerts therapeutic efficacy. H is physician arranges for a technician to prepare genomic DNA from a sample of peripheral blood mononuclear cells, amplifies the DNA by PCR using pairs of primers specific for the FcγRIIA and FcγRIIIA loci, and sends the samples to the lab to be sequenced. The results of the sequence analysis indicate that the patient's genotype is R/R¹³¹ FcγRIIA, F/F¹⁵⁸ FcγRIIIA. Because this genotype is predictive of poor responsiveness to antibody therapy, the health care payer decides not to approve the prescription of alemtuzumab maintenance therapy and instead approves the prescription of an alternate therapy (e.g., small molecule plus chemocombination), and maintains recurring appointments with the patient to carefully monitor the disease status of the patient.

### Example 3

### SLE Maintenance Therapy

A patient diagnosed with systemic lupus erythematosus is treated with a course of anti-CD20 therapy. Before administering the antibody, a blood sample from the patient is withdrawn to assess the number of peripheral B cells by minimal residual disease (MRD) flow cytometry. See Dass, et al. (2008) Arth. Rheum. 58:2993-2999; Vital, et al (2011) Arth. Rheum. 63:603-608; and Moreton, et al. (2005) J. Clin. Oncol. 23:2971-2979. Two months after therapy, her physician draws a blood sample from the patient and assesses the number of peripheral B cells using MRD flow cytometry, e.g., using a CD19-specific antibody to detect the target B cells, and a CD45-specific antibody to detect all leukocytes. The flow cytometry results indicate less than 3 CD19+ cell per microliter, i.e., complete depletion with the anti-CD20 antibody therapy, indicating high ADCC function. Based on the MRD-FC results alone, the pharmacy benefit management company approves the as needed regimen of anti-CD20.

### Example 4

### Follicular Lymphoma Maintenance Therapy

A patient diagnosed with asymptomatic, non-bulky, stage 3, follicular lymphoma has the genotype: R/R¹³¹ FcγRIIA, F/F¹⁵⁸ FcγRIIIA. Because this genotype is predictive of only poor responsiveness to rituximab antibody therapy, the healthcare payer does not approve rituximab therapy in induction therapy setting, and further decides not to administer rituximab maintenance therapy. Instead the physician is advised to opt for “watch and wait” strategy.

### Example 5

### Cell Depletion Assay

Highly sensitive MRD flow cytometry methods are described, e.g., in Rawstron et al. (2006) Leukemia 20:2102-2110; Dass, et al. (2008) Arth. Rheum. 58:2993-999; Vital, et al. (2011) Arth. Rheu. 63:603-608).

The Fcg receptor genotypes of participants may be determined using highly sensitive methods, e.g., a combination of FACS and/or nucleic acid or protein sequence. MRD flow cytometry is performed through the treatment protocol, e.g., immediately before therapy, on day 15 before the second infusion of rituximab, 1 month after the second infusion, and at 2-month intervals thereafter. Maximum sensitivity will be achieved by incorporating the following features: (1) analysis of 500,000 events in each test; (2) enumeration of each B-cell subset over 2 separate tests, using the core markers phycoerythrin (PE)-Cy5.5-conjugated CD19 (Caltag, South San Francisco, Calif.), PE-Cy7-conjugated CD38, and allophycocyanin (APC)-labeled CD27 (BD Biosciences, Oxford, UK) and light scatter characteristics to identify the primary B-cell subsets, as follows: naive=CD19+CD27−CD38+/−, memory=CD19+CD27+CD38+/−, and plasma cell precursors=CD19+(weak)CD27++CD38++; (3) incorporation and use of the additional markers fluorescein isothiocyanate (FITC)-labeled CD3 or CD24, PE-labeled CD86 or CD14, and APC-Cy7-conjugated CD45 or CD20 (all from BD Biosciences) to confirm the phenotype and exclude contamination (e.g., only CD19+(weak)CD27++CD38++events that were also CD86+CD24− were classified as plasma cell precursors in the first test, while only CD19+(weak)CD27++CD38++ events that were also CD3−CD14−CD20− were classified as plasma cell precursors in the second test); (4) in the gating strategy of each test, inclusion of a marker that would not be expressed by the population of interest, in order to minimize contamination by events with nonspecific binding; (5) design and use of an extensive sequential gating strategy; this template was used for all patients, maximizing the ability of operators to recognize small variations in protein expression profile, thereby reducing any artifacts; (6) requirement for <25% coefficient of variation on the absolute total B cell levels and naive, memory, and plasma cell precursor levels between the 2 tests; and (7) in each case, analysis of the results by two separate operators at the same facility. The limit of detection of the assay is typically defined as 0.002%, i.e., at least 10 events that satisfied all criteria in both tests, or an absolute count of 0.0001×10⁹/liter (100 per ml), for a total white cell count of 5×10⁹/liter.

An analysis of B-cell depletion, e.g., with regard to European League Against Rheumatism (EULAR) outcomes (Van Gestel, et al. (1996) Arth. Rheum. 39:34-40), is performed, e.g., using chi-square tests, and the analysis of continuous variables done using Mann-Whitney tests or the like. Thus a study is performed to show that the outcome from antibody maintenance therapy, e.g., rituximab, in RA can be correlated with the amount of B-cell depletion which might be determined prior to any treatment. The quantitation may be useful in determining when and how to treat an individual.

FcγRIIIA V/F¹⁵⁸ and FcγRIIA H/R¹³¹ polymorphisms may also be determined for all participants, and each participant placed in one of the nine genotype groups. The outcomes, stratified according to the genotypes, are also evaluated as described above. The study indicates that the combination of FcγR polymorphisms, and the B-cell numbers before and after treatment provide valuable predictions on antibody maintenance therapy outcome.

### Example 6

### Cell Proliferation or Repopulation Assay

NK cells may be evaluated using standard procedures, and using commercially available reagents. Human NK cells may be isolated, e.g., using EasySep® Human NK Cell Enrichment Kit (Stemcell Technologies); or Bryceson, et al. (2010) Methods In Molecular Biology 612:335-352. Normalization methods may be used to determine absolute number or relative numbers of NK cells. Similar reagents may be used to detect cells using FACS equipment and methodologies.

A study is performed to show that the outcome from antibody maintenance therapy, e.g., rituximab, in an oncology setting can be correlated with the population of NK cells or measure of NK cell function, which might be determined prior to any treatment. Various studies in use of rituximab include Hochster, et al. (2009) J. Clin. Oncol. 27:1607-14 (advanced indolent lymphoma); Hainsworth, et al. (2005) J. Clin. Oncol. 23:1088-95 (indolent B-NHL); and Weng and Levy (2003) J. Clin. Oncol. 21:3940-47 (follicular lymphoma). Antibody maintenance therapy, e.g., with rituximab, can be performed, and participants monitored for NK cell numbers to show that the responsiveness, which is mediated in part by these cell types, correlates with population and population recovery. Additional assays involve the inclusion of IL-2 to ascertain whether the NK-cells in the patients can be activated. The quantitation may be useful in determining when and how to treat an individual. The initial NK cell population parameters will be useful in determining both initial induction therapy and subsequent maintenance therapy regimen.

### Example 7

### Follicular Lymphoma Study with Rituximab Maintenance Therapy

A study design can be used much like “LBA-6 Results of Eastern Cooperative Oncology Group Protocol E4402 (RESORT): A Randomized Phase III Study Comparing Two Different Rituximab Dosing Strategies for Low Tumor Burden Follicular Lymphoma” reported in part in poster by Kahl, et al. (2011) American Society of Hematology 2011, San Diego, Calif. on Tuesday, Dec. 13, 2011, 7:30 AM-9:00 AM; Hall AB (San Diego Convention Center). The study was directed to evaluate response of patients to antibody therapy. Similar evaluation methods may be utilized for evaluating response to antibody maintenance treatment, e.g., evaluation criteria have already been developed in studies on treatment of follicular lymphoma.

The data for determining relationship of responsiveness to antibody maintenance therapy to FcγRIIIA and FcγRIIA genotypes will be determined by retrospective determination of the genotypes after responsiveness measures have already been collected. The availability of samples for genotype determination is especially useful since the response outcomes over many years are already collected. FcγRIIIA V/F¹⁵⁸ and FcγRIIA H/R¹³¹ polymorphisms are determined using highly sensitive methods, e.g., a combination of FACS and/or nucleic acid or protein sequence, for all patients, and the patients are each placed in one of the nine patient genotype groups.

The Kahl follicular lymphoma study included sample archiving which now allows for retrospective evaluation of genotypes of patients. Samples are evaluated for FcγR polymorphisms to determine whether patient responsiveness to the applied therapy stratifies according to patient genotype. In particular, the placement of patients into genotype groups I-IX of the 3×3 matrix of FcγR IIIA and HA is tested for correlations with various subsets of patient outcomes. Among the subsets are (1) whether the exclusion criteria (e.g., unsuitability for inclusion in the antibody maintenance therapy phase) correlate with one or more groups of the matrix, (2) whether responsiveness to the treatments tested correlate with one or more of the groups of the matrix, e.g., whether the various responder subsets are enriched or underrepresented in genotype groups I, II, III, IV, and/or VII (or selected combinations thereof, e.g., I and II, or I and IV, or I and II and III, or I and IV and VII, or I and II and III and IV, or I and II and IV and VII) of the 3×3 matrix, (3) whether the non-responder subsets are enriched or underrepresented in groups IX, VIII, VI, and/or V (or combinations thereof, e.g., IX and VIII, or IX and VI, or IX and VI and VIII) of the 3×3 matrix, and/or (4) whether certain side effects are enriched or underrepresented in certain groups of the 3×3 matrix. Appropriate statistical analysis is performed to determine the statistical variation in the tested hypotheses.

Among specific issues to be evaluated include: (1) whether the patients not eligible for randomization to MR (maintenance rituximab) or RR (rituximab retreatment) correspond selectively to genotype groups [IX]; [VI+IX]; [VIII+IX]; [VI+VIII+IX]; or [V+VI+VIII+IX] or other groupings, which relate to lower ADCC functional subsets; (2) whether better (e.g., ‘complete’) responders to induction therapy correspond selectively to particular genotype groups, e.g., genotype groups [I]; [I+II]; [I+IV]; [I+II+IV]; [I+II+III]; [I+IV+VII]; [I+II+III+IV]; [I+II+IV+VII]; [I+II+III+IV+VII] or other groupings, which relate to higher ADCC functional subsets; (3) whether there is selection of better, (e.g., “complete”) responders to antibody maintenance therapy (MR in the Kahl study) or antibody retreatment (RR in the study) therapy in particular genotype groups, e.g., genotype groups [I]; [I+II]; [I+IV]; [I+II+IV]; [I+II+III]; [I+IV+VII]; [I+II+III+IV]; [I+II+IV+VII]; [I+II+III+IV+VII] or other groupings where higher ADCC functions would be expected; (4) whether treatment failure patients to antibody maintenance therapy (MR in this study) or antibody retreatment (RR) therapy correspond selectively to genotype groups [IX]; [VI+IX]; [VIII+IX]; [VI+VIII+IX]; or [V+VI+VIII+IX] or other groupings, which relate to lower ADCC functional subsets. In addition, upon review of treatment failure (the Time To Treatment Failure) in the antibody maintenance (MR) and antibody retreatment (RR) therapies, (5) can the time to treatment failure (TTTF) be significantly longer when patients are stratified into particular genotype groups, e.g., [I]; [I+II]; [I+IV]; [I+II+IV]; [I+II+III]; [I+IV+VII]; [I+II+III+IV]; [I+II+IV+VII]; or [I+II+III+IV+VII] (subsets exhibiting higher ADCC functions) as compared to genotype groups [IX]; [VI+IX]; [VIII+IX]; [VI+VIII+IX]; or [V+VI+VIII+IX] or other groupings, which exhibit lower ADCC functional subsets; and (6) is there change in responsiveness in genotype group V compared to either I or IX. Similarly, in review of another measure of treatment failure (the Time To first Cytotoxic Treatment) in the antibody maintenance (MR) and antibody retreatment (RR) arms, (7) is there selection among the “not failed” patients, e.g., remaining free of cytotoxic therapy at 3, 4, 5, and/or 6 years, for particular genotype groups or subsets, e.g., [I]; [I+II]; [I+IV]; [I+II+IV]; [I+II+III]; [I+IV+VII]; [I+II+III+IV]; [I+II+IV+VII]; or [I+II+III+IV+VII]; or (8) is there selection among the “failed” patients not remaining free of cytotoxic therapy at 3, 4, 5, and/or 6 years for particular genotype groups, e.g., [IX]; [VI+IX]; [VIII+IX]; [VI+VIII+IX]; or [V+VI+VIII+IX], which exhibit lower ADCC functions; or (9) in the antibody maintenance (MR) and antibody retreatment (RR) arms, preferably at 5 or 6 years, is it possible to identify genotype group subsets of patients remaining substantially free of cytotoxic therapy, e.g., [I]; [I+II]; [I+IV]; [I+II+IV]; [I+II+III]; [I+IV+VII]; [I+II+III+IV]; [I+II+IV+VII]; [I+II+III+IV+VII] as contrasted with [IX]; [VI+IX]; [VIII+IX]; [VI+VIII+IX]; or [V+VI+VIII+IX].

The patient data should also be queried to determine whether (10) the genotype grouping subsets exhibit significant difference between antibody maintenance (MR) and antibody retreatment (RR) therapy; and (11) when antibody maintenance (MR) therapy is different, preferably better, than antibody retreatment (RR) therapy, the dosing regimens and doses which provide the best differences identified. The patient data should also be evaluated to determine (12) whether the failed patients, whether induction plus antibody maintenance (MR) or plus antibody retreatment (RR) therapy, are selectively in particular genotype groups, e.g., [IX]; [VI+IX]; [VIII+IX]; [VI+VIII+IX]; or [V+VI+VIII+IX].

Thus, retrospective outcome data may be combined with genotype data to evaluate the correlations. In a prospective study, samples for individuals may also be collected and evaluated to determine whether an ADCC functional assay can be useful to provide useful prognosis of treatment response.

Further analyses may include whether PFS (Progression Free Survival) or QOL (Quality of Life) information or evaluation scores at various time points (e.g., 3, 4, 5, or 6 years) can be determined, and related to genotype groups. Pooling the MR and RR data sets may make sense if there are not significant differences between the groups, which may allow for better statistical power in the existing study.

### Example 8

### Rituximab Maintenance Therapy in SLE

An analogous study to the above may be performed using rituximab antibody maintenance therapy in SLE. See, e.g., Merrill, et al. (2010) Arthritis Rheum. 62:222-33.

FcγR IIIA V/F¹⁵⁸ and FcγR HA H/R¹³¹ polymorphisms are determined using highly sensitive methods, e.g., a combination of FACS and/or nucleic acid or protein sequence, for all participants, and each participant is placed in one of the nine patient genotype groups. Genotypes of excluded patients would preferably also be collected.

As described, a number of scoring systems exist to evaluate lupus, e.g., the SLEDAI, SELENA-SLEDAI, and BILAG indices, and the ACR guidelines. The method of antibody maintenance therapy would be designed to be more frequent as compared to the dosing in Merrill, e.g., repeated courses at more frequent intervals, with intervals selected to maintain the serum concentration of the antibody to a more constant tolerated level. As most antibodies, e.g., rituximab, have a 2-3 week serum half-life, the courses of administration may be selected to be closer to, e.g., 4-5 half-lives or 10-15 weeks. The range of concentration between beginning and end after 4 half-lives is 16-fold, and for 5 half-lives is about 32-fold, so dosing before reaching those time points correspond to less than about 20 or 40 fold, respectively. As described above, the 3A and 2A genotypes may be determined to detect the relation of response with genotype.

Institutional review board approval is obtained, preferably conducted in accordance with FDA Good Clinical Practice guidelines and the Health Insurance Portability and Accountability Act of 1996. Patients provide written informed consent prior to participation. Patients: Inclusion criteria are developed, e.g., age 16-75 years; a history of meeting 4 American College of Rheumatology (ACR) criteria for SLE (as defined in American College of Rheumatology Ad Hoc Committee on systemic lupus erythematosus response criteria (2004) “The American College of Rheumatology response criteria for systemic lupus erythematosus clinical trials: measures of overall disease activity” Arth. Rheum. 50:3418-26), including a positive test for antinuclear antibodies; active disease at screening, defined as ≧1 organ system with a British Isles Lupus Assessment (BILAG) A score (severe disease activity) or ≧2 organ systems with a BILAG B score (moderate disease activity) (see Hay, et al. (1993) Q. J. Med. 86:447-58; Isenberg and Gordon (2000) Lupus 9:651-4); and stable use of 1 immunosuppressive drug at entry, which might be continued during the trial.

Patients are excluded for severe central nervous system or organ-threatening lupus or any other active conditions requiring significant use of steroids or recent treatment with a cyclophosphamide or a calcineurin inhibitor (within 12 weeks of screening); a history of cancer or serious recurrent or chronic infection; uncontrolled medical disease; pregnancy or planning pregnancy; previous treatment with B cell-targeted therapy; aspartate aminotransferase or alanine aminotransferase level>2.5-fold the upper limit of normal (ULN); amylase or lipase level>2-fold the ULN; neutrophil count<1.0×103/μl; positive results of hepatitis B or hepatitis C serology; hemoglobin concentration<7 gm/dl (unless caused by hemolytic anemia due to SLE); platelet count<10,000/μl; and serum creatinine level>2.5 mg/dl.

Study design: An EXPLORER type protocol performs a trial which is a randomized, double-blind, placebo-controlled, multicenter (55 centers) North American study evaluating the efficacy and safety of rituximab compared with placebo in patients with SLE who were receiving background immunosuppressants and prednisone.

Patients may be randomized at a 2:1 ratio to receive intravenous rituximab (e.g., four 1,000-mg per intravenous infusion 2 months apart maintenance therapy) or placebo, which is added to prednisone and to a baseline immunosuppressive regimen. Each infusion might be accompanied by intravenous administration of acetaminophen, diphenhydramine, and methylprednisolone (100 mg). The BILAG index may be used to assess SLE activity. To ensure inclusion of only patients with significantly active disease, minimum disease severity at entry is stringently defined, e.g., as ≧1 domain with a BILAG A score or ≧2 domains with a BILAG B score, despite background treatment with either azathioprine (AZA; 100-250 mg/day), mycophenolate mofetil (MMF; 1-4 gm/day), or methotrexate (MTX; 7.5-27.5 mg/week). After screening, eligible patients continue their immunosuppressant therapy and received additional daily oral prednisone (e.g., 0.5 mg/kg, 0.75 mg/kg, or 1.0 mg/kg), based on the BILAG score at entry and the amount of steroids already being taken at the time of entry. Steroids may be tapered beginning on day 16, with the goal of reaching a dosage of ≦10 mg/day over 10 weeks and ≦5 mg/day by week 36.

Clinical assessments: Patients are evaluated regularly, e.g., monthly with the BILAG index and the Lupus Quality of Life (LupusQol) index (based on the Short Form 36 [SF-36] with additional components including pain and fatigue) (Yu, et al. (2006) Ann. Rheum. Dis. 65 Suppl. 11:601; and Ware and Sherbourne (1992) Med. Care 30:473-83). The BILAG index may be used to assess response, and scores might be converted to numeric values (A=9, B=3, C=1, D=0, E=0) (Ehrenstein, et al. (1995) Br. J. Rheum. 34:257-60) to enable evaluation of fluctuating global summary scores.

Primary end points: The effect of placebo versus rituximab in achieving and maintaining a major clinical response, a partial clinical response, or no clinical response at study evaluation points, e.g., week 24, are assessed, e.g., using each of the 8 BILAG index organ system scores.

A major clinical response might be defined as achieving BILAG C scores or better in all organs at a selected time point, e.g., week 24, without experiencing a severe flare (1 new domain with a BILAG A score or 2 new domains with a BILAG B score), e.g., from day 1 to week 24, and maintaining this response without a moderate or severe flare (≧1 new domains with a BILAG A or B score) to a select point, e.g., week 52. A partial clinical response might be defined as (1) achieving BILAG C scores or better at a select time, e.g., week 24, and maintaining this response without a new BILAG A or B score for 16 consecutive weeks, (2) achieving no more than 1 organ with a BILAG B score at a select time, e.g., week 24, without achieving ≧1 new BILAG A or B score to, e.g., week 52, or (3) achieving a maximum of 2 BILAG B scores at week 24 without developing BILAG A or B scores in new domains until week 52 if the baseline BILAG score for the patient is 1 A score plus ≧2 B scores, ≧2 A scores, or ≧4 B scores. No clinical response might be defined as failure to meet the definition of a major clinical response or a partial clinical response. Patients who terminated the study early might be scored as having no clinical response, but the genotype of such patients should also be determined.

Secondary end points: Secondary end points might include: (1) the time-adjusted area under the curve minus baseline (AUCMB) of the BILAG score over 36 and 52 weeks, (2) the proportion of patients who achieved a major clinical response (excluding a partial clinical response) and the proportion of patients with a partial clinical response (including a major clinical response) at week 52, (3) the proportion of patients with a BILAG C score or better in all organs at week 24, (4) the time to the first moderate or severe disease flare, (5) improvement in quality of life as measured by the LupusQoL, and (6) the proportion of patients who achieved a major clinical response with a prednisone dosage of <10 mg/day from week 24 to week 52.

Subgroup analyses of the primary and secondary end points could be preplanned for the following factors: race (African American/Hispanic versus others), age (≦40 years or >40 years), sex, assigned prednisone dose, background immunosuppressant, duration of lupus, long-term prednisone therapy, baseline BILAG A score, baseline BILAG-defined mucocutaneous or musculoskeletal system involvement, and baseline biomarkers.

FcγRIIIA V/F¹⁵⁸ and FcγRIIA H/R¹³¹ polymorphisms are determined using highly sensitive methods, e.g., a combination of FACS and/or nucleic acid or protein sequence, for all participants, and each participant is placed in one of the nine patient genotype groups. Genotypes of excluded patients would preferably also be collected. Depletion and repopulation patterns of the B cell subsets (naïve, memory, and plasma precursors) are measured by MRD-flow cytometry at various time points:pre-treatment just before dosing; and then every month for 12 months.

Safety assessments: The incidence and severity of adverse events (AEs) would likely be classified using the National Cancer Institute Common Toxicity Criteria for Adverse Events (version 3.0). Serious adverse events (SAEs), infusion-related AEs (an AE occurring during or within 24 hours following completion of the infusion of a study drug), and infection-related AEs would be summarized independently. Serum chemistries, hematologic parameters, urinalysis results, quantitative IgG levels, T cell and B cell counts, human anti-chimeric antibody (HACA) levels, complement levels, and autoantibody levels could be monitored.

Statistical analysis: The proportion of patients achieving a major clinical response, a partial clinical response, or no clinical response would be compared between the placebo and rituximab groups using, e.g., the stratified Wilcoxon's rank sum test, with the initial prednisone dose and race/ethnicity as stratification factors. Results might be expressed as the proportion of patients in each of 6 cells (2 treatment groups×3 response categories), and the value computed to compare the graded response across treatment arms. Two-sided P values less than 0.05 would typically be considered significant. The analyses are often performed using SAS software, e.g., version 9.1 (SAS Institute, Cary, N.C.).

The data are evaluated to determine if the treatment responses of individuals can be related to the genotype groups, as described above. In particular, whether the response rates of the high ADCC function genotype groups (e.g., genotype groups I, II, III, IV, and VII, or subsets among them) are distinguishable from response rates of low ADCC function genotypes (e.g., genotype groups IX, VIII, VI, and V, or subsets among them). Where statistically significant differences are observed between the genotypes, stratification of patients according to genotype may provide useful predictions for responsiveness to the antibody maintenance therapy. In particular, certain genotype groups may be converted with sustained maintenance therapy into relatively higher response outcomes. Additionally, patients of the high response genotypes may be selectively included into clinical trial treatment groups so as to provide a higher responsiveness outcome compared to unstratified target population groups.

### Example 9

### Rituximab Maintenance Therapy in RA

A trial is performed in an autoimmune indication using an antibody retreatment regimen, e.g., rituximab. FcγRIIIA V/F¹⁵⁸ and Fcγ R IIA H/R¹³¹ polymorphisms are determined using highly sensitive methods, e.g., a combination of FACS and/or nucleic acid or protein sequence, for all participants, and each participant is placed in one of the nine patient genotype groups. Genotypes of the excluded patients are also collected. In prospectively designed trials, depletion and repopulation patterns of the B cell subsets (naïve, memory, and plasma precursors) are measured by MRD-flow cytometry at various time points:pre-treatment just before dosing; and then every month for 12 months.

Analogous to the study described by Kahl in Example 7, a trial can be performed or designed using treatment response measures appropriate in RA. Examples of clinical studies in rheumatoid arthritis include Mease, et al. (2010) J. Rheumatol. 37:917-927 (SUNRISE); Keystone, et al. (2007) Arthritis Rheum, 56:3896-908; Emery, et al. (2010) Ann. Rheum. Dis. 69:1629-635 (SERENE); and Rubbert-Roth, et al. (2010) Rheumatology (Oxford) 49:1683-1693 (MIRROR). ACR70, ACR50, DAS remission, or DAS-low disease are measured and correlated to Fcγ receptor polymorphisms and the ADCC function. Other objective scoring systems exist in other autoimmune conditions, e.g., for lupus, multiple sclerosis, etc.

Data analysis of the genotype stratified response of patients are evaluated as described above.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1-55. (canceled)

56. A method selected from:
A) a method for predicting responsiveness of a subject having an antibody dependent cell-mediated cytotoxicity (ADCC)-treatable disease or disorder to an antibody maintenance therapy, the method comprising:
(a) determining a genotype of the subject for one or more Fcγ receptor polymorphisms affecting ADCC activity, wherein the Fcγ receptor polymorphism is selected from a FcγRIIA polymorphism and a FcγRIIIA polymorphism; and
(b) predicting a responsiveness of the subject to an antibody maintenance therapy based upon the determined genotype of the Fcγ receptor polymorphism;
B) a method for selecting a subject having an antibody dependent cell-mediated cytotoxicity (ADCC)-treatable disease for treatment with an antibody maintenance therapy, comprising:
(a) determining a genotype of the subject for one or more Fcγ receptor polymorphisms affecting ADCC activity, wherein the Fcγ receptor polymorphism is selected from a FcγRIIA polymorphism and a FcγRIIIA polymorphism; and
(b) selecting or excluding the subject for treatment with the antibody maintenance therapy based on the determined genotype of the Fcγ receptor polymorphism: or
C) a method of treating a subject having an antibody dependent cell-mediated cytotoxicity (ADCC)-treatable disease or disorder with an antibody maintenance therapy, comprising:
(a) determining a genotype of the subject for one or more Fcγ receptor polymorphisms affecting ADCC activity, wherein the Fcγ receptor polymorphism is selected from a FcγRIIA polymorphism and a FcγRIIIA polymorphism;
(b) stratifying the subject into a responsiveness group based on the determined genotype, and selecting or excluding the subject for antibody maintenance therapy based on the stratification; and
(c) administering to the selected subject the antibody maintenance therapy regimen.

57. The method according to claim 56 embodiment C, wherein:
(a) the stratifying into a responsiveness group is carried out by comparing the determined genotype of the subject to a reference stratification that relates responsiveness to antibody maintenance therapy for the ADCC treatable disease to genotypes of the Fcγ receptor polymorphism;
(b) the antibody in the maintenance therapy is administered regularly or as-needed; or
(c) the responsiveness group corresponds to a combined genotype group corresponding to genotype groups I, I+II, I+IV, I+II+IV, I+II+III+IV+VII, IX, VIII+IX, VI+IX, or VI+VIII+IX.

58. The method according to claim 56, wherein:
A) the Fcγ receptor polymorphism comprises:
(a) the FcγRIIA polymorphism or the FcγRIIIA polymorphism;
(b) the FcγRIIA polymorphism and the FcγRIIIA polymorphism;
(c) an FcγRIIA polymorphism at amino acid residue 131;
(d) an FcγRIIA polymorphism at amino acid residue 131 which comprises genotypes H/H¹³¹, H/R¹³¹ or R/R¹³¹, wherein H is histidine and R is arginine;
(e) an FcγRIIIA polymorphism at amino acid residue 158; or
(f) an FcγRIIIA polymorphism at amino acid residue 158 which comprises genotypes V/V¹⁵⁸, V/F¹⁵⁸ or F/F¹⁵⁸, wherein V is valine and F is phenylalanine;
B) the predicting, stratifying, or treating is based on FcγRIIA polymorphism at amino acid residue 131 and FcγRIIIA polymorphism at amino acid residue 158, and comprises genotype groups:
H/H¹³¹ for FcγRIIA and V/V¹⁵⁸ for FcγRIIIA;
H/H¹³¹ for FcγRIIA and V/F¹⁵⁸ for FcγRIIIA;
H/H¹³¹ for FcγRIIA and F/F¹⁵⁸ for FcγRIIIA;
H/R¹³¹ for FcγRIIA and V/V¹⁵⁸ for FcγRIIIA;
R/R¹³¹ for FcγRIIA and V/V¹⁵⁸ for FcγRIIIA;
H/R¹³¹ for FcγRIIA and V/F¹⁵⁸ for FcγRIIIA;
H/R¹³¹ for FcγRIIA and F/F¹⁵⁸ for FcγRIIIA;
R/R¹³¹ for FcγRIIA and V/F¹⁵⁸ for FcγRIIIA; and
R/R¹³¹ for FcγRIIA and F/F¹⁵⁸ for FcγRIIIA;
C) the predicting, stratifying, or treating is based on FcγRIIA polymorphism at amino acid residue 131 and FcγRIIIA polymorphism at amino acid residue 158, and:
(a) H/H¹³¹ FcγRIIA and V/V¹⁵⁸ FcγRIIIA is predictive of excellent responsiveness to the antibody maintenance therapy;
(b) H/H¹³¹ FcγRIIA and V/F¹⁵⁸ FcγRIIIA; H/H¹³¹ FcγRIIA and F/F¹⁵⁸ FcγRIIIA; H/R¹³¹ FcγRIIA and V/V¹⁵⁸ FcγRIIIA; and R/R¹³¹ FcγRIIA and V/V¹⁵⁸ FcγRIIIA is predictive of good responsiveness to the antibody maintenance therapy;
(c) H/R¹³¹ FcγRIIA and V/F¹⁵⁸ FcγRIIIA is predictive of moderate responsiveness to the antibody maintenance therapy;
(d) H/R¹³¹ FcγRIIA and F/F¹⁵⁸ FcγRIIIA; and R/R¹³¹ FcγRIIA and V/F¹⁵⁸ FcγRIIIA is predictive of weak responsiveness to the antibody maintenance therapy; and
(e) R/R¹³¹ FcγRIIA and F/F¹⁵⁸ FcγRIIIA is predictive of poor responsiveness to the antibody maintenance therapy;
D) the genotype of the Fcγ receptor polymorphism is determined using:
(a) an antibody capable of detecting the polymorphism; or
(b) a nucleic acid of the subject, wherein the nucleic acid is genomic DNA or expressed RNA;
E) the subject:
(a) has had or will have induction therapy, which may comprise chemotherapy or antibody therapy; or
(b) previously received or is receiving antibody maintenance therapy for the disease or disorder;
F) the ADCC-treatable disease or disorder is selected from a neoplastic disease, an autoimmune disease, an inflammatory disorder, a microbial infection, or allograft rejection, including embodiments wherein:
(a) the antibody for the maintenance therapy of the neoplastic disease comprises an anti-CD19 antibody, anti-CD20 antibody, anti-CD22, anti-CD25 antibody, anti-CD30 antibody, anti-CD33 antibody, anti-CD52 antibody, anti-EGFR, anti-EphA2 antibody, anti-GD2 antibody, anti-G250 antibody, anti-ErB2 antibody, anti-folate beta receptor antibody, or anti-phosphatidylserine antibody;
(b) the neoplastic disease is B-cell non-Hodgkin's lymphoma (B-NHL) and the antibody is an anti-CD20 antibody, preferably wherein the antibody for maintenance therapy is selected from rituximab, ofatumumab, ibritumomab, tositumomab, veltuzumab, and obinutuzumab; or
(c) the ADCC-treatable disease or disorder is an autoimmune disease, including wherein: the autoimmune disease is selected from systemic lupus erythematosus, lupus nephritis, rheumatoid arthritis, Sjögren's syndrome, Grave's disease, and multiple sclerosis; and preferably wherein the antibody for the maintenance therapy of the autoimmune disease comprises an anti-CD19 antibody, or anti-CD20 antibody, or anti-CD22 antibody; and wherein the antibody for the maintenance therapy comprises an anti-CD20 antibody selected from rituximab, ofatumumab, ibritumomab, tositumomab, veltuzumab, and obinutuzumab;
G) the predicted responsiveness, the selecting, or stratification is reported in electronic, web-based, or paper form to the subject, a health care payer, third party payer, a health care provider, a physician, a pharmacy benefits manager, or a government office;
H) the method further comprises a decision:
(a) to treat the subject with the antibody maintenance therapy; or
(b) to not treat the subject with the antibody maintenance therapy; or
I) the method further comprises a step of treating the subject with the antibody maintenance therapy, which antibody maintenance therapy may include monotherapy with the antibody, or antibody maintenance therapy in combination therapy, e.g., with chemotherapy or radiation therapy.

59. The methods of claim 56, further comprising measuring the level of ADCC function of the subject.

60. The methods of claim 59, wherein:
(a) the ADCC function is measured using ADCC effector cells obtained from the subject, which ADCC effector cells may comprise macrophages, natural killer (NK) cells, neutrophils, eosinophils or combinations thereof;
(b) the ADCC function is measured using a target cell radiolabel release assay, target cell enzyme release assay, or target cell depletion/repopulation assay;
(c) the ADCC function is determined by measuring depletion or repopulation of a cell population targeted by an antibody induction therapy or an antibody maintenance therapy;
(d) the subject selected for antibody maintenance therapy has at least same levels, 10% or more, 20% or more, 30% or more, 40% or more, 50% or more, 60% or more, 70% or more 80% or more, or 90% or more of ADCC capacity/function as compared to a control subject, wherein the control subject is a healthy subject or a treatment naïve subject afflicted with the ADCC treatable disease or disorder;
(e) the predicted responsiveness, the selecting, or stratification is reported in electronic, web-based, or paper form to the subject, a health care payer, third party payer, a health care provider, a physician, a pharmacy benefits manager, or a government office; or
(f) the method further comprises making a decision which determines the duration, timing, frequency, dose, or combination therapies accompanying the antibody maintenance therapy.

61. A method selected from:
A) a method for selecting a treatment option for an ADCC treatable disease, comprising:
(a) determining a genotype of a subject for one or more Fcγ receptor polymorphisms affecting ADCC activity, wherein the human subject has an ADCC treatable disease; and
(b) selecting one or more treatment options from a panel of available treatment options based on the determined genotype of the Fcγ receptor polymorphism, wherein the treatment options comprise at least antibody maintenance therapy for the ADCC treatable disease;
B) a method of selecting a human subject for inclusion in clinical trials or conducting a clinical trial to evaluate an antibody maintenance therapy for treating an ADCC treatable disease, comprising:
(a) determining a genotype of a human subject having an ADCC treatable disease for an Fcγ receptor polymorphism affecting ADCC activity; and
(b) using the determined genotype of the Fcγ receptor polymorphism in deciding inclusion or exclusion of the subject in the clinical trial for the antibody maintenance therapy;
C) a method for predicting responsiveness of a human subject having an ADCC-treatable disease or disorder to an antibody maintenance therapy, the method comprising:
(a) measuring depletion or repopulation of a cell population targeted by an antibody induction therapy or an antibody maintenance therapy to obtain a result; and
(b) predicting the responsiveness of the human subject to an antibody maintenance therapy based upon the result; or
D) a method for treating a human subject having an ADCC-treatable disease or disorder with an antibody maintenance therapy, comprising:
(a) measuring depletion or repopulation of a cell population targeted by an antibody induction therapy or an antibody maintenance therapy to obtain a result;
(b) classifying the human subject into a responsiveness groups based on the result to obtain a classification; and
(c) administering antibody maintenance therapy to the human subject either as a regular regimen or an as-needed regimen based on the classification,
wherein the regimen is selected based on the human subject's classification in a responsiveness group.

62. The method according to claim 61 embodiment C or D, wherein:
(a) the ADCC-treatable disease or disorder is a neoplastic disease, an autoimmune disease, an inflammatory disorder, a microbial infection, or allograft rejection;
(b) the ADCC-treatable disease or disorder is a neoplastic disease which is B-cell non-Hodgkin's lymphoma (B-NHL);
(c) the ADCC-treatable disease or disorder is a neoplastic disease which is B-cell non-Hodgkin's lymphoma (B-NHL) which is follicular lymphoma (FL);
(d) the ADCC-treatable disease or disorder is an autoimmune disease which is rheumatoid arthritis;
(e) the targeted cell population is B cells;
(f) the targeted cell population is B cells and the antibody induction therapy is induction therapy comprising an anti-CD20 antibody;
(g) the targeted cell population is B cells and the antibody induction therapy is induction therapy comprising rituximab; or
(h) the method further comprises genotyping the human subject for an FcγRIIA polymorphism and an FcγRIIIA polymorphism, wherein predicting responsiveness of the human subject to an antibody maintenance therapy is based upon the result of the depletion measurement and the result of the genotyping.

63. The method according to claim 61 embodiment D, wherein the antibody maintenance therapy is administered regularly or as-needed.

64. A kit for predicting responsiveness of a human subject having an ADCC-treatable disease or disorder to an antibody maintenance therapy, the kit comprising one or more of components:
(a) an element for genotyping the human subject to identify a FcγRIIA polymorphism;
(b) an element for genotyping the human subject to identify a FcγRIIIA polymorphism;
(c) a reference stratification that correlates a genotype in the human subject with a human subject group having known treatment responsiveness to the antibody maintenance therapy;
(d) an element for measuring the number of cells in a sample from an human subject having an ADCC-treatable disease or disorder, wherein the cells are cells targeted for depletion by an antibody induction therapy or an antibody maintenance therapy; and
(e) a reference stratification that correlates the extent of depletion of targeted cells following antibody induction therapy or antibody maintenance therapy with responsiveness to an antibody maintenance therapy.

65. The kit according to claim 64, wherein:
(a) the kit comprises two, three, or four of the components;
(b) at least one of the reference stratifications is provided in electronic, web-based, or paper form;
(c) the FcγRIIA polymorphism is a polymorphism at residue 131;
(d) the FcγRIIIA polymorphism is a polymorphism at residue 158;
(e) the ADCC-treatable disease or disorder is a neoplastic disease, an autoimmune disease, an inflammatory disorder, a microbial infection, or allograft rejection;
(f) one of the elements is an antibody reagent; or
(g) the element for measuring is specific for B cells.

66. The method of claim 56, embodiment A.

67. The method of claim 56, embodiment B.

68. The method of claim 56, embodiment C.

69. The method of claim 61, embodiment A.

70. The method of claim 61, embodiment B.

71. The method of claim 61, embodiment C.

72. The method of claim 61, embodiment D.
